(19) United States
(12) Patent Application Publication
(10) Pub. No.: US 2004/0063643 A1
(43) Pub. Date: Apr. 1, 2004

(54) Bombesin antagonists

(75) Inventors: Michael Higginbottom, Sandwich (GB); Suzanne Ross Kesten, Ann Arbor, MI (US); Russell Andrew Lewthwaite, Sandwich (GB); Martyn Clive Pritchard, Sandwich (GB); David James Rawson, Sandwich (GB); Robert Michael Schelkun, Ann Arbor, MI (US); Po-Wai Yuen, Ann Arbor, MI (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/425,758

(22) Filed: Apr. 28, 2003

Related U.S. Application Data
(60) Provisional application No. 60/398,132, filed on Jul. 23, 2002.

(30) Foreign Application Priority Data
May 3, 2002 (GB) 0210239.0

(51) Int. Cl. A61K 38/04; A61K 31/537; A61K 31/44; A61K 31/40

(52) U.S. Cl. 514/19; 514/237.8; 514/357; 514/408; 544/159; 546/336; 548/561

(57) ABSTRACT

Compounds of formula (I):

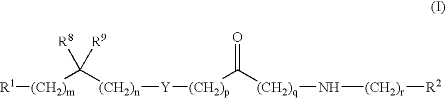

and their salts, solvates, prodrugs, etc., wherein the substituents have the values mentioned herein, are bombesin antagonists, which have utility in a variety of therapeutic areas including male and female sexual dysfunction, particularly female sexual dysfunction (FSD) especially wherein the FSD is female sexual arousal disorder (FSAD) and male erectile dysfunction (MED)

This invention relates to a class of bombesin antagonists, uses thereof, processes for the preparation thereof, intermediates used in the preparation thereof and compositions containing said inhibitors. These inhibitors have utility in a variety of therapeutic areas including male and female sexual dysfunction, particularly female sexual dysfunction (FSD) especially wherein the FSD is female sexual arousal disorder (FSAD) and male erectile dysfunction (MED)

According to a first aspect, the invention provides a class of compounds of formula (1).

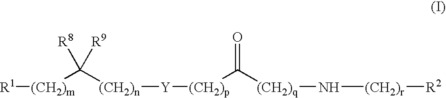

Wherein:

R¹ is selected from

a) aryl

b) aromatic heterocycle

c) CO₂R⁵

d) CONR⁵R⁶

e) NR⁵R⁷

f) OR⁵

g) C₁₋₆ alkyl

h) C₁₋₆cycloalkyl and

i) —C(O)—N-morpholine

Wherein group (a) may be optionally substituted by 1-3 groups each independently selected from NR⁵R⁵, N(R⁵)C(O)R⁵, NO₂, halo, OR⁵, R⁵ and R⁴NR⁵R⁵; and group (b) may be optionally substituted by 1-3 groups each independently selected from halo, R⁵ and OR⁵;

m is 0-2;

n is 0-2;

p is 0-2;

q is 0-2;

r is 0-4

Y is NR³ or CHR³;

R² is selected from

a) C₃₋₇ cycloalkyl,

b) aromatic heterocycle, optionally fused with a phenyl group,

c) aryl, wherein said aryl group may optionally be fused with a heterocycle or a C₃₋₇ cycloalkyl group, wherein said fused cycloalkyl moiety may also incorporate a C═O group,

d) Oaryl,

e) C₁₋₆ alkyl,

f) Adamantyl, and

g) C₁₋₆ alkenyl, optionally substituted by 1 or 2 phenyl,

wherein groups (a), (b), (c), (d) and (e) may be optionally substituted by 1-3 substituents selected from: R⁵, C₁₋₆ alkenyl, aryl, OR⁴, OR⁵, OH, CF₃, halo, SO₂R⁵, NO₂, SR⁵, CN, OCF₃, CO₂R⁵, C(O)R⁵, Oaryl, OR⁴aryl, R⁴OR⁵, C(NH)NR⁵R⁵, OC(O)C₁₋₁₆ alkyl and NR⁵R⁷;

R³ is selected from

a) C₁₋₆ alkyl,

b) C₁₋₆alkenyl,

c) C₁₋₆ alkynyl,

d) Aromatic heterocycle, optionally fused with phenyl,

e) Phenyl, optionally fused with phenyl, heterocycle or aromatic heterocycle,

said groups (a), (b), (c), (d) and (e) optionally substituted by 1-3 groups each independently selected from halo, CN, SR⁵, heterocycle, aromatic heterocycle, R⁵, OR⁷, C(O)NR⁵R⁷, SO₂NR⁵R⁷, NHSO₂R⁵, OH, CF₃, OR⁵, OR⁵OR⁵, NR⁵R⁷, CO₂H, CO₂R⁵, OC(O)R⁵, C₃₋₇ cycloalkyl group (wherein said cycloalkyl group may optionally be substituted by C₁₋₆ alkyl), CH₂OC(O)CH₃ and phenyl, wherein said phenyl may optionally be fused with a heterocycle, aromatic heterocycle, phenyl or C₃₋₇ cycloalkyl, said phenyl, fused phenyl, or aromatic heterocycle optionally substituted by 1-3 groups each independently selected from: phenyl, R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, HNC(O)R⁵, C₁₋₆ alkenyl, OCF₃, NO₂, halo, HNSO₂R⁵, SO₂NR⁵R⁵, C(O)NR⁵R⁵, C(NH)NR⁵R⁵, OR⁵, OC(O)R⁴-heterocycle, NR⁵R⁷, SR⁵ and tetrazole;

R⁴ is C₁₋₆ alkyl;

R⁵ is independently selected from H and C₁₋₆ alkyl, said alkyl groups optionally substituted by 1-3 groups each independently selected from halo or OH;

R⁶ is independently selected from H, heterocycle, OC₁₋₆ alkyl, C₁₋₆ alkyl, said alkyl groups optionally substituted by 1-3 groups each independently selected from halo or OH;

Or R⁵ and R⁶ can be taken together with the N atom to which they are attached to form a 5, 6 or 7-membered ring optionally containing a further hetero moiety selected from O, NH, or S;

R⁷ is selected from H and C₁₋₆ alkyl, said alkyl groups optionally substituted by an aryl group; and

R⁸ and R⁹ are both independently selected from H or C₁₋₆ alkyl; or R⁸ and R⁹ may combine to form a 3-7 membered cycloalkyl group. Optionally said cycloalkyl group may incorporate an atom or group selected from NR⁴, NH, O or S;

with the proviso that when R¹ is an aryl or aromatic heterocycle group, R² is a phenyl, pyridyl or pyrimidinyl group, said groups optionally substituted, R⁸ and R⁹ combine to form a 3-7 membered cycloalkyl group, Y is NR³ and m, p, q and r are 0; then R³ cannot be C₄₋₆ alkyl or C₁ alkyl substituted by phenyl, said phenyl group optionally substituted by 1-3 groups each independently selected from halo, OC₁₋₆ alkyl and NR⁵R⁷;

and the zwitterions, pharmaceutically acceptable salts, prodrugs, solvates and polymorphs thereof.

Aryl is defined as a 6-14 membered aromatic carbocycle. Aromatic heterocycle is defined as a 5 to 7 membered ring, containing from 1 to 4 heteroatoms, each independently selected from O, S and N, said ring optionally fused with aryl or heterocycle, said aromatic heterocycle optionally substituted by 1-3 groups each independently selected from: R⁵, OR⁵, NR⁵R⁵ and halo

Heterocycle is defined as a 3-8 membered ring containing from 1-3 heteroatoms, each independently selected from O, S and N, said ring being saturated or partially saturated, wherein optionally said ring may contain a C═O moiety, wherein said ring may be optionally substituted by 1-3 C₁₋₆ alkyl groups.

Halo includes fluoro, chloro, bromo and iodo groups.

Alkyl includes straight chain and branched chain. The term alkyl also includes those groups described by (CH₂)ₘ, (CH₂)ₙ, (CH₂)ₚ, (CH₂)_{q} and (CH₂)ᵣ.

A 6-14 membered aromatic carbocycle includes phenyl, naphthyl, indenyl, anthryl and phenanthryl.

The pharmaceutically acceptable salts of the compounds of the formula (I) include the acid addition and the base salts thereof.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

For a review on suitable salts see Berge et al, J. Pharm. Sci., 66, 1-19, 1977.

The pharmaceutically acceptable solvates of the compounds of the formula (I) include the hydrates thereof.

Also included within the present scope of the compounds of the formula (I) are polymorphs thereof.

Certain compounds of the formula (I) contain one or more asymmetric carbon atoms and therefore exists in two or more stereoisomeric forms. Where a compound of the formula (I) contains an alkenyl or alkenylene group, cis (E) and trans (Z) isomerism may also occur. The present invention includes the individual stereoisomers of the compounds of the formula (I) and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

A pharmaceutically acceptable salt of a compound of the formula (I) may be readily prepared by mixing together solutions of a compound of the formula (I) and the desired acid or base, as appropriate. The salt may precipitate from solution and be collected by filtration or may be recovered by evaporation of the solvent.

In a preferred embodiment of the invention R¹ is selected from:

a) aryl

b) aromatic heterocycle

c) CO₂R⁵

d) OR⁵,

wherein group a) may be optionally substituted by 1-3 groups each independently selected from NR⁵R⁵, N(R⁵)C(O)R⁵, NO₂, halo, OR⁵, R⁵ and R⁴NR⁵R⁵,

and wherein group b) may be optionally substituted by 1-3 groups each independently selected from halo, R⁵ and OR⁵.

More preferably, R¹ is selected from:

a) pyridyl

b) thienyl

c) phenyl

d) pyrrolyl

e) imidazolyl and

f) CO₂R⁵

wherein groups a), b), d) and e) may be optionally substituted by 1 or 2 groups each independently selected from methoxy, methyl and halo,

and wherein group c) may be optionally substituted by 1 or 2 groups each independently selected from methoxy, methyl, halo and nitro.

Yet more preferably, R¹ is selected from pyridyl, methoxy-pyridyl, thienyl, phenyl, difluorophenyl, methyl-pyrrolyl, CO₂Et, methyl-imidazolyl, nitrophenyl and methoxy-phenyl.

Most preferably, R¹ is selected from: 2-pyridyl, 5-methoxy-pyridin-2-yl, 2-thienyl, 3-thienyl, 2,6-difluorophenyl, N-methyl-pyrrol-2-yl, CO₂Et, 1-methyl-imidazol-4-yl, 2-nitro-phenyl, 2-methoxy-phenyl and 5-methoxy-pyridin-2-yl.

Preferably m is 0-1, more preferably 0.

Preferably n is 0-1, more preferably 1.

Preferably p is 0-1, more preferably 0.

Preferably q is 0-1, more preferably 0.

Preferably r is 0-1, more preferably 0.

Preferably Y is NR³.

Preferably R² is selected from:

a) C₃₋₇ cycloalkyl;

b) aromatic heterocycle, optionally fused with a phenyl group;

c) aryl, wherein said aryl group may optionally be fused with, a heterocycle or a C₃₋₇ cycloalkyl group, wherein said fused cycloalkyl moiety may also incorporate a C═O group;

d) OPh;

e) —CH₂OHCH₂Ph;

f) adamantyl; and

g) —CH═CHPh;

wherein groups (a), (b), (c) and (d) may be optionally substituted by 1-3 substituents selected from: C₁₋₆ alkyl, C₁₋₆ alkenyl, phenyl, OR⁴, OR⁵, OH, CF₃, halo, SO₂R⁵, NO₂, SR⁵, CN, OCF₃, CO₂R⁵, C(O)R⁵, Oaryl, OR⁴aryl, R⁴OR⁵, C(NH)NR⁵R⁵, OC(O)C₁₋₆ alkyl and NR⁵R⁷.

More preferably R² is a phenyl or naphthalene group, optionally substituted by 1-3 substituents selected from C₁₋₃ alkyl, CF₃, halo, OR⁵ and NR⁵R⁷. Yet more preferably R² is phenyl substituted by 2 substituents selected from C₁₋₃alkyl, halo and NR⁵R⁷.

Yet even more preferably R² is phenyl substituted by 2 substituents independently selected from Me, chloro, isopropyl and NMe₂. In a particularly preferred embodiment both phenyl substituents are the same. Most preferably, R² is 2,6-diisopropyl-phenyl.

Preferably R³ is selected from:

a) C₁₋₆alkyl;

b) C₁₋₆alkenyl;

c) C₁₋₆ alkynyl;

d) Aromatic heterocycle, optionally fused with phenyl; said aromatic heterocycle or fused heterocycle being optionally substituted by 1-3 substituents each independently selected from: halo, OC(O)CH₃ and —CH₂OC(O)CH₃; and

e) Phenyl, optionally fused with a heterocycle or aromatic heterocycle,

said phenyl or fused phenyl optionally substituted by 1-3 substituents each independently selected from: C₁₋₆ alkyl, C(O)NR⁵R⁷, SO₂NR⁵R⁷ and NHSO₂R⁵;

said groups (a), (b) and (c) optionally substituted by 1-3 groups each independently selected from halo, CN, SR⁵, heterocycle, aromatic heterocycle, OR⁷, OH, CF₃, OR⁵, OR⁵OR⁵, NR⁵R⁷, CO₂H, CO₂R⁵, C₃₋₇ cycloalkyl group (wherein said cycloalkyl group may optionally be substituted by C₁₋₆ alkyl) and phenyl, wherein said phenyl may optionally be fused with a heterocycle, phenyl or C₃₋₇ cycloalkyl, said phenyl, fused phenyl, or aromatic heterocycle optionally substituted by 1-3 groups each independently selected from: phenyl, R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, NHC(O)R⁵, C₁₋₆alkenyl, OCF₃, NO₂, halo, NHSO₂R⁵, SO₂NR⁵R⁵, C(O)NR⁵R⁵, C(NH)NR⁵R⁵, OR⁵, OC(O)R⁴-heterocycle, NR⁵R⁷, SR⁵ and tetrazole.

More preferably R³ is C₁₋₃ alkyl, optionally substituted by 1-2 groups each independently selected from OH, OR⁵, NR⁵R⁷, C₃₋₇ cycloalkyl, CO₂R⁵ and phenyl, wherein said phenyl may optionally be fused with a heterocycle, said phenyl or fused phenyl optionally substituted by 1-3 groups each independently selected from halo, NO₂, NHSO₂R⁵, SO₂NR⁵R⁵, C(O)NR⁵R⁵, C(NH)NR⁵R⁵, OR⁵ and NR⁵R⁷, or R³ is C₁₋₆ alkyl.

Yet more preferably R³ is C₁ alkyl, substituted by:

a) phenyl, optionally substituted by 1-3 groups each independently selected from: OH and C(O)NR⁵R⁵, or

b) C₃₋₇ cycloalkyl

or R³ is C₁₋₆ alkyl.

Most preferably R³ is 4-hydroxy-benzyl, cyclopropyl-methyl, isopropyl-methyl or

—CH₂Ph-(3-C(O)—NHEt).

Preferably R⁴ is methyl or —CH₂—.

Preferably R⁵ is H or C₁₋₆ alkyl optionally substituted by OH or trisubstituted by F

More preferably R⁵ is H or C₁₋₃ alkyl optionally substituted by OH or trisubstituted by F.

Preferably R⁶ is H or C₁₋₆ alkyl.

Preferably R⁷ is H or C₁₋₆ alkyl.

More preferably R⁷ is H or C₁₋₃ alkyl.

Preferably R⁸ and R⁹ combine to form a 3-7 membered cycloalkyl group, wherein said cycloalkyl group may optionally incorporate a heteroatom selected from NH, NR⁴, O or S.

More preferably R⁸ and R⁹ combine to form a 3-7 membered cycloalkyl group.

Most preferably R⁸ and R⁹ combine to form a cyclohexane group.

Preferably aryl is phenyl.

Aromatic heterocycle includes imidazole, pyrrole, thiophene and pyridine.

A preferred group of compounds is that in which each substituent R¹, m, R⁸, R⁹, n, Y, p, q, r and R² is as specified in the Examples given below.

In another embodiment of the invention, preferences for:

R¹; m; n; p; q; r; Y; R²; R³; R⁴; R⁵; R⁶; R⁷; R⁸ and R⁹ are independently as defined in the Examples herein.

Another preferred group of compounds are those of the examples below and the salts, solvates and prodrugs thereof.

Particularly preferred compounds include:

### EXAMPLE 4

3-(2,3-Dimethylbenzyl)-1-isobutyl-1-(1-pyridin-2-yl-cyclohexylmethyl)urea

### EXAMPLE 7

Ethyl 1-[3-(2,6-diisopropyl phenyl)-1-(4-hydroxybenzyl)-ureidomethyl]-cyclohexanecarboxylate

### EXAMPLE 16

3-[3-(2,6-Diisopropyl-phenyl)-1-(1-pyridin-2-yl-cyclohexylmethyl)-ureidomethyl]-N-ethyl-benzamide.

### EXAMPLE 39

3-(2,6-Diisopropyl-phenyl)-1-(4-hydroxy-benzyl)-1-(1-pyridin-2-yl-cyclohexylmethyl)-urea

### EXAMPLE 67

1-Cyclopropylmethyl-3-(2,6-diisopropylphenyl)-1-(1-pyridin-2-yl-cyclohexylmethyl)-urea

### EXAMPLE 162

3-(2,6-Diisopropyl-phenyl)-1-(4-hydroxy-benzyl)-1-[1-(1-methyl-1H-imidazol-4-yl)-cyclohexylmethyl]-urea

### EXAMPLE 163

3-(2,6-Diisopropylphenyl)-1-(4-hydroxybenzyl)-1-[1-(2-nitrophenyl)-cyclohexylmethyl)-urea

### EXAMPLE 168

1-[1-(2,6-Difluorophenyl)-cyclohexylmethyl]-3-(2,6-diisopropylphenyl)-1-(4-hydroxybenzyl)-urea

### EXAMPLE 169

3-(2,6-Diisopropylphenyl)-1-(4-hydroxybenzyl)-1-[1-(1-methyl-1H-pyrrol-3-yl)-cyclohexylmethyl)-urea

### EXAMPLE 170

3-(2,6-Diisopropylphenyl)-1-(4-hydroxybenzyl)-1-(1-thiophen-3-yl-cyclohexylmethyl)-urea

### EXAMPLE 177

3-(2,6-Diisopropylphenyl)-1-(4-hydroxybenzyl)-1-[1-(2-methoxyphenyl)-cyclohexylmethyl]urea

### EXAMPLE 187

3-(2,6-Diisopropylphenyl)-1-(4-hydroxybenzyl)-1-(1-thiophen-2-yl-cyclohexylmethy)urea

### EXAMPLE 188

3-(2,6-Diisopropyl phenyl)-1-(4-hydroxybenzyl)-1-[1-(5-methoxy-pyridin-2-yl)-cyclohexylmethy]urea

In another embodiment, the invention provides a class of compounds of formula (I):

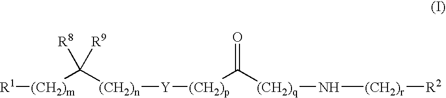

Wherein:

R¹ is selected from

a) aryl

b) aromatic heterocycle

c) CO₂R⁵

d) CONR⁵R⁶

e) NR⁵R⁷

f) OR⁵

g) C₁₋₆ alkyl; and

h) C₁₋₆ cycloalkyl;

Wherein group (a) may be optionally substituted by 1-3 groups each independently selected from NR⁵R⁵, N(R⁵)C(O)R⁵, NO₂, halo, OR⁵, R⁵ and R⁴NR⁵R⁵; and group (b) may be optionally substituted by 1-3 groups each independently selected from halo, R⁵ and OR⁵.

m is 0-2

n is 0-2

p is 0-2

q is 0-2

r is 0-4

Y is NR³ or CHR³

R² is selected from:

a) C₃₋₇ cycloalkyl;

b) aromatic heterocycle, optionally fused with a phenyl group;

c) aryl, wherein said aryl group may optionally be fused with a heterocycle or a C₃₋₇ cycloalkyl group, wherein said fused cycloalkyl moiety may also incorporate a C═O group;

d) Oaryl;

e) C₁₋₆ alkyl, optionally-substituted by OH or Ph;

f) adamantly; and

g) C₁₋₆ alkenyl, optionally substituted by Ph;

wherein groups (a), (b), (c) and (d) may be optionally substituted by 1-3 substituents selected from: R⁵, C₁₋₆ alkenyl, phenyl, OR⁴, OH, CF₃, halo, SO₂R⁵, NO₂, SR⁵, CN, OCF₃, CO₂R⁵, C(O)R⁵, Oaryl, OR⁴aryl, R OR⁵, C(N)NR⁵R⁵, OCOC₁₋₆ alkyl and NR⁵R⁷.

R³ is selected from:

a) C₁₋₆ alkyl;

b) C₁₋₆ alkenyl;

c) C₁₋₆ alkynyl;

d) Aromatic heterocycle, optionally fused with phenyl;

e) Phenyl, optionally fused with phenyl, heterocycle and aromatic heterocycle; said groups (a), (b), (c), (d) and (e) optionally substituted by 1-3 groups each independently selected from halo, CN, SR⁵, heterocycle, aromatic heterocycle, OH, CF3, OR⁵, OR⁵OR⁵, NR⁵R⁷, CO₂H, CO₂R⁵, C₃₋₇ cycloalkyl group (wherein said cycloalkyl group may optionally be substituted by C₁₋₆ alkyl) and phenyl, wherein said phenyl may optionally be fused with a heterocycle, phenyl or C₃₋₇ cycloalkyl, said phenyl or fused phenyl optionally substituted by 1-3 groups each independently selected from: phenyl, R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, NC(O)R⁵, C₁₋₆ alkenyl, OCF₃, NO₂, halo, NSO₂R⁵, SO₂NR⁵R⁵, C(O)NR⁵R⁵, C(N)NR⁵R⁵, OR⁵, OC(O)R⁴-heterocycle and NR⁵R⁷.

R⁴ is C₁₋₆ alkyl.

R⁵ is selected from H and C₁₋₆ alkyl, said alkyl groups optionally substituted by 1-3 groups each independently selected from halo or OH.

R⁶ is selected from H, heterocycle, OC₁₋₆ alkyl, C₁₋₆ alkyl, said alkyl groups optionally substituted by 1-3 groups each independently selected from halo or OH.

R⁷ is selected from H and C₁₋₆ alkyl, said alkyl groups optionally substituted by an aryl group.

R⁸ and R⁹ are both independently selected from H or C₁₋₆ alkyl; or R⁸ and R⁹ may combine to form a 3-7 membered cycloalkyl group. Optionally said cycloalkyl group may incorporate an atom or group selected from NR⁴, NH, O or S.

In another preferred embodiment of the invention:

R¹ is selected from: aryl, aromatic heterocycle, CO₂R⁵, CONR⁵R⁶, NR⁵R⁷ and OR⁵.

More preferably, R¹ is selected from: phenyl, aromatic heterocycle, CO₂R⁵ where R⁵ is Et; and CONR⁵R⁶ where R⁵ and R⁶ combine to form a morpholine ring.

Yet more preferably, R¹ is selected from: aromatic heterocycle and CONR⁵R⁶ where R⁵ and R⁶ combine to form a morpholine ring.

Yet even more preferably R¹ is pyridyl.

Most preferably R¹ is 2-pyridyl.

Preferably m is 0-1, more preferably 0

Preferably n is 0-1, more preferably 1

Preferably p is 0-1, more preferably 0

Preferably q is 0-1, more preferably 0

Preferably r is 0-1, more preferably 0

Preferably Y is NR³

Preferably R² is selected from:

a) C₃₋₇ cycloalkyl;

b) aromatic heterocycle, optionally fused with a phenyl group;

c) aryl, wherein said aryl group may optionally be fused with a heterocycle or a C₃₋₇ cycloalkyl group, wherein said fused cycloalkyl moiety may also incorporate a C═O group;

d) OPh;

e) —CH₂OHCH₂Ph;

f) adamantyl; and

g) —CH═CHPh₂;

wherein groups (a), (b), (c) and (d) may be optionally substituted by 1-3 substituents selected from: C₁₋₆ alkyl, C₁₋₆ alkenyl, phenyl, OR⁴, OH, CF₃, halo, SO₂R⁵, NO₂, SR⁵, CN, OCF₃, CO₂R⁵, C(O)R⁵, Oaryl, OR⁴aryl, R⁴OR⁵, C(N)NR⁵R⁵, OCOC₁₋₁₆ alkyl and NR⁵R⁷.

More preferably R² is a phenyl or naphthalene group, optionally substituted by 1-3 substituents selected from C₁₋₃ alkyl, CF³, halo, OR⁵ and NR⁵R⁷.

Yet more preferably R² is phenyl substituted by 2 substituents selected from C₁₋₃alkyl, halo and NR⁵R⁷.

Most preferably R² is phenyl substituted by 2 substituents selected from Me, chloro, isopropyl and NMe₂. In a particularly preferred embodiment both phenyl substituents are the same.

Preferably R³ is selected from:

a) C₁₋₆ alkyl;

b) C₁₋₆ alkenyl;

c) C₁₋₆ alkynyl;

d) Aromatic heterocycle, optionally fused with phenyl; said aromatic heterocycle or fused heterocycle being optionally substituted by 1-3 substituents each independently selected from: halo, OC(O)CH₃ and —CH₂OC(O)CH₃; and

e) Phenyl, optionally fused with a heterocycle or aromatic heterocycle, said phenyl or fused phenyl optionally substituted by 1-3 substituents each independently selected from: C₁₋₆ alkyl, C(O)NR⁵R⁷, SO₂NR⁵R⁷ and NHSO₂R⁵;

said groups (a), (b) and (c) optionally substituted by 1-3 groups each independently selected from halo, CN, SR⁵, heterocycle, aromatic heterocycle, OH, CF3, OR⁵, OR⁵OR⁵, NR⁵R⁷, CO₂H, CO₂R⁵, C₃₋₇ cycloalkyl group (wherein said cycloalkyl group may optionally be substituted by C₁₋₆ alkyl) and phenyl, wherein said phenyl may optionally be fused with a heterocycle, phenyl or C₃₋₇ cycloalkyl, said phenyl or fused phenyl optionally substituted by 1-3 groups each independently selected from: R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, NC(O)R⁵, C₁₋₆ alkenyl, OCF₃, NO₂, halo, NSO₂R⁵, SO₂NR⁵R⁵, C(O)NR⁵R⁵, C(N)NR⁵R⁵, OR⁵, OC(O)R⁴-heterocycle and NR⁵R⁷.

More preferably R³ is selected from: C₁₋₃ alkyl, optionally substituted by 1-2 groups each independently selected from OH, OR⁵, NR⁵R⁷, CO₂R⁵ and phenyl, wherein said phenyl may optionally be fused with a heterocycle, said phenyl or fused phenyl optionally substituted by 1-3 groups each independently selected from: halo, NO₂, NSO₂R⁵, SO₂NR⁵R⁵, C(O)NR⁵R⁵, C(N)NR⁵R⁵, OR⁵ and NR⁵R⁷.

Yet more preferably R³ is C₁₋₃ alkyl, substituted by phenyl, wherein said phenyl may optionally be fused with a heterocycle, said phenyl or fused phenyl optionally substituted by 1-3 groups each independently selected from: halo, OR⁵ and NO₂.

Most preferably R³ is C₁ alkyl, substituted by phenyl, optionally substituted by 1-3 groups each independently selected from: chloro, OH and NO₂.

Preferably R⁴ is C₁ alkyl.

Preferably R⁵ is H or C₁₋₆ alkyl optionally substituted by OH or trisubstituted by F

More preferably R⁵ is H or C₁₋₃ alkyl optionally substituted by OH or trisubstituted by F.

Preferably R⁷ is H or C₁₋₆ alkyl.

More preferably R⁷ is H or C₁₋₃ alkyl.

Preferably R⁸ and R⁹ combine to form a 3-7 membered cycloalkyl group, wherein said cycloalkyl group may optionally incorporate a heteroatom selected from NH, NR⁴, O or S.

More preferably R⁸ and R⁹ combine to form a 3-7 membered cycloalkyl group.

Most preferably R⁸ and R⁹ combine to form a 6 membered cycloalkyl group.

Preferably aryl is phenyl.

Preferably aromatic heterocycle is a 5 membered ring, containing from 1 to 4 heteroatoms, each independently selected from O, S and N. More Preferably aromatic heterocycle is a 5 membered ring, containing 2 heteroatoms, each independently selected from O, S and N. Most preferably aromatic heterocycle is pyridyl.

Other particularly preferred compounds include:

### EXAMPLE 39

### 3-(2,6-Diisopropyl-phenyl)-1-(4-hydroxy-benzyl)-1-(1-pyridin-2-yl-cyclohexylmethyl)-urea

### EXAMPLE 27

### 3-(2,6-Diisopropyl-phenyl)-1-(3-oxo-3,4-dihydro-2H-benzo[1,4]oxazin-7-ylmethyl)-1-(1-pyridin-2-yl-cyclohexylmethyl)-urea

### EXAMPLE 20

### 3-[3-(2,6-Diisopropyl-phenyl)-1-(1-pyridin-2-yl-cyclohexylmethyl)ureidomethyl]benzamide

### EXAMPLE 21

### 3-[3-(2,6-Diisopropyl-phenyl)-1-(1-pyridin-2-yl-cyclohexylmethyl)-ureidomethyl]-N-methyl-benzamide

### EXAMPLE 16

### 3-[3-(2,6-Diisopropyl-phenyl)-1-(1-pyridin-2-yl-cyclohexylmethyl)-ureidomethyl]-N-ethyl-benzamide

### EXAMPLE 17

### 3-[3-(2,6-Diisopropyl-phenyl)-1-(1-pyridin-2-yl-cyclohexylmethyl)-ureidomethyl]-N-propyl-benzamide.

### EXAMPLE 23

### N-{4-[3-(2,6-Diisopropyl-phenyl)-1-(1-pyridin-2-yl-cyclohexylmethyl)-ureidomethyl]-phenyl}-methanesulfonamide

### EXAMPLE 74

### 3-(2,6-Diisopropyl-phenyl)-1-[3-(2-hydroxy-ethoxy)-benzyl]-1-(1-pyridin-2-yl-cyclohexylmethyl)-urea

### EXAMPLE 28

### 3-(2,6-Diisopropyl-phenyl)-1-(2-oxo-2,3-dihydro-benzooxazol-5-ylmethyl)-1-(1-pyridin-2-yl-cyclohexylmethyl)-urea

### EXAMPLE 37

### 3-(2,6-Bis-dimethylamino-phenyl)-1-(4-hydroxy-benzyl)-1-(1-pyridin-2-yl-cyclohexylmethyl)-urea

### EXAMPLE 38

### 3-(2,6-Diisopropyl-phenyl)-1-(4-hydroxy-benzyl)-1-[1-(2-hydroxy-phenyl)-cyclohexylmethyl]-urea

Another aspect of the invention is a compound of formula (I) described herein, without proviso, including the salts, solvates and prodrugs thereof, for use in medicine.

Another aspect of the invention is a compound of formula (I) described herein, without proviso, including the salts, solvates and prodrugs thereof, for use in the treatment of anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction.

Another aspect of the invention is a compound of formula (I) described herein, without proviso, including the salts, solvates and prodrugs thereof, for the manufacture of a medicament for the treatment of anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction.

Another aspect of the invention is a compound of formula (i) described herein, without proviso, including the salts, solvates and prodrugs thereof, for the manufacture of a medicament for the treatment of for the treatment of male erectile dysfunction and female sexual dysfunction.

Another aspect of the invention is a compound of formula (I) described herein, without proviso, including the salts, solvates and prodrugs thereof, for the manufacture of a medicament for the treatment of for the treatment female sexual arousal dysfunction.

Another aspect of the invention is a pharmaceutical composition comprising a compound of formula (I) described herein, without proviso, including the salts, solvates and prodrugs thereof and a pharmaceutically acceptable diluent, carrier or adjuvant.

Another aspect of the invention is a method of treating anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction comprising administering a therapeutically-effective amount of a compound according to any one of claims 1 to 21.

A further embodiment of the invention provides for compounds useful in the synthesis of compounds of formula (I). Such compounds include compounds of formula (II):

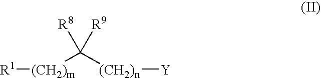

Wherein R¹, R⁸, R⁹, m, n and Y are as herein described.

All the reactions and the preparations of novel starting materials used in the methods herein are conventional and appropriate reagents and reaction conditions for their performance or preparation as well as procedures for isolating the desired products will be well-known to those skilled in the art with reference to literature precedents and the Examples and Preparations hereto.

Compounds of formula (I) where R¹, R², R⁸, R⁹, m, n and r are as described above; Y is NR³, p is 0 and q is 1, may be prepared by the following process as described in scheme (I):

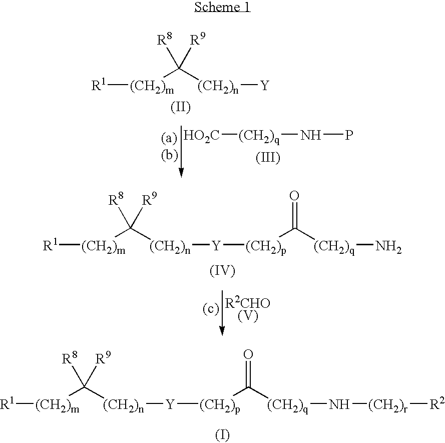

Compounds-of formula (IV) may be prepared by reacting compounds of formula (II) and (III) under the conditions of process step (a) Amide bond formation—such reactions may be carried out under a wide variety of conditions well known to the skilled man.

Typically—The carboxylic acid may be activated by treatment with an agent such as 1,1′-carbonyldiimidazole (CDI), fluoro-N,N,N′,N′-tetramethylformamidinium hexafluorophosphate (TFFH), or a combination of reagents such as azabenzotriazol-1-yloxytris(pyrrolidino)phosphonium hexafluorophosphate (PyAOP) and 1-hydroxy-7-azabenzotriazole (HOAt). Alternatively, the reaction may be carried out by addition of a peptide coupling agent such as O-(7-azabenzotriazol-1-yl)-N,N,N′,N′-uronium hexafluorophosphate (HATU), or O-benzotriazol-1-yl-N,N,N′,N′-uronium hexafluorophosphate (HBTU), or N,N′-dicyclohexylcarbodiimide (DCC), 1,3-diisopropylcarbodiimide (DIC) to a mixture of the acid and amine. The reaction is carried out in a suitable solvent such as CH₂Cl₂, Pyridine, N,N-dimethylformamide (DMF), N,N-dimethylacetamide (DMA) or 1-methyl-2-pyrrolidinone between 0° C. and the boiling point of the solvent.

Preferably 1.25 eq of DIC, 0.125 eq HOBt, 1.1 eq of (III) and 1 eq of (II) in CH₂Cl₂ at RT for 18 h.

The product of process step (a) is then treated under the conditions of process step (b) Deprotection of a nitrogen protecting group P—acid catalysed removal of protecting group using a suitable solvent at RT. A suitable group P includes BOC, CBz or benzyl. Suitable groups are further described in “Protective Groups in Organic Synthesis” by T. W. Greene and P. G. M. Wuts, John Wiley and Sons Inc, 1991.

When P is BOC:

Typically—protected amine and excess HCl at RT for 1-24 h in a solvent such as 1,4-dioxane, ethyl acetate, dichloromethane.

Or, protected amine and an excess of trifluoroacetic acid (TFA) at RT for 1-24 h with or without a solvent such as dichloromethane.

Preferably—the amine was stirred at RT in the presence of an excess anhydrous HCl for 18 h in 1,4-dioxan.

When P is CBz:

Typically—by catalytic hydrogenation in the presence of a suitable catalyst (eg. Pd/C) or by transfer hydrogenation (Pd/C, ammonium formate) in a suitable solvent such as ethanol or methanol, or under acidic conditions such as HBr/acetic acid at room temperature for up to 12 hours.

Preferably Pd/C, in the presence of ammonium formate, in methanol at reflux for 30 minutes.

Compounds of formula (I) may be prepared by reacting compounds of formula (V) and (IV) under the conditions of process step (c) Reductive amination—dehydration of an amine and aldehyde followed by reduction of the formed imine, by a suitable metal hydride reducing agent, in a suitable solvent at RT.

Typically—equimolar amounts of the amine and aldehyde are stirred with sodium triacetoxyborohydride (STAB) or NaBH₃CN at room temperature for 1-24 hours, in CH₂Cl₂, tetrahydrofuran (THF), or (CH₂Cl)₂ optionally in the presence of a drying agent (eg. Molecular sieves), or with removal of water (eg. Using a Dean and Stark apparatus) in a suitable solvent such as toluene.

Or, equimolar amount of the amine and aldehyde are mixed for a time 1-18 h, followed by excess reducing agent, such as NaBH₄, STAB, lithium aluminiumhydride (LAH), in an appropriate solvent such as tetrahydrofuran (THF), Et₂O, MeOH, EtOH.

Preferably −3.5 fold excess of Na(OAc)₃BH(STAB), 1:1 eq of amine:aldehyde in dichloromethane at room temperature for between 0.5 and 12 hours. Alternatively, 1:1 eq amine:aldehyde in toluene at reflux temperature, using Dean and Stark apparatus to remove the water, followed by NaBH₄ in ethanol, at room temperature.

In the synthesis of (I), where R¹ contains a further reactive centre, eg. N atom, a suitable protecting strategy may be employed, for example, the method used in the preparation of Example 179.

Compounds of formula (II) where R¹, R⁸, R⁹, m, n and Y are as described above may be prepared by the following process as described in Scheme 2:

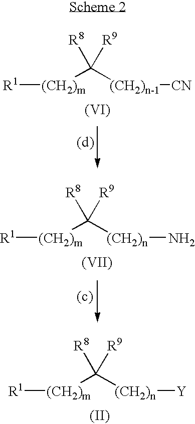

Compounds of formula (VII) may be prepared by reacting compounds of formula (VI) under the conditions of process step (d) Nitrile reduction—reduction of the nitrile group to the analogous amine.

Typically—the nitrile is reduced in a hydrogen atmosphere under pressure 1-20 psi, with a suitable catalyst, in a suitable solvent, such as MeOH, EtOH, optionally including saturated ammonia at RT-100° C. Or, the nitrile is treated with a metal hydride reducing agent, such as LAH, or NaBH₄ with a Lewis acid eg AlCl₃ in a suitable solvent, such as Et₂O, THF, 1,2-dimethoxyethane (DME), at 0-100° C., or by treatment with borane.

Preferably—the nitrile was reduced using catalytic Raney® Ni in saturated ammoniacal ethanol under hydrogenation conditions—30° C., 50 psi and 48 h, or by treatment with 1 eq.LAH in Et₂O at room temperature for up to 1 hour, optionally in the presence of a Lewis acid, preferably 1 eq. AlCl₃. Alternatively, by treatment with borane-methyl sulphide complex in toluene at the reflux temperature of the reaction for about 2 hours.

Compounds of formula (II) may be prepared by reacting compounds of formula (VII) with an appropriate aldehyde derivative of R³, under the conditions of process step (c) as described above.

Preferably—equimolar amount of the amine and aldehyde were stirred in CH₂Cl₂ or (CH₂Cl)₂ for 18 at RT in the presence of 2 equivalents of STAB.

Compounds of formula (II) where R¹, R⁸, R⁹, m, n and Y are as described above may be prepared by the following process as described in Scheme 3:

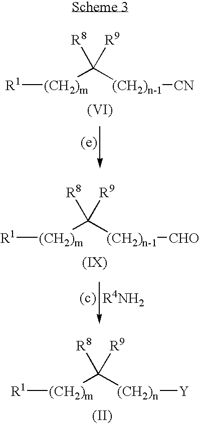

Compounds of formula (VIII) may be prepared by reacting compounds of formula (VI) under the conditions of process step (e) Reduction of nitrile—reduction of the nitrile to the aldehyde.

Typically—the nitrile is treated with a metal hydride, 1-5 equivalents, such as lithium triethoxyaluminium hydride, diisobutylaluminium hydride (DIBAlH), in a solvent such as Et₂O, THF, toluene at −78° C. to RT for 0-6 h, and then subjected to an acidic work-up.

Or, the nitrile is heated RT—reflux temperature of the solvent, in aqueous formic acid with a catalyst such as Raney® Ni for 0-2 h.

Preferentially—the nitrile is heated to 100° C. in aqueous formic acid with a mass equivalent of Raney® Ni for 20 min.

Compounds of formula (II) may be prepared by reacting compounds of formula (VIII) and (IX) under the conditions of process step (c) as described above.

Compounds of formula (I) where R¹, R², R⁸, R⁹, m, n and r are as described above; Y is NR³, p is 0 and q is 1, may be prepared by the following process as described in Scheme 4:

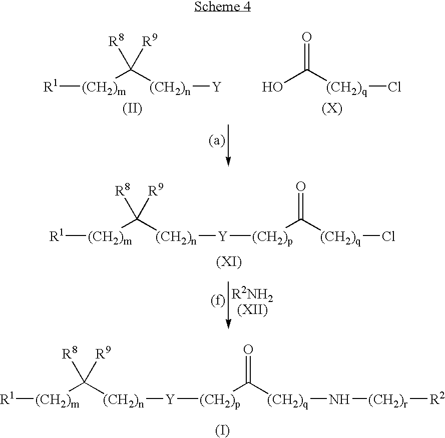

Compounds of formula (XI) may be prepared by reacting compounds of formula (II) with compounds of formula (X) under the conditions of process step (a) Amide bond formation as described above.

Compounds of formula (I) may be prepared by reacting compounds of formula (XII) and (XI) under the conditions of process step (f) Nucleophilic displacement—using amine, suitable base and solvent.

Typically—heating the chloro-acetamide with the amine (XII) in the presence of base, such as N-methylmorpholine (NMM), N,N-diisopropylethylamine (DIPEA), triethylamine (TEA) in a solvent such as DMF, DMA.

Preferably—equimolar amounts of the amine (XII) and chloro-acetamide were heated to 80° C. for 18 h in DMA with 3 equivalents of DIPEA.

Compounds of formula (I) where R¹, R², R⁸, R⁹, m, n and r are as described above; Y is NR³, p is 1 and q is 0, may be prepared by the following process as described in Scheme 5:

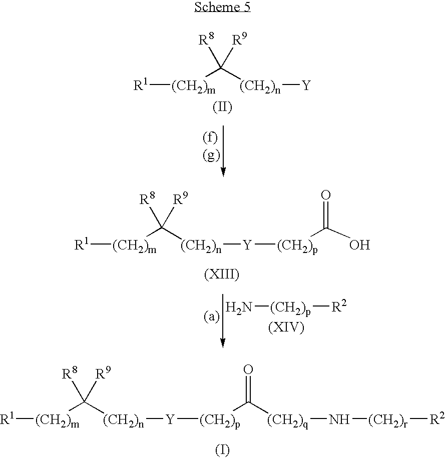

Compounds of formula (XIII) may be prepared by reacting compounds of formula (II) under the conditions of process step (g) Nucleophilic displacement—using amine and suitable base and solvent.

Typically—heating chloroacetic ester with the amine in the presence of base, such as NMM, DIPEA, TEA in a solvent such as DMF, DMA.

Preferably—amine (II) was heated to 75° C. in DMA with a slight excess of DIPEA and a slight excess of methyl chloroacetate for 18 h.

The product of process step (g) is then treated under the conditions of process step (h) Ester hydrolysis—the ester can be treated with either acid or base, optionally with heating in a suitable solvent to effect the hydrolysis.

Typically—the ester is treated with a metal hydroxide (Li, Na, K) in an aqueous solvent eg, MeOH, EtOH, THF or dioxan at the reflux temperature of the solvent.

Preferably—an alcoholic (eg. EtOH, MeOH) solution of the ester was stirred at RT for 12 h in the presence of approximately 2-3 equivalents of aqueous NaOH, or LiOH.

Compounds of formula (I) may be prepared by reacting compounds of formula (XIII) with compounds of formula (XIV) under the conditions of process step (a) as described herein; preferably—equimolar amounts of the acid, HBTU, amine, with 1-3 equivalents of DIPEA in DMF at 50° C. for 18 h.

Compounds of formula (I) where R¹, R², R⁸, R⁹, m, n and r are as described above; Y is NR³, p is 0 and q is 0, may be prepared by the following process as described in Scheme 6:

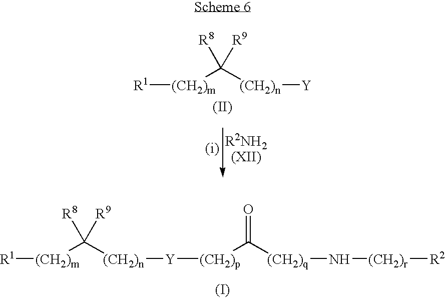

Compounds of formula (I) may be prepared by reacting compounds of formula (II) under the conditions of process step (i) Urea formation 1—activation of (II) and subsequent reaction with amine (XII).

Typically—reacting amine (II) with a reactive carbonyl group, such as phosgene, triphosgene, or p-nitrophenyl chloroformate which generate a reactive intermediate such as the carbamoyl chloride or p-nitrophenylcarbamate, which if R¹ is 2-pyridyl then an intra-molecular reaction can take place giving the pyridinium salt, in a suitable solvent such as CH₂Cl₂, EtOAc, DMF with a base such as NMM, DIPEA, TEA, at −10° C. to RT. The intermediate can then be treated with amine (XII) to give the product.

Preferably—amine (II) is treated, with 0.4 of an equivalent of triphosgene in the presence of 3 equivalents of DIPEA, in CH₂Cl₂ at 0° C. An equivalent of, amine (XII) is then added and the reaction stirred at RT for 6 h.

Compounds of formula (I) where R¹, R², R⁸, R⁹, m, n and r are as described above; Y is NR³, p is 0 and q is 0, may be prepared by the following process as described in Scheme 7:

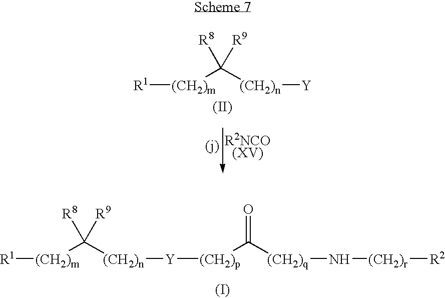

Compounds of formula (I) may be prepared by reacting compounds of formula (II) under the conditions of process step (j). Urea formation 2—treating the isocyanate (XV) with amine (II) in a suitable solvent in the presence of base.

Typically—the isocyanate (XV) is treated with amine (II), optionally in the presence of a co-base such as DIPEA, TEA, Pyridine, NMM in a suitable solvent such as EtOAc, DMF, THF, CH₂Cl₂ at RT for up to 24 h.

Preferentially—the isocyanate (XV) is treated with an equivalent of amine (II) in CH₂Cl₂, DMF or THF at RT for 1-6 h.

R²NCO (XV) may be formed from R²NH₂ (XII) by Isocyanate formation—activation of the amine (XII) with a suitable carbonyl activating group.

Typically—reacting amine (XII) with a reactive carbonyl group, such as phosgene, triphosgene, or p-nitrophenyl chloroformate which can generate the isocyanate in the presence of a base such as NMM, DIPEA, TEA, Pyridine, in a solvent such as CH₂Cl₂, EtOAc, DMF at 0° C. to the reflux temperature of the solvent for up to 24 h.

Preferably—amine (XII) is treated with 0.4 of an equivalent of triphosgene in the presence of 3 equivalents of DIPEA, in CH₂Cl₂ at 0° C. for 10 min

Compounds of formula (I) where R¹, R², R⁸, R⁹, m, n and r are as described above; Y is CHR³, p is 0 and q is 0, may be prepared by the following process as described in Scheme 8:

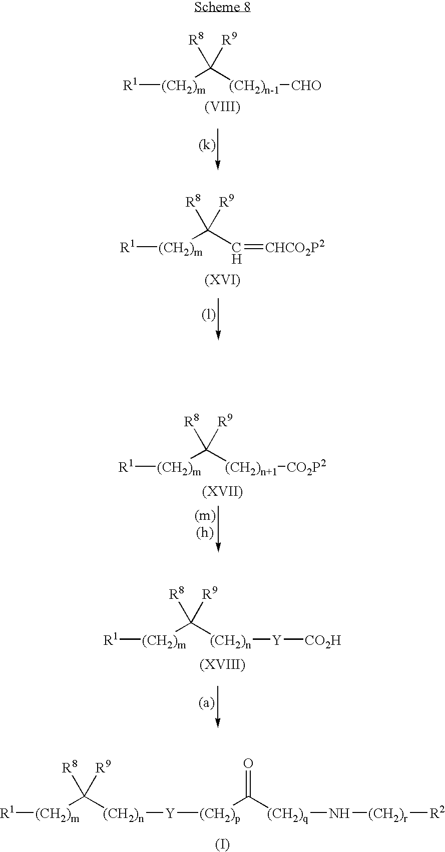

Compounds of formula (XVI) may be prepared by reacting compounds of formula (VIII) under the conditions of process step (k) an olefination reaction.

P² is an ester protecting group, as disclosed in “Protective Groups in Organic Synthesis” by T. W. Greene and P. G. M. Wuts, John Wiley and Sons Inc, 1991. Preferably P² is a C₁₋₆ alkyl group.

Typically—the aldehyde is homologated to the unsaturated ester using Wittig type chemistry (see ‘Advanced Organic Chemistry’ by Jerry March, John Wiley and Sons Inc, 1985). For example, the Wittig reagent in a suitable solvent, such as benzene, toluene, THF, Et₂O or DME, is treated with a slight excess of base, such as BuLi, LDA, MHMDS (M=metal such as Li, Na, K), or NaH at −78° C. to the reflux temperature of the solvent for 0-4 h. Then treated with the aldehyde for 0-6 h at −78° C. to the reflux temperature of the solvent.

Preferentially—1.1 equivalents of NaH is treated with the Wittig reagent (phosphonate ester) in DME for 1 h at RT. An equivalent of the aldehyde is added and the temperature is kept below 30° C. for 15 min.

Compounds of formula (XVII) may be prepared by reacting compounds of formula (XVI) under the conditions of process step (I) Alkene reduction.

Typically—the alkene in an appropriate solvent, such as MeOH, EtOH, EtOAc is catalytically hydrogenated at 15-90 psi, RT to 50° C. in the presence of a catalyst such as palladium on carbon for 1-24 h.

Preferentially—the alkene is subjected to hydrogenation at 50 psi, RT in the presence of a catalytic amount of palladium on carbon for 12 h.

Compounds of formula (XVIII) may be prepared by reacting compounds of formula (XVII) under the conditions of process step (m), Alkylation—the α-methylene is alkylated by deprotonation and electrophile quenching.

Typically—the ester of formula (XVII) is treated with a equivalent of strong base, such as LDA, MHMDS (M is Li, Na, K), BuLi, NaH in a solvent such as THF, Et₂O, DME, at −78° C. to 0° C. for 0-4 h. The electrophile (R³Br), is added and the reaction mixture is warmed to RT.

Preferentially—the ester of formula (XVII) in DME at −50° C. is treated with an equivalent of LiHMDS, for 1 h, the electrophile (R³Br), is added at −78° C. and the mixture allowed to warm to RT over 1 h.

The product of process step (m) is then treated under the conditions of process step (h) ester hydrolysis (removal of P²) as described herein, to provide the acid of formula (XVIII).

Preferentially—the ester was stirred at 70° C. for 3 days in a 3:1 mixture of dioxan:water in the presence of 2.5 equivalents of LiOH.

Compounds of formula (I) may be prepared by reacting compound of formula (XVIII) under the conditions of process step (a) as described herein.

Compounds of formula (II) wherein R¹ represents CONR⁵R⁶ may be prepared by the process as described in Scheme 9:

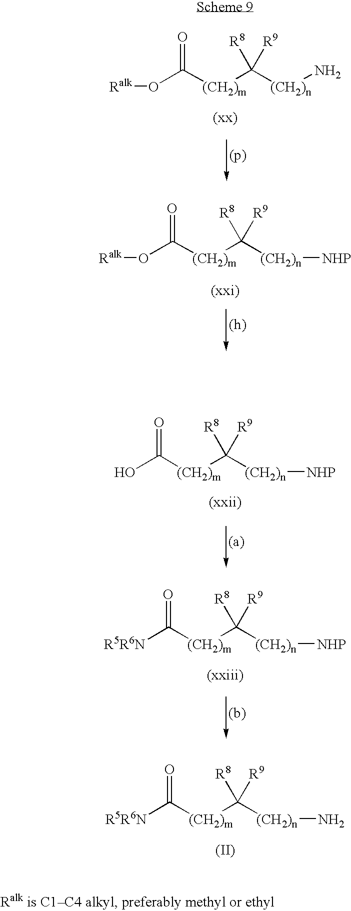

Compounds of formula (II) where R¹ represents CONR⁵R⁶ may be prepared according to scheme 9 by reacting compounds of formula (xx) under the conditions of process step (p) Protection of a reactive N atom. Such reactions may be carried out under a variety of conditions well known to the skilled man.

Typically—Protection of a reactive N atom may be carried out using a suitable protecting group P, typically BOC, CBz, benzyl, but preferably BOC or CBz under standard conditions as described in “Protective groups in Organic Synthesis” by T. W. Greene and P. G. M. Wuts, John Wiley and Sons Inc, 1991.

Preferably—1.1 eq (BOC)₂O, optionally in the presence of a base (eg. Na₂CO₃) in dioxan and water, at room temperature, for about 5 hours. Alternatively, 1.1 eq benzyl chloroformate, in the presence of a base (eg. K₂CO₃) in dioxan in dioxan and water at room temperature for up to an hour.

Process steps (h), (a) and (b) may be carried out as herein described.

Compounds of formula (II) where Y represents NR³ and n=1 may be prepared according to scheme 10.

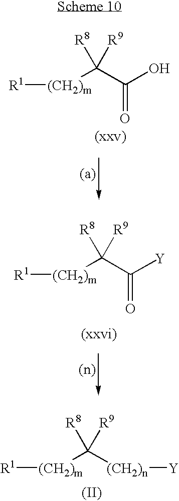

Step (a) may be carried out according to the methods as herein described.

Preferably, reaction step (a) is carried out using DCC and pentafluorophenol (1:1 eq) combination, in ethyl acetate.

Step (n)—Reduction of amide (xxvi) to the amine of formula (II) may be achieved by treatment with a suitable metal hydride reducing agent (eg. LiAlH₄), or by treatment with borane.

Preferably, the reduction is achieved using a borane-methyl sulphide complex, in THF at reflux temperature for 16 hours.

Unless otherwise provided herein:

WSCDI means 1-(3-dimethylaminopropyl)-3-ethylcarbodiimide hydrochloride;

DCC means N,N′-dicyclohexylcarbodiimide;

HOAT means 1-hydroxy-7-azabenzotriazole;

HOBT means 1-hydroxybenzotriazole hydrate;

PyBOP® means Benzotriazol-1-yloxytris(pyrrolidino)phosphonium hexafluorophosphate;

PyBrOP® means bromo-tris-pyrrolidino-phosphonium hexafluorophosphate;

Mukaiyama's reagent means 2-chloro-1-methylpyridinium iodide;

HATU means O-(7-azabenzotriazol-1-yl)-N,N,N′N′-tetramethyluroniumhexafluorophosphate

KHMDS means potassium bis(trimethylsilyl)amide;

LDA means lithium diisopropylamide

TEA means triethylamine;

NMM means N-methylmorpholine;

DIPEA means N-ethyldiisopropylamine

DEAD means diethyl azodicarboxylate;

DIAD means diisopropyl azodicarboxylate;

DIBAL-H means diisobutylaluminium hydride;

STAB means sodium triacetoxyborohydride

Dba means dibenzylideneacetone;

Boc means tert-butoxycarbonyl;

CBz means benzyloxycarbonyl;

(Boc)₂O means di-tert-butyl dicarbonate;

CDI means carbonyl diimidazole;

MeOH means methanol, EtOH means ethanol, and EtOAc means ethyl acetate, DME means 1,2-dimethoxyethane.

THF means tetrahydrofuran, DMSO means dimethyl sulphoxide, and DCM means dichloromethane;

AcOH means acetic acid, TFA means trifluoroacetic acid;

Ph means phenyl.

Bombesin antagonists are known for their use in treating disease. In one embodiment, the present invention provides for compounds of formula (I) for use as a medicament.

The present invention also provides for the use of a compound of formula (I) without proviso in the preparation of a medicament for the treatment of anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction

Preferred conditions include male erectile dysfunction and female sexual dysfunction, particularly female sexual arousal dysfunction.

It is to be appreciated that all references herein to treatment include curative, palliative and prophylactic treatment.

Sexual dysfunction (SD) is a significant clinical problem, which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994—Male Erectile dysfunction assessment and treatment options. Comp. Ther. 20: 669-673.).

The compounds of the invention are particularly beneficial for the prophylaxis and/or treatment of sexual dysfunction in the male (e.g. male erectile dysfunction—MED) and in the female—female sexual dysfunction (FSD), e.g. female sexual arousal disorder (FSAD).

Male sexual dysfunction includes male erectile dysfunction, ejaculatory disorders such as premature ejaculation (PE), anorgasmia (inability to achieve orgasm) and desire disorders such as hypoactive sexual desire disorder (lack of interest in sex).

It is known that some individuals can suffer from male erectile dysfunction (MED).

MED is defined as:

“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman, A. & Gingell, J. C. (1999). The epidemiology and pathophysiology of erectile dysfunction. J. Urology 161: 5-11). The condition has a significant negative impact on the quality of life of the patient and their partner, often resulting in increased anxiety and tension which leads to depression and low self esteem. Whereas two decades ago, MED was primarily considered to be a psychological disorder (Benet, A. E. et al (1994), Male erectile dysfunction assessment and treatment options. Comp. Ther. 20: 669-673), it is now known that for the majority of patients there is an underlying organic cause. As a result, much progress has been made in identifying the mechanism of normal penile erection and the pathophysiology of MED.

Penile erection is a haemodynamic event which is dependent upon the balance of contraction and relaxation of the corpus cavernosal smooth muscle and vasculature of the penis (Lerner, S. E. et al (1993). A review of erectile dysfunction: new insights and more questions. J. Urology 149: 1246-1255). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in blood pressure which results in an erection (Naylor, A. M. (1998). Endogenous neurotransmitters mediating penile erection. Br. J. Urology 81: 424-431).

The changes that occur during the erectile process are complex and require a high degree of coordinated control involving the peripheral and central nervous systems, and the endocrine system (Naylor, 1998). Corporal smooth muscle contraction is modulated by sympathetic noradrenergic innervation via activation of postsynaptic α₁ adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than NO, such as calcitonin gene related peptide (CGRP) and vasoactive intestinal peptide (VIP). The main relaxing factor responsible for mediating this relaxation is nitric oxide (NO), which is synthesised from L-arginine by nitric oxide synthase (NOS) (Taub, H. C. et al (1993). Relationship between contraction and relaxation in human and rabbit corpus cavernosum. Urology 42: 698-704). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels).

MED patient groups, which are described in more detail in Clinical Andrology vol 23, no.4, p773-782, and chapter 3 of the book by 1. Eardley and K. Sethia “Erectile Dysfunction—Current Investigation and Management, published by Mosby-Wolfe, are as follows: psyhcogenic, endocrinologic, neurogenic, arteriogenic, drug-induced sexual dysfunction (lactogenic) and sexual dysfunction related to cavernosal factors, particularly venogenic causes. In accordance with the invention, FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders, Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a Woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants eg SSRIs or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

Since interest is relatively recent in treating FSD pharmacologically, therapy consists of the following:—psychological counselling, over-the-counter sexual lubricants, and investigational candidates, including drugs approved for other conditions. These medications consist of hormonal agents, either testosterone or combinations of oestrogen and testosterone and more recently vascular drugs, that have proved effective in male erectile dysfunction. None of these agents has been demonstrated to be very effective in treating FSD.

As discussed, the compounds of the invention are particularly useful for the treatment of female sexual arousal disorder (FSAD).

The Diagnostic and Statistical Manual (DSM) IV of the American Psychiatric Association defines Female Sexual Arousal Disorder (FSAD) as being:

“a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post menopausal (±HRT) women. It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90,1998) with animal data supporting this view (Park et al., Int. J. Impot. Res., 9, 27-37, 1997).

Drug candidates for treating FSAD, which are under investigation for efficacy, are primarily erectile dysfunction therapies that promote circulation to the male genitalia. They consist of two types of formulation, oral or sublingual medications (Apomorphine, Phentolamine, phosphodiesterase type 5 (PDE5) inhibitors e.g. Sildenafil), and prostaglandin (PGE₁) that are injected or administered transurethrally in men, and topically to the genitalia in women.

The compounds of the invention find application in the following sub-populations of patients with FSD: the young, the elderly, pre-menopausal, peri-menopausal, post-menopausal women with or without hormone replacement therapy.

The compounds of the invention find application in patients with FSD arising from:—

i) Vasculogenic etiologies eg cardiovascular or atherosclerotic diseases, hypercholesterolemia, cigarette smoking, diabetes, hypertension, radiation and perineal trauma, traumatic injury to the iliohypogastric pudendal vacular system.

ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.

iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin eg hyperprolactinemia, natural menopause, premature ovarian failure, hyper and hypothyroidism.

iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or a traumatic past experiences.

v) Drug-induced sexual dysfunction resulting from therapy with selective serotonin reuptake inhibitors (SSRis) and other antidepressant therapies (tricyclics and major tranquillizers), anti-hypertensive therapies, sympatholytic drugs, chronic oral contraceptive pill therapy.

A further aspect of the invention provides for the compounds of formula (I) to be coadministered simultaneously, separately or sequentially with one or more therapeutically active agents. Suitable coadministrants include:

(1) One or more naturally occurring or synthetic prostaglandins or esters thereof. Suitable prostaglandins for use herein include compounds such as alprostadil, prostaglandin E₁, prostaglandin E₀, 13,14-dihydroprostaglandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁ α, 19-hydroxy PGA₁, 19-hydroxy-PGB₁, PGE₂, PGB₂, 19-hydroxy-PGA₂, 19-hydroxy-PGB₂, PGE₃α, carboprost, tromethamine, dinoprost, dinoprostone, iloprost, gemeprost, metenoprost, suiprostune, tiaprost and moxisylate.

(2) One or more α-adrenergic receptor antagonist compounds also known as α-adrenoceptor antagonists or α-receptor antagonists or α-blockers. Suitable compounds for use herein include: the α-adrenergic receptor blockers as described in PCT application WO99/30697 published on Jun. 14, 1998, the disclosure of which relating to α-adrenergic receptors incorporated herein by reference and include, selective α₁-adrenoceptor or α₂-adrenoceptor blockers and non-selective adrenoceptor blockers, Suitable α₁-adrenoceptor blockers include: phentolamine, phentolamine mesylate, trazodone, alfuzosin, indoramin, naftopidil, tamsulosin, dapiprazole, phenoxybenzamine, idazoxan, efarxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenamine, tolazoline, trimazosin and dibenamine; α-adrenergic receptor antagonists as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cardiotonic agent such as pirxamine.

(3) One or more NO-donor (NO-agonist) compounds. Suitable NO-donor compounds for use herein include organic nitrates, such as mono- di or tri-nitrates or organic nitrate esters including glyceryl tririnitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine, molsidomine, S-nitroso-N-acetyl penicillamine (SNAP) S-nitroso-N-glutathione (SNO-GLU), N—hydroxy—L-arginine, amylnitrate, linsidomine, linsidomine hydrochloride, (SIN-1) S-nitroso—N-cysteine, diazenium diolates,(NONOates), 1,5-pentanedinitrate, L-arginine, ginseng, zizphi fructus, molsidomine, Re-2047, nitrosylated maxisylyte derivatives such as NMI-678-11 and NMI-937 as described in published PCT application WO 0012075; and/or

(4) One or more potassium channel openers or modulators. Suitable potassium channel openers/modulators for use herein include nicorandil, cromokalim, levcromakalim (lemakalim), pinacidil, diazoxide, minoxidil, charybdotoxin, glyburide, 4-aminopyridine, BaCl₂.

(5) One or more dopaminergic agents, preferably apomorphine or a selective D2, D3 or D2/D₃agonist such as, pramipexole and ropirinol (as claimed in WO-0023056), PNU95666 (as claimed in WO-0040226).

(6) One or more vasodilator agents. Suitable vasodilator agents for use herein include nimodepine, pinacidil, cyclandelate, isoxsuprine, chloropromazine, halo peridol, Rec 15/2739, trazodone.

(7) One or more thromboxane A2 agonists.

(8) One or more CNS active agents.

(9) One or more ergot alkaloids. Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride.

(10) One or more compounds which modulate the action of natriuretic factors in particular atrial natriuretic factor (also known as atrial natriuretic peptide), B type and C type natriuretic factors such as inhibitors or neutral endopeptidase.

(11) One or more compounds which inhibit angiotensin-converting enzyme such as enalapril, and dual inhibitors of both angiotensin-converting enzyme and neutral endopeptidase such as omapatrilat.

(12) One or more angiotensin receptor antagonists such as losartan.

(13) One or more substrates for NO-synthase, such as L-arginine.

(14) One or more calcium channel blockers such as amlodipine.

(15) One or more antagonists of endothelin receptors and inhibitors, of endothelin-converting enzyme.

16) One or more cholesterol lowering agents such as statins (e.g. atorvastatin/Lipitor—trade mark) and fibrates.

(17) One or more antiplatelet and antithrombotic agents, e.g. tPA, uPA, warfarin, hirudin and other thrombin inhibitors, heparin, thromboplastin activating factor inhibitors.

(18) One or more insulin sensitising agents such as triglitazone (rezulin) and hypoglycaemic agents such as glipizide.

(19) L-DOPA or carbidopa.

(20) One or more acetylcholinesterase inhibitors such as donepezil (Aricept).

(21) One or more steroidal or non-steroidal anti-inflammatory agents.

(22) One or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

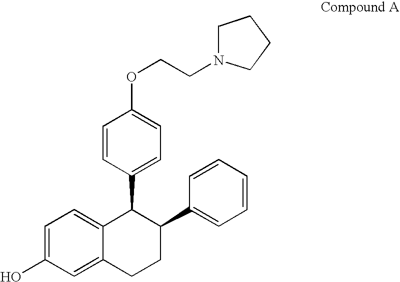

(23) One or more PDE inhibitors, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM.

(24) In the case where the combination is for the treatment or prophylaxis of female sexual dysfunction, one or more of an NPY (neuropeptide Y) inhibitor, more particularly NPY1 or NPY5 inhibitor, preferably NPY1 inhibitor. Preferably said NPY inhibitors (including NPYY1 and NPYY5) have an IC50 of less than 100 nM, more preferably less than 50 nM.

(25) One or more of a NEP inhibitor preferably having an IC50 for NEP of less than 300 nM, more preferably less than 100 nM.

(26) One or more of vasoactive intestinal protein (VIP), VIP mimetic, VIP analogue, more particularly acting through one or more of the VIP receptor subtypes VPAC1, VPAC or PACAP (pituitory adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil).

(27) One or more of a melanocortin receptor agonist or modulator or melanocortin enhancer, such as, melanotan II, PT-14, PT-141 or compounds claimed in WO-09964002, WO-00074679, WO-09955679, WO-00105401,, WO-00058361, WO-00114879, WO-00113112, WO-09954358.

(28) One or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993.

(29) One or more of a testosterone replacement agent (including dehydroandrostendione), testosterone (Tostrelle), dihydrotestosterone or a testosterone implant.

(30) One or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone).

(31) One or more of a modulator of transporters for noradrenaline, dopamine and/or serotonin, such as bupropion, GW-320659.

(32) One or more of a purinergic receptor agonist and/or modulator.

(33) One or more of a neurokinin (NK) receptor antagonist, including those described in WO-09964008.

(34) One or more of an opioid receptor agonist, antagonist or modulator, preferably agonists for the ORL-1 receptor.

(35) One or more of an agonist or modulator for oxytocin/vasopressin receptors, preferably a selective oxytocin agonist or modulator.

(36) One or more modulators of cannabinoid receptors.

According to another preferred aspect of the present invention, there is provided use of a compound of formula (I) and one or more additional active agents for the treatment of female sexual dysfunction (FSD).

Preferably, said one or more additional active agents is/are selected from the group consisting of:

1) estrogen receptor modulators and/or estrogen agonists and/or estrogen antagonists;

2) testosterone replacement agent and/or testosternone (Tostrelle) and/or dihydrotestosterone and/or dehydroepiandrosterone (DHEA) and/or a testosterone implant;

1) estrogen, estrogen and medroxyprogesterone or medroxyprogesterone acetate (MPA) (as a combination), or estrogen and methyl testosterone hormone replacement therapy agent;

2) one or more dopaminergic agents;

3) one or more of an NPY (neuropeptide Y) inhibitor;

4) one or more of a melanocortin receptor agonist or modulator or melanocortin enhancer;

5) one or more of an NEP (neutral endopeptidase) inhibitor; one or more of a PDE (phosphodiesterase) inhibitor;

The present invention provides for a composition comprising a compound of formula (I) and a pharmaceutically acceptable diluent or carrier.

The present invention provides for a composition comprising a compound of formula (I) and one or more additional active agents as described in 1-36 herein and a pharmaceutically acceptable diluent or carrier.

A further embodiment of the invention provides for a kit comprising

a) A pharmaceutical composition comprising a compound of formula (I) and a pharmaceutically acceptable diluent or carrier;

b) A pharmaceutical composition comprising an additional active agent as described in 1-36 herein and a pharmaceutically acceptable diluent or carrier;

for simultaneous, separate or sequential administration.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclose din EP-A-1092719.

Further suitable PDE5 inhibitors for the use according to the present invention include: 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]su lphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4′,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8; 2-[2-ethoxy-5-(4-ethylpiperazin-1-yl-1-sulphonyl)-phenyl]-5-methyl-7-propyl-3H-imidazo[5, 1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example 11 of published international application WO93/07124 (EISAI); and compounds 3 and 14 from Rotella D P, J. Med. Chem., 2000, 43, 1257.

Still other suitable PDE5 inhibitors include: 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furaziocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

Preferred herein are NEP inhibitors wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, candoxatril, candoxatrilat, sampatrilat). Suitable NEP inhibitor compounds are described in EP-A1097719.

Particularly preferred NEPi compounds for as auxiliary agents for use in the treatment of MED according to the present invention are those described in co-pending International Patent Application PCT/IB02/00807 filed on the Mar. 18, 2002.

Especially preferred is (S)-2-[(1-{[3-(4-chlorophenyl)propyl]carbamoyl}cyclopentyl)methyl]-4-methoxybutanoic acid or a pharmacuetically acceptable salt such as the sodium salt thereof as detailed at Example 22 in PCT/IB02/00807. Details for the synthesis of this compound and the sodium salt are provided in the Experimental Section hereinafter.

The compounds of the present invention are a potent class of bombesin antagonists. Bombesin antagonism may be measured using the following binding assay using membranes from CHO cells expressing the human bombesin BB1 receptors.

Experimental Procedure:

Assay Buffer Composition

| 50 mM HEPES HCl, | |
|---|---|
| pH 7.4 at 21° C. | [frozen stock] = |
| containing | 1000× (add 1 μl/ml buffer) |
| 0.02% BSA | 200 mg/ml in H₂O |
| 40 μg/ml bacitracin | 40 mg/ml in H₂O |
| 2 μg/ml chymostatin | 2 mg/ml in DMSO |
| 2 μM phosphoramidon | 2 mM in H₂O |
| 4 μg/ml leupeptin | 4 mg/ml in H₂O |

Cell Culture

Cells are maintained in Ham's F12 medium (Life Technologies Ltd, Cat No. 31765-027) supplemented with 10% FBS (Life Tech. Ltd., 10109-155) and 2 mM glutamine (or Glutamax in media). Cells are routinely passaged once weekly (split 1:5, approximate seeding density=1-4 million per 175 mm² flask), and fed every 2 days with fresh medium. Cells are frozen down (in Hams F12 containing 5% DMSO) 4-7 days after passaging and stored at −70° C. until required for use in binding experiments.

Membrane Preparation

Frozen cells rapidly thawed and diluted with excess medium (each aliquot made up to 40 ml).

Cells harvested by centrifuging at 3000 g for 4 min at 21° C.

Cells resuspended in known volume of assay buffer, and cell count performed.

Membranes prepared by polytronning (setting 5, 10 secs).

Centrifuged at 30,000 g for 10 mins at 21° C.

Pellet resuspended in appropriate volume of assay buffer to add 3×10⁴ cells/250 μl assay.

Cell concentration recently (1998) adjusted to 0.05×10⁶ cells/250 μl assay (stock required=0.25×10⁶ cells/ml).

Membranes used immediately in assay.

Assay Setup

Drug dilutions are performed using the Tecan Genesis/Miniprep handling stations. Assays are set up as follows

25 μl [¹²⁵I][6-14]Bombesin (0.05-0.1 nM final concentration; NEN 377)

25 μl test compound/total/NSB

200 μl cell memb preparation

Assay additions using deep well Multidrop.

Total volume=250 μl.

Non-specific binding is defined by 1 μM Bombesin 1% DMSO (final concentration).

1. Add membranes to start incubation. Vortex and incubate at 21° C. GRP assay=90 min; NMB assay=60 min.

2. Terminate reactions by rapid filtration onto GF\C filters or GF\C Unifilter plates presoaked in 0.2% PEI for >1 hour) using the appropriate Brandel cell harvester.

3. Wash with ice-cold HEPES HCl (50 mM, pH 7.4 at 21° C.).

Filter mats 6×1 ml total wash volume

Unifilter plates 2×1 ml total wash volume

4. Unifilters are dried at 50° C. for 60 minutes. Add 50 μl Microscint-0, Seal plates with Top-Seal A and count using TopCount NXT.

For each assay plate, total (1% DMSO) and non-specific (1 μM, 1% DMSO), binding will be measured for the ligand on the receptor. Specific binding of the radioactive ligand to the receptor can thus be calculated to which specific binding of the radioactive ligand in the presence of a competing compound can be calculated and expressed as percentage inhibition of radioactive ligand binding.

The compounds of the present invention have been found to be potent bombesin antagonists with a Ki of <1000 nM

Moreover the compounds of the present invention have 10 fold selectivity for the BB1 receptor over the BB2.

The compound of example 1 has a BB1 Ki of 82 nM and a Ki for BB2 of 3590 nM

Treatment of MED

Compounds of formula (I) can be screened for effect of penile intracavernosal pressure (ICP) in the conscious male rat according to the methods described herein.

ICP Protocol

Intra cavernosal pressure (ICP) can be measured in the conscious rat by means of telemetric recording. A catheter is surgically implanted into the corpus cavernosum. The end of the catheter is linked to a device, which senses, processes, and transmits information digitally from within the animal. A receiver converts the radio-frequency signal from the implant to a digital pulse stream that is readable by a data collection system. The PC-based system collects telemetred data from the animal.

Surgery:—Induce and maintain general anaesthesia using 5% Isofluranee in a carrier gas of 0.5L/min oxygen and 1L/min nitrous oxide to induce anaesthesia, reducing to 2% Isoflurane for maintenance anaesthesia. Administer 5 mg/kg sub cutaneously (s.c.) Carprofen (Rimadyl® Large Animal Injection, 50 mg/ml, Pfizer Animal Health) at induction of anaesthesia, at end of day of surgery and on the morning of first day post-surgery to minimise pain and discomfort.

Implantation of corpus cavernosal probe:—Shave the skin of the ventral abdomen and extend to include the area around the penis and ventral scrotum. Clean and disinfect the shaved area. Place the rat in dorsal recumbency. Make a mid-line incision from the external base of the penis, running caudally for approximately 2 cm. Locate and expose the internal structure of the penis and identify the corpus cavernosum. Make a mid-line laparotomy, approximately 4 cm in length to access the abdominal cavity. Pierce the abdominal wall via the caudal incision with a suitable trocar and cannula, taking care not to damage any internal organs. Place the implant body in the abdominal cavity with the catheter orientated caudally and pass the catheter tip through the body wall via the preplaced cannula. Implant used is model TA11PA-C40, 8 mm catheter, with modified 3 mm tip (Data Sciences International Inc.). Secure the implant body to the abdominal wall using non-absorbable sutures and partially close the abdominal incision. Reflect the tip of the penis cranially and retract the caudal incision to optimise the surgical field. Carefully isolate approximately 10 mm of the internal structure of the penis from the surrounding tissue. Carefully reflect the corpus spongiosum to one side to give access to the corpus cavernosum. Access the corpus cavernosum using a modified over-the-needle catheter to puncture the tunica. Introduce the catheter tip via the preplaced catheter and advance until fully inserted. Carefully remove the access catheter and apply a suitable tissue adhesive to the insertion site. Observe for leakage. Close the subcutaneous fat layer in the caudal incision before closing with an appropriate absorbable suture. Instil approximately 5 ml of warm saline through the abdominal incision and complete closure of the mid-line incision. Close the skin incision with an appropriate absorbable suture.

Postoperative care:—Measure food and water intake and monitor bodyweight daily for at least 7 days post surgery, then 2-3 times weekly. Give Lectade® (Pfizer Animal Health) in drinking water for 3 days post surgery. House rats singly, and transfer to reverse light/dark conditions 5 days post surgery. Named Veterinary Surgeon (or Deputy) to issue a certificate of fitness to continue 2 days post surgery. Start using rats experimentally 7 days post surgery.

Experimental Procedure:—Perform experiment in room with reverse light/dark conditions. On day of experiment, place rat in home cage on receiver pad (PhysioTel® Model RPC-1, Data Sciences International Inc.) and leave to acclimatise for approximately one hour. Ensure that the rat has food and water ad lib. Take baseline reading of intra cavernosal pressure (ICP) for approximately 5 minutes. Transfer the data via a floppy disk to an Excel spreadsheet. Inject the rat with compound subcutaneously or via the jugular vein catheter (JVC). If, using the JVC, flush through with sterile saline after dosing and seal with a saline/glucose lock solution. The interval between administration of compound and ICP measurement will vary with the compound to be tested. An interval of 30-60 min post s.c. injection is a good guide. The test compounds were dissolved in 50% β-cyclodextrin in saline. They were administered at a dose of 5-10 mg/kg subcutaneously (s.c.). Apomorphine hydrochloride hemihydrate (Sigma A-4393) at 60 μg/kg s.c. was used as a positive control as it has pro-erectile properties. Record ICP over a 15 minute period, starting at 30 minutes post injection i.e. from 30 to 35 minutes and repeat for two further 15 minute periods commencing at 60 minutes post injection and 120 minutes post injection respectively. Record ICP for 15 minutes. A signal from the receiver pad feeds through to the Data Exchange Matrix® and hence to the software (Dataquest ART® acquisition system, Data Sciences International Inc.). Transfer the data via a floppy disk to an Excel spreadsheet for analysis.

Compounds of Formula (I) in Combination With PDE5i for Treatment of MED

The effects of concomitant administration of compound(s) of formula (I) in combination with a PDE5 inhibitor on the penile intracavernosal pressure (ICP) in an anaesthetised rabbit model of erection can be measured according to the following protocol.

Experimental Protocol

Male New Zealand rabbits (˜2.5 kg) are pre-medicated with a combination of Medetomidine (Domitor®) 0.5 ml/kg inramuscularly (i.m.), and Ketamine (Vetalar®) 0.25 ml/kg i.m. whilst maintaining oxygen intake via a face mask. The rabbits are tracheotomised using a Portex™ uncuffed endotracheal tube 3 ID (internal diameter), connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane® and ventilation continued with O₂ at 2 litres/min. The right marginal ear vein was cannulated using a 23G or 24G catheter, and Lactated Ringer solution perfused at 0.5 ml/min. The rabbit was maintained at 3% Isoflurane during invasive surgery, dropping to 2% for maintenance anaesthesia. The left jugular vein was exposed, isolated and then cannulated with a PVC catheter (17 gauge/17G) for the infusion of drugs and the test compounds.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic x2+systolic)÷3. Heart rate was measured via the pulse oxymeter and Po-ne-mah data acquisition software system (Ponemah Physiology Platform, Gould Instrument Systems Inc).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in intracavernosal pressure and cavernosal blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:—5V, pulse width 0.5 ms, duration of stimulus 20 seconds with a frequency of 16 Hz. Reproducible responses were obtained when the nerve was stimulated every 15-20 minutes. Several stimulations using the above parameters were performed to establish a mean control response. The compound(s) to be tested were infused, via the jugular vein, using a Harvard 22 infusion pump allowing a continuous 15 minute stimulation cycle. The skin and connective tissue around the penis was removed to expose the penis. A catheter set (Insyte-W, Becton-Dickinson 20 Gauge 1.1×48 mm) was inserted through the tunica albica into the left corpus cavernosal space and the needle removed, leaving a flexible catheter. This catheter was linked via a pressure transducer (Ohmeda 5299-04) to a Gould system to record intracavernosal pressure (ICP). Once an intracavernosal pressure was established, the catheter was sealed in place using Vetbond (tissue adhesive, 3M). Heart rate was measured via the pulse oxymeter and Po-ne-mah data acquisition software system (Ponemah Physiology Platform, Gould Instrument Systems Inc).

Intracavernosal blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue).

Treatment of FSAD

Serotonin 5HT2C receptor agonists potentiate pelvic nerve-stimulated increases in female genital blood flow in the anaesthetised rabbit model of sexual arousal.

The normal sexual arousal response consists of a number of physiological responses that are observed during sexual excitement. These changes such as vaginal, labial and clitoral engorgement result from increases in genital blood flow. Engorgement leads to increased vaginal lubrication via plasma transudation, increased vaginal compliance (relaxation of vaginal smooth muscle) and increases in vaginal and clitoral sensitivity.

Female sexual arousal disorder (FSAD) is a highly prevalent sexual disorder affecting up to 40% of pre-, per- and postmenopausal (±HRT) women. The primary consequence of FSAD is reduced genital engorgement or swelling which manifests itself as a lack of vaginal lubrication and a lack of pleasurable genital sensation. Secondary consequences include reduced sexual desire, pain during intercourse and difficulty in achieving orgasm. The most common cause of FSAD is decreased genital blood flow resulting in reduced vaginal, labial and clitoral engorgement (Berman, J., Goldstein, I., Werbin, T. et al. (1999a). Double blind placebo controlled study with crossover to assess effect of sildenafil on physiological parameters of the female sexual response. J. Urol., 161, 805; Goldstein, I. & Berman, J. R. (1998). Vasculogenic female sexual dysfunction: vaginal engorgement and clitoral erectile insufficiency syndromes. Int. J. Impot. Res., 10, S84-S90; Park, K., Goldstein, I., Andry, C., et al. (1997). Vasculogenic female sexual dysfunction: The hemodynamic basis for vaginal engorgement insufficiency and clitoral erectile insufficiency. Int. J. Impotence Res., 9, 27-37; Werbin, T., Salimpour, P., Berman, L., et al. (1999). Effect of sexual stimulation and age on genital blood flow in women with sexual stimulation. J. Urol., 161, 688).

As explained herein, the present invention provides a means for restoring or potentiating the normal sexual arousal response in women suffering from FSAD, by enhancing genital blood flow.

Method

Female New Zealand rabbits (˜2.5 kg) were pre-medicated with a combination of Medetomidine (Domitor®) 0.5 ml/kg intramuscularly (i.m.), and Ketamine (Vetalar®) 0.25 ml/kg i.m. whilst maintaining oxygen intake via a face mask. The rabbits were tracheotomised using a Portex™ uncuffed endotracheal tube 3 ID (internal diameter), connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane® and ventilation continued with O₂ at 2l/min. The right marginal ear vein was cannulated using a 23G or 24G catheter, and Lactated Ringer solution perfused at 0.5 m[/min. The rabbit was maintained at 3% Isoflurane® during invasive surgery, dropping to 2% for maintenance anaesthesia.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 10 cm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic x2+systolic)÷3. Heart rate was measured via the pulse oxymeter and Po-ne-mah data acquisition software system (Ponemah Physiology Platform, Gould Instrument Systems Inc).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery, which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in vaginal and clitoral blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:—5V, pulse width 0.5 ms, duration of stimulus 10 seconds and a frequency range of 2 to 16 Hz. Reproducible responses were obtained when the nerve was stimulated every 15-20 minutes. A frequency response curve was determined at the start of each experiment in order to determine the optimum frequency to use as a sub-maximal response, normally 4 Hz. A ventral midline incision was made, at the caudal end of the pubis, to expose the pubic area. Connective tissue was removed to expose the tunica of the clitoris, ensuring that the wall was free from small blood vessels. The external vaginal wall was also exposed by removing any connective tissue. One laser Doppler flow probe was inserted 3 cm into the vagina, so that half the probe shaft was still visible. A second probe was positioned so that it lay just above the external clitoral wall. The position of these probes was then adjusted until a signal was obtained. A second probe was placed just above the surface of a blood vessel on the external vaginal wall. Both probes were clamped in position.

Data Recordal

Vaginal and clitoral blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue).

All data can be reported as mean±standard error of the mean (s.e.m.).

The compounds of the formula (I) can be administered alone but will generally be administered in admixture with a suitable pharmaceutical excipient, diluent or carrier selected with regard to the intended route of administration and standard pharmaceutical practice.

Accordingly the present invention provides for a composition comprising a compound of formula (I) and a pharmaceutically acceptable diluent or carrier.

For example, the compounds of the formula (I) can be administered orally, buccally or sublingually in the form of tablets, capsules, ovules, elixirs, solutions or suspensions, which may contain flavouring or colouring agents, for immediate-, delayed-, modified-, sustained-, pulsed- or controlled-release applications.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the formula (I) may be combined with various sweetening or flavouring agents, colouring matter or dyes, with emulsifying and/or suspending agents and with diluents such as water, ethanol, propylene glycol and glycerin, and combinations thereof.

The compounds of the formula (I) can also be administered parenterally, for example, intravenously, intra-arterially, intraperitoneally, intrathecally, intraventricularly, intraurethrally, intrasternally, intracranially, intramuscularly or subcutaneously, or they may be administered by infusion techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds of formula (I) can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the formula (I) and a suitable powder base such as lactose or starch.

Alternatively, the compounds of the formula (I) can be administered in the form of a suppository or pessary, or they may be applied topically in the form of a gel, hydrogel, lotion, solution, cream, ointment or dusting powder. The compounds of the formula (I) may also be dermally or transdermally administered, for example, by the use of a skin patch. They may also be administered by the pulmonary or rectal routes.

For application topically to the skin, the compounds of the formula (I) can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended

or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of the formula (I) may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

The invention is further illustrated by the following, non-limiting examples.

### EXAMPLE 1

3-(2,3-Dichloro-benzyl)-1-(4-hydroxy-benzyl)-1-(1-pyridin-2-yl-cyclohexylmethyl)-urea

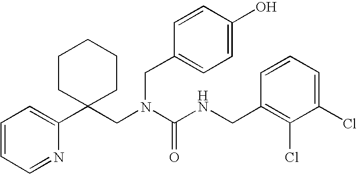

2,3-Dichlorobenzylamine (106 mg, 0.6 mmol) was dissolved in dichloromethane (2 ml) containing triethylamine (167 μl, 1.2 mmol) and was added over 5 minutes to a solution of triphosgene (57 mg, 0.2 mmol) in dichloromethane (5 ml) under a nitrogen atmosphere at 0° C. The mixture was stirred for 10 minutes and the amine from preparation 30 (148 mg, 0.5 mmol) was added and after stirring for 10 minutes, water (5 ml) was added. The phases were separated and the dichloromethane layer was evaporated under reduced pressure. The residue was purified by chromatography on a 10 g RediSep™ cartridge using ethyl acetate:heptanes (35:65) to give the title compound (142 mg).

¹H NMR (DMSO-d₆, 400 MHz): δ 1.17 (m, 2H), 1.24 (m, 1H), 1.53 (m, 5H), 2.38 (m, 2H), 3.37 (s, 2H), 3.56 (s, 2H), 4.17 (m, 2H), 6.68 (m, 4H), 6.83 (m, 1H), 6.96 (m, 1H), 7.23 (m, 2H), 7.48 (dd, 2H), 7.79 (dd, 1H), 8.60 (s, 1H), 9.23 (s, 1H).

LRMS (ES⁺) m/z 498, 500 [M+H]⁺

### EXAMPLES 2-5

The compounds of the following tabulated examples of the general formula:

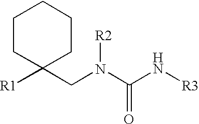

were prepared by a similar method to that of example 1 using the appropriate benzylamine and secondary amine.

| Ex. | | | |
|---|---|---|---|
| No | R1 | R2 | R3 |
| 2 | 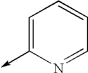 | 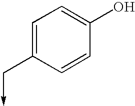 | 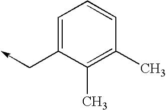 |
| 3 | 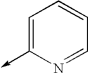 | 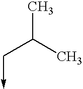 | 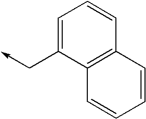 |
| 4 | 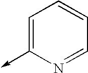 | 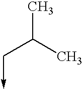 | 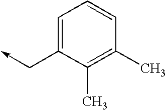 |
| 5 | 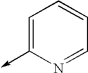 | 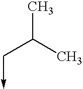 | 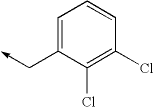 |

### EXAMPLE 2

¹H NMR (DMSO-d₆, 400 MHz): δ 1.10 (m, 2H), 1.24 (m, 1H), 1.51 (m, 5H), 2.08 (s, 3H), 2.21 (s, 3H), 2.38 (m, 2H), 3.34 (m, 2H), 3.57 (s, 2H), 4.19 (d, 2H), 6.43 (dd, 1H), 6.60 (m, 3H), 6.83 (d, 1H), 6.98 (dd, 2H), 7.22 (m, 1H), 7.41 (d, 2H), 7.78 (dd, 1H), 8.59 (s, 1H), 9.20 (s, 1H).

LRMS (ES⁺) m/z 458 [M+H]⁺

### EXAMPLE 3

¹H NMR (DMSO-d₆, 400 MHz): δ 0.58 (d, 6H), 1.10 (m, 2H), 1.20 (m, 1H), 1.50 (m, 5H), 1.70 (m, 1H), 2.28 (m, 4H), 3.43 (m, 2H), 4.62 (d, 2H), 6.60 (dd, 1H), 7.19 (m, 1H), 7.39 (m, 2H), 7.46 (dd, 1H), 7.51 (m, 2H), 7.71 (dd, 1H), 7.81 (d, 1H), 7.92 (m, 1H), 8.17 (m, 1H), 8.58 (m, 1H).

LRMS (ES⁺) m/z 430 [M+H]⁺

### EXAMPLE 4

¹H NMR (DMSO-d₆, 400 MHz): δ 0.58 (d, 6H), 1.10 (m, 2H), 1.22 (m, 1H), 1.51 (m, 5H), 1.41 (s, 1H), 2.12 (s, 3H), 2.29 (m, 7H), 3.44 (s, 2H), 4.18 (d, 2H), 6.48 (dd, 1H), 7.01 (m, 3H), 7.20 (m, 1H), 7.40 (d, 1H), 7.71 (dd, 1H), 8.57 (d, 1H),

LRMS (ES⁺) m/z 408 [M+H]⁺

### EXAMPLE 5

¹H NMR (DMSO-d₆, 400 MHz): δ 0.58 (d, 6H), 1.13 (m, 2H), 1.24 (m, 1H), 1.65 (m, 5H), 1.73 (m, 1H), 2.31 (m, 4H), 3.46 (s, 2H), 4.16 (d, 2H), 6.73 (dd, 1H), 7.11 (m, 2H), 7.17 (dd, 1H), 7.41 (d, 1H), 7.51 (d, 1H), 7.74 (d, 1H), 8.57 (d, 1H).

LRMS (ES⁺) m/z 448, 450 [M+H]⁺

### EXAMPLE 6

3-(2,6-Diisopropyl-phenyl]-1-(4-hydroxy-benzyl)-1-(1-methoxymethyl-cyclohexylmethyl)-urea

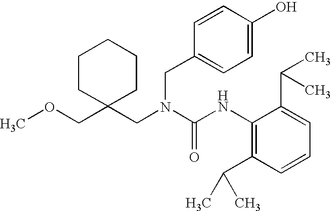

2,6-Diisopropylphenyl isocyanate (250 mg, 1.2 mmol) was added to a solution of the amine from preparation 20 (300 mg, 1.1 mmol) in dichloromethane (20 ml) and was stirred for 1 hour. The reaction mixture was evaporated under reduced pressure and the residue purified by chromatography on silica gel using 10-40% ethyl acetate in heptane to give the title compound (420 mg).

¹H NMR (CDCl₃, 400 MHz): δ 1.13 (d, 6H), 1.23 (m, 6H), 1.39 (m, 4H), 1.56 (m, 9H), 3.06 (m, 2H), 3.29 (s, 3H), 3.42 (m, 2H), 5.53 (s, 2H), 5.58 (s, 1H), 6.66 (d, 2H), 7.11 (m, 4H), 7.21 (d, 1H).

LRMS (ES⁺) m/z 467 [M+H]⁺

### EXAMPLES 7-10

The compounds of the following tabulated examples of the general formula:

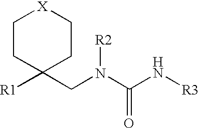

were prepared by a similar method to that of example 6 using the appropriate benzylamine and substituted isocyanate.

| Ex. | | | | |
|---|---|---|---|---|
| No | R1 | X | R2 | R3 |
| 7 | 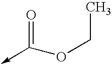 | -CH₂- | 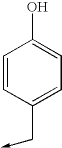 | 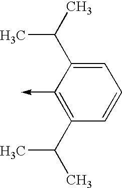 |
| 8 | 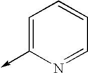 | -O- | 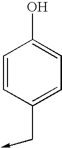 | 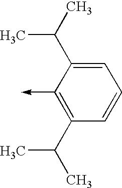 |
| 9 | 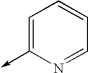 | -CH₂- | 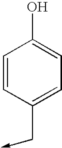 | 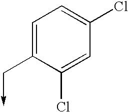 |
| 10 | 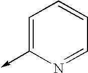 | -CH₂- | 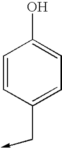 | 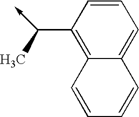 |

### EXAMPLE 7

¹H NMR (CDCl₃, 400 MHz): δ 1.15 (m, 20H), 1.60 (m, 3H), 2.09 (m, 2H), 2.81 (m, 2H), 3.72 (s, 2H), 4.19 (q, 2H), 4.45 (s, 2H), 5.06 (s, 1H), 5.72 (s, 1H), 6.79 (d, 2H), 7.07 (d, 2H), 7.19 (m, 3H).

LRMS (AP⁺) m/z 495 [M+H]⁺

### EXAMPLE 8

¹H NMR (CDCl₃, 400 MHz): δ 0.96 (s, 6H), 1.14 (s, 6H), 2.13 (m, 2H), 2.38 (d, 2H), 2.71 (m, 2H), 3.53 (t, 2H), 3.81, (s, 2H), 3.91 (m, 4H), 5.79 (s, 1H), 5.92 (s, 1H), 6.72 (d, 2H), 6.98 (d, 2H), 7.06 (d, 2H), 7.19 (m, 2H), 7.42 (d, 1H), 7.73 (dd, 1H), 8.64 (d, 1H).

LRMS (AP⁺) m/z 502 [M+H]⁺

### EXAMPLE 9

¹H NMR (CDCl₃, 400 MHz): δ 1.26 (m, 4H), 1.59 (m, 4H), 2.44 (d, 2H), 3.50 (s, 2H), 3.97 (s, 2H), 4.24 (d, 2H), 4.93 (t, 1H), 6.53 (s, 1H), 6.59 (d, 2H), 6.79 (d, 2H), 6.99 (d, 1H), 7.09 (m, 2H), 7.29 (s, 1H), 7.39 (d, 1H), 7.63 (dd, 1H), 8.52 (d, 1H).

LRMS (AP⁺) m/z 497,499 [M+H]⁺

### EXAMPLE 10

¹H NMR (CDCl₃, 400 MHz): δ 1.20 (m, 4H), 1.22 (d, 3H), 1.58 (m, 4H), 2.39 (t, 2H), 3.43 (s, 2H), 4.02 (q, 2H), 4.72 (d, 1H), 5.66 (1H, m), 6.19 (s, 1H), 6.59 (d, 2H), 6.82 (d, 3H), 7.20 (d, 1H), 7.24 (m, 1H), 7.38 (dd, 1H), 7.44 (m, 3H), 7.73 (d, 1H), 7.82 (d, 1H), 8.01 (d, 1H), 8.27 (d, 1H).

LCMS (AP⁺) m/z 494 [M+H]⁺

### EXAMPLE 11

3-(2,6-Diisopropyl-phenyl)-1-(4-hydroxy-benzyl)-1-(3-methyl-2-phenyl-butyl)-urea

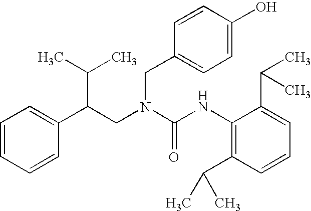

2,6-Diisopropylphenyl isocyanate (110 mg, 0.9 mmol) was added to a solution of the amine from preparation 36 (220 mg, 0.7 mmol) in dichloromethane (20 ml). The mixture was stirred for 20 minutes and then the solvent was evaporated under reduced pressure. The residue was purified by chromatography on silica gel first using 0-10% diethyl ether in dichloromethane as eluant and then on a second column using 20-30% ethyl acetate in heptane to give the title compound (70 mg).

¹H NMR (CDCl₃, 400 MHz): δ 0.76 (d, 3H), 0.87 (s, 3H), 1.07, (m, 9H), 1.23 (s, 3H), 1.94 (m, 1H), 2.59 (s, 1H), 2.84 (m, 2H), 3.62 (dd, 1H), 3.97 (d, 1H), 4.04 (dd, 1H), 4.18 (d, 1H), 5.12 (s, 1H), 5.36 (s, 1H), 6.73 (d, 2H), 7.02 (d, 2H), 7.06 (s, 1H), 7.19 (m, 1H), 7.24 (m, 4H), 7.30 (m, 2H).

LRMS (ES⁺) m/z 473 [M+H]⁺

### EXAMPLE 12

3-(2,3-Dimethyl-benzyl)-1-isobutyl-1-[1-(5-methoxy-pyridin-2-yl)-cyclohexylmethyl]-urea

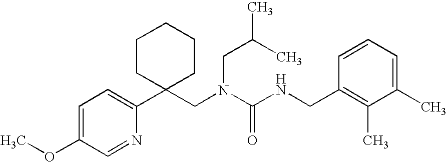

Sodium triacetoxyborohydride (422 mg, 2 mmol) was added portionwise to a solution of 2-methylpropionaldehyde (53 μl, 1 mmol) and [1-(5-methoxy-pyridin-2-yl)-cyclohexyl]-methylamine (WO 9807718) (220 mg, 1 mmol) in 1,2-dichloroethane (20 ml) and was stirred at room temperature for 18 hours. The reaction mixture was washed with sodium hydrogen carbonate solution (20 ml) and the isocyanate from preparation 32 (161 μl, 1 mmol) was added. The mixture was stirred for 10 minutes and then purified by chromatography on a 10 g RediSep™ cartridge using an elution gradient of ethyl acetate:heptane (10:90 to 50:50) then further purified by HPLC using 60-10% acetonitrile in water as eluant to give the title compound (115 mg).

¹H NMR (CDCl₃, 400 MHz): δ 0.79 (d, 6H), 1.22 (m, 3H), 1.56 (m, 5H), 1.96 (m, 1H), 2.14 (s, 3H), 2.28 (s, 3H), 2.38 (d, 2H), 2.76 (s, 2H), 3.39 (s, 2H), 3.80 (s, 3H), 4.14 (d, 2H), 4.30 (t, 1H), 6.98 (d, 1H), 7.10 (m, 3H), 7.22 (d, 1H), 8.07 (d, 1H).

LRMS (ES⁺) m/z 438 [M+H]⁺

### EXAMPLE 13

1-Benzyl-3-(2,6-diisopropyl-phenyl)-1-(4-phenyl-tetrahydro-pyran-4-ylmethyl)-urea

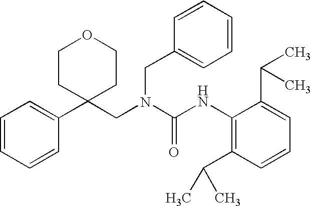

A solution of benzaldehyde (106 mg, 1 mmol) and the amine from preparation 34 (191 mg, 1 mmol) in toluene (50 ml) was heated at reflux using a Dean and Stark trap for 2 hours after which the solvent was evaporated under reduced pressure. The residue was dissolved in ethanol (15 ml), sodium borohydride (58 mg, 1.5 mmol) was added and the mixture was stirred for 18 hours at room temperature. The reaction mixture was diluted with water and the solvent evaporated under reduced pressure. The residue was partitioned between dichloromethane and 1 N sodium hydroxide solution. The aqueous solution was extracted twice with dichloromethane and the combined dichloromethane layers were dried over magnesium sulphate and evaporated under reduced pressure. The residue was dissolved in N,N-dimethylformamide (5 ml) and 2,6-diisopropylphenyl isocyanate (203 mg, 1 mmol) in N,N-dimethylformamide (2 ml) was added. The mixture was stirred at room temperature for 18 hours after which the solvent was evaporated under reduced pressure. The residue was purified by chromatography on silica gel using an elution gradient of ethyl acetate:heptane (0:100 to 20:80) and then on C18 silica using 0-10% methanol in water as eluant. The residue was recrystallised from heptane to give the title compound (213 mg).

¹H NMR (CDCl₃, 400 MHz): δ 0.92 (s, 6H), 1.19 (s, 6H), 2.08 (m, 4H), 2.66 (m, 2H), 3.60 (m, 2H), 3.73 (s, 2H), 3.81 (s, 2H), 3.90 (m, 2H), 5.39 (s, 1H), 7.10 (m, 4H), 7.19 (m, 1H), 7.30 (m, 4H) 7.41 (m, 4H). LCMS (AP⁺) m/z 500 [M+H]⁺

### EXAMPLE 14

1-Benzyl-3-(2,6-diisopropyl-phenyl)-1-(1-methyl-4-phenyl-piperidin-4-ylmethyl)-urea

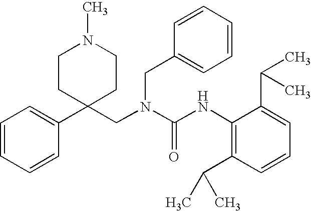

(1-Methyl-4-phenyl-piperidin-4-yl)-methylamine (Gazz. Chim. Ital. 86; 1956; 515) (204 mg, 1 mmol) and benzaldehyde (106 mg, 1 mmol) were dissolved in toluene (50 ml) and were heated at reflux using a Dean and Stark trap for 2 hours after which the solvent was evaporated under reduced pressure. The residue was dissolved in ethanol (15 ml) and sodium borohydride (57 mg, 1.5 mmol) was added. The mixture was stirred at room temperature for 18 hours and then water was added. The solvent was evaporated under reduced pressure and the residue was partitioned between dichloromethane and 1N sodium hydroxide solution. The aqueous solution was extracted twice with dichloromethane and the combined dichloromethane layers were dried over magnesium sulphate and evaporated under reduced pressure. The residue was dissolved in N,N-dimethylformamide (5 ml) and 2,6-diisopropylphenyl isocyanate (203 mg, 1 mmol) in N,N-dimethylformamide (2 ml) was added. The mixture was stirred at room temperature for 18 hours after which the solvent was evaporated under reduced pressure. The residue was dissolved in ethyl acetate and was washed with sodium carbonate solution (10% weight/volume), brine and then dried over magnesium sulphate. The solvent was evaporated under reduced pressure and the residue was purified by chromatography on C18 silica gel using 0-100% methanol in water as eluant to give the title compound (259 mg).

¹H NMR (CDCl₃, 400 MHz): δ 0.94 (s, 6H), 1.17 (s, 6H), 2.21 (m, 9H), 2.73 (m, 4H), 3.73 (s, 2H), 3.79 (m, 2H), 5.36 (s, 1H), 7.04 (m, 4H), 7.19 (m, 1H), 7.29 (m, 4H) 7.40 (m, 4H).

Found; C, 79.62; H, 8.80; N, 8.48; C₃₃H₄₃N₃O; requires C, 79.64; H, 8.71; N, 8.44%.

### EXAMPLE 15

3-(2,6-Diisopropyl-phenyl)-1-[2-methyl-2-(1-methyl-1H-indol-3-yl)-propyl]-1-(1-pyridin-2-yl-cyclohexylmethyl)-urea

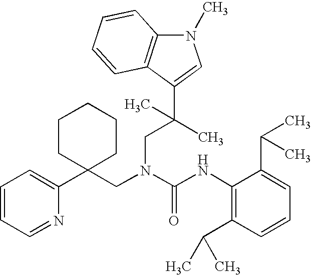

The amine from preparation 24 (114 mg, 0.6 mmol) and 2-methyl-2-(1-methyl-1H-indol-3-yl)-propionaldehyde (WO 9633211) (121 mg, 0.6 mmol) were dissolved in toluene and were heated at reflux using a Dean and Stark trap for 3.5 hours after which the solvent was evaporated under reduced pressure. The residue was dissolved in ethanol (10 ml) and sodium borohydride (35 mg, 0.9 mmol) was added. The mixture was stirred at room temperature for 18 hours and then water (500 μl) was added. The solvent was evaporated under reduced pressure and the residue was partitioned between dichloromethane and water. The aqueous solution was extracted twice with dichloromethane and the combined dichloromethane layers were dried over magnesium sulphate and evaporated under reduced pressure. The residue was dissolved in N,N-dimethylformamide (5 ml) and 2,6-diisopropylphenyl isocyanate (203 mg, 1 mmol) in N,N-dimethylformamide (2 ml) was added. The mixture was stirred at room temperature for 18 hours after which the solvent was evaporated under reduced pressure. The residue was purified by chromatography on silica gel using an elution gradient of ethyl acetate:heptane (0:100 to 20:80). The residue was recrystallised from ethyl acetate/heptane to give the title compound (148 mg).

¹H NMR (DMSO-d₆, 400 MHz): δ 1.05 (m, 2H), 1.14 (s, 6H), 1.15 (s, 6H), 1.46 (m, 6H), 1.30 (s, 6H), 2.24 (m, 2H), 3.23 (m, 6H), 3.66 (s, 3H), 6.82 (s, 1H), 7.10 (m, 8H), 7.32 (m, 1H), 7.50 (m, 1H), 7.60 (m, 1H), 8.49 (m, 1H).

### EXAMPLE 16

3-[3-(2,6-Diisopropyl-phenyl)-1-(1-pyridin-2-yl-cyclohexylmethyl)-ureidomethyl]-N-ethyl-benzamide.

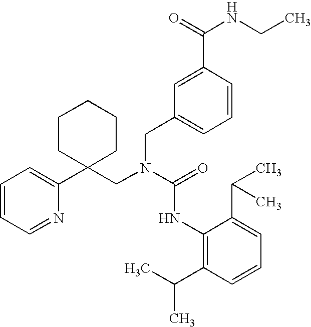

The secondary amine from preparation 37 (0.35 g, 1.0 mmol) was dissolved in chloroform (10 ml) and 2,6-diisopropylphenylisocyanate (0.22 ml, 1.02 mmol) was added, the mixture was stirred at room temperature for 48 hours. The solvent was evaporated under reduced pressure and the residue purified by chromatography on silica gel using ethyl acetate:hexanes (50:50) as eluant, to give the title compound (0.464 g).

LRMS (APCl) m/z 555 [M+H]⁺

HPLC Beckman C₁₈ 4.6×250 mm, 50:50 (water/acetonitrile)+0.1% trifluoroacetic acid, 1.5 ml/min, 214 nM, 4.074 min.

### EXAMPLES 17-30

The compounds of the following tabulated examples of the general formula:

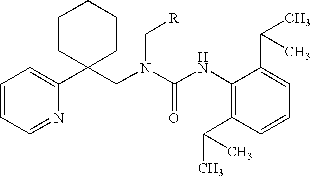

were prepared by a similar method to that of example 16 using the appropriate secondary amine and 2,6-diisopropylphenylisocyanate.

TABLE 3

| Example | | Yield | MS | |
|---|---|---|---|---|
| number | R | (%) | LRMS (APCI) m/z | Physical data |
| 17 | 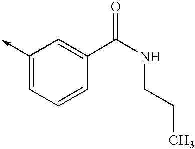 | 71 | 569 [M + H]⁺ | HPLC Beckman C₁₈ 4.6 × 250 mm, 50:50 (water/ acetonitrile) + 0.1% trifluoroacetic acid, 1.5 ml/min, 214 nm, 5.171 min. |
| 18 | 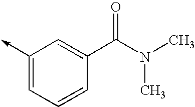 | 90 | 555 [M + H]⁺ | mp 187-188° C. |
| 19 | 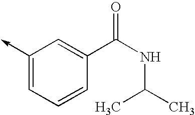 | 90 | 569 [M + H]⁺ | mp 190-191° C. |
| 20 (a) | 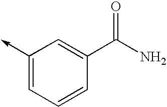 | 76 | 527 [M + H]⁺ | HPLC Beckman C₁₈ 4.6 × 250 mm, 50:50 (water/ acetonitrile) + 0.1% trifluoroacetic acid, 1.5 ml/min, 214 nm, 3.084 min. |
| 21 (a) | 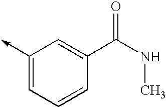 | 67 | 541 [M + H]⁺ | mp 155-156° C. |
| 22 (b) | 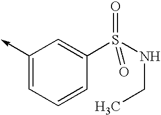 | 50 | 591 [M + H]⁺ | mp 149.1-149.5° C. |
| 23 (b) | 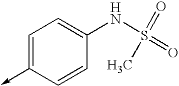 | 38 | 577 [M + H]⁺ | |
| 24 (b) | 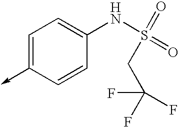 | 78 | 645 [M + H]⁺ | mp 197-198° (dec). |
| 25 | 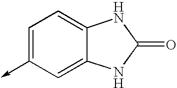 | 55 | 540 [M + H]⁺ | mp 159-161° |
| 26 (c) | 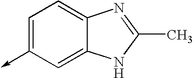 | 63 | 538 [M + H]⁺ | mp 154-156° |
| 27 (c) | 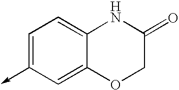 | 86 | 555 [M + H]⁺ | mp 115-118° |
| 28 (c) | 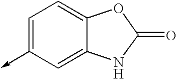 | 91 | 541 [M + H]⁺ | mp 176-117° |
| 29 | 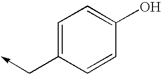 | 70 | 514 [M + H]⁺ | HPLC Beckman C₁₈ 4.6 × 250 mm, 50:50 (water/ acetonitrile) + 0.1% trifluoroacetic acid, 1.5 ml/min, 214 nm, 5.515 min. |
| 30 (b) | 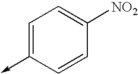 | 91 | | m.p. 144.3-145.5° C. |

(a)-Ethyl acetate was used as the reaction solvent, and the product was isolated by filtration from the reaction mixture.
(b)-dichloromethane was used as the reaction solvent. The product was isolated without chromatography after trituration with diethyl ether.
(c)-tetrahydrofuran was used as the reaction solvent, and the product was isolated without chromatography.

### EXAMPLE 31

1-(4-Amino-benzyl)-3-(2,6-diisopropyl-phenyl)-1-(1-pyridin-2-yl-cyclohexylmethyl)-urea

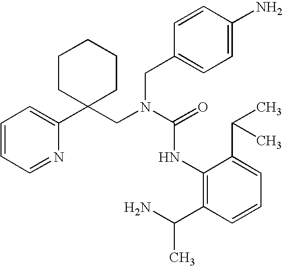

The nitro compound from example 30 (7.28 g, 13.77 mmol) and Raney® Nickel (5.4 g) in methanol (50 ml) and tetrahydrofuran (50 ml) was hydrogenated at 50 psi for 10 hours. The reaction mixture was filtered and the filtrate concentrated to give the title compound.

LRMS (APCl) m/z 499 [M+H]⁺

### EXAMPLE 32

Ethanesulphonic acid {4-[3-(2,6-diisopropyl-phenyl)-1-(1-pyridin-2-yl-cyclohexylmethyl)-ureidomethyl]-phenyl}-amide

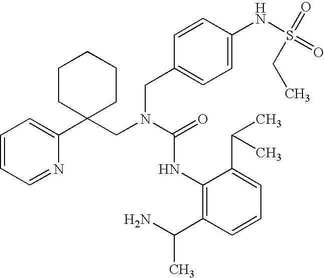

Ethane sulphonyl chloride (0.3 ml, 3.2 mmol) was added to the aniline from example 31 (500 mg, 1.0 mmol), and triethylamine (1 ml) in dichloromethane (50 ml) and the reaction stirred at room temperature for 18 hours. The mixture was partially purified by medium pressure liquid chromatography eluting with ethyl acetate:hexanes (33.3:66.6). The impure fractions were concentrated and diluted with methanol containing 10% by volume 1N lithium hydroxide solution. After 18 hours the mixture was further purified by medium pressure liquid chromatography eluting with ethyl acetate:hexanes (33.3: 66.6) to give a further quantity of the title compound. The two crops of title compound were combined and triturated with hot diethyl ether to provided the desired compound (177 mg).

mp 208.7-209.2° C.;

LRMS (APCl) m/z 591.3 [M+H]⁺

### EXAMPLE 33

1-Benzyl-3-(2-isopropenyl-6-isopropyl-phenyl)-1-(1-pyridin-2-yl-cyclohexylmethyl)-urea

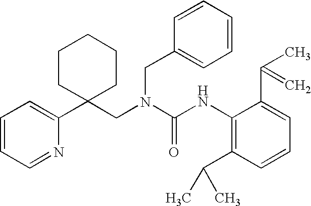

The aniline from preparation 58 (175 mg, 1 mmol) was dissolved in dichloromethane (2 ml) containing pyridine (162 μl, 2 mmol) and was added over 30 minutes to a solution of triphosgene (99 mg, 0.33 mmol) in dichloromethane (30 ml) under a nitrogen atmosphere at 0° C. The mixture was stirred for 30 minutes, the amine from preparation 25 (1.4 g, 5 mmol) was added and stirred for 30 minutes. The reaction mixture was washed with water, the dichloromethane layer was dried over magnesium sulphate and was purified by chromatography on a 40 g Biotage® SPE cartridge using ethyl acetate:heptane (0:100 to 100:0) to give the title compound (150 mg).

¹H NMR (CDCl₃, 400 MHz): δ 1.06 (d, 6H), 1.30 (m, 3H), 1.37 (m, 4H), 1.53 (m, 1H), 1.71 (m, 4H), 1.93 (s, 3H), 2.47 (m, 2H), 2.80 (m, 1H), 3.68 (s, 2H), 4.78 (s, 1H), 5.00 (s, 1H), 5.82 (s, 1H), 6.93 (m, 1H), 7.08 (m, 6H), 7.43 (d, 1H), 7.67 (dd, 1H), 8.62 (s, 1H).

LRMS (AP⁺) m/z 482 [M+H]⁺

### EXAMPLE 34

3-(2-Acetyl-6-isopropyl-phenyl)-1-benzyl-1-(1-pyridin-2-yl-cyclohexylmethyl)-urea

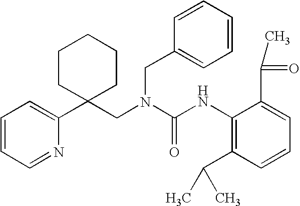

Oxygen was bubbled through a solution of the alkene from example 33 (1 g, 2.07 mmol) in dichloromethane (100 ml) for 10 minutes, to flush the system. The solution was then cooled in a dry ice/acetone bath, and ozone bubbled through for 15 minutes. Oxygen was bubbled through again for 5 minutes, followed by nitrogen for 15 minutes to purge the system. Methanol (2 ml) was added, followed by triphenylphosphine (545 mg), and the mixture allowed to warm slowly to room temperature, under a nitrogen atmosphere. The mixture was then concentrated under reduced pressure and the residue purified by column chromatography using a 25 g IST™ SPE cartridge using an elution gradient of ethyl acetate:heptane (0:100 to 20:80). The product was recrystallised from diethyl ether:heptane to afford the title compound, 880 mg.

¹H NMR (CDCl₃, 400 MHz): δ 0.98 (d, 6H), 1.28 (m, 3H), 1.65 (m, 5H), 2.47 (d, 2H), 2.59 (s, 3H), 3.69 (s, 1H), 3.71 (s, 2H), 5.74 (s, 2H), 7.03 (d, 2H), 7.19 (m, 5H), 7.39 (d, 1H), 7.51 (m, 2H), 7.73 (dd, 1H), 8.19 (s, 1H), 8.65 (d, 1H).

LRMS (AP⁺) m/z 484 [M+H]⁺

### EXAMPLE 35

1-Benzyl-3-[2-(1-hydroxy-1-methyl-ethyl)-6-isopropyl-phenyl-1-(1-pyridin-2-yl-cyclohexylmethyl)-urea

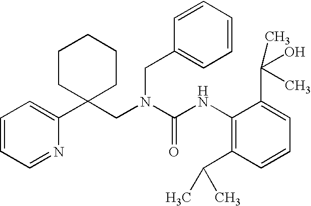

Methyl magnesium chloride (3M solution in tetrahydrofuran, 0.33 ml, 1 mmol) was added to a solution of the ketone from example 34 (242 mg, 0.5 mmol) in tetrahydrofuran (5 ml) at 0° C. The mixture was stirred for an hour and further methyl magnesium chloride (3M solution in tetrahydrofuran, 0.33 ml, 1 mmol) was added. The mixture was heated at reflux for 2.5 hours and then stirred at room temperature for 18 hours. The mixture was purified by chromatography on a 40 g Biotage® cartridge using an elution gradient of ethyl acetate:heptane (10:90 to 25:75). The fractions containing the title compound were further purified by chromatography on reverse phase silica, to afford the title compound (19 mg).

¹H NMR (CDCl₃, 400 MHz): δ 0.84 (s, 3H), 1.32 (m, 6H), 1.64 (m, 12H), 2.50 (d, 2H), 3.01 (m, 1H), 3.49 (m, 2H), 4.06 (m, 2H), 7.07 (m, 3H), 7.20 (m, 6H), 7.43 (d, 1H), 7.71 (dd, 1H), 7.85 (s, 1H), 8.63 (s, 1H).

LRMS (AP⁺) m/z 500 [M+H]⁺

### EXAMPLE 36

Benzyl-3-[2-(1-hydroxy-ethyl)-6-isopropyl-phenyl]-1-(1-pyridin-2-yl-cyclohexylmethyl)-urea

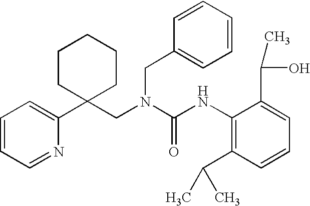

Sodium borohydride (57 mg, 1.5 mmol) was added to a stirred solution of the ketone from example 34 (483.7 mg, 1 mmol) in ethanol (20 ml). The mixture was stirred for 30 minutes and then water was added. The ethanol was evaporated under reduced pressure and the residue was partitioned between sodium bicarbonate solution and ethyl acetate. The ethyl acetate layer was washed with sodium bicarbonate solution, dried over magnesium sulphate and evaporated under reduced pressure to give the title compound (480 mg).

¹H NMR (CDCl₃, 400 MHz): δ 1.06 (d, 6H), 1.40 (m, 6H), 1.70 (m, 5H), 2.44 (d, 2H), 2.54 (d, 1H), 2.69 (m, 1H), 3.77 (m, 2H), 3.94 (m, 1H), 4.10 (s, 1H), 4.57 (s, 1H), 6.04 (s, 1H), 7.16 (m, 4H), 7.30 (m, 5H), 7.51 (d, 1H), 7.74 (dd, 1H), 8.59 (s, 1H).

LRMS (ES⁺) m/z 486 [M+H]⁺

### EXAMPLE 37

3-(2,6-Bis-dimethylamino-phenyl)-1-(4-hydroxy-benzyl)-1-(1-pyridin-2-yl-cyclohexylmethyl)-urea

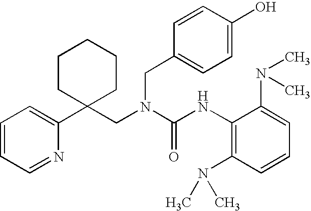

The N¹,N¹,N³,N³-tetramethyl-1,2,3-benzenetriamine (J. Med. Chem. 1993; 36 (22); 3300) (179 mg, 1 mmol) was dissolved in dichloromethane (5 ml) containing triethylamine (139 μl, 2 mmol) and was added to a solution of triphosgene (119 mg, 0.4 mmol) in dichloromethane (10 ml) at 0° C. The mixture was stirred for 5 minutes, the amine from preparation 34 (280 mg, 14 mmol) was added and stirred for 75 hours. The mixture was heated at reflux for 5 hours and then stirred at room temperature for 18 hours. The solvent was evaporated under reduced pressure and the residue was diluted with diethyl ether. The solid obtained was removed by filtration and the filtrate was concentrated under reduced pressure. The residue was purified by chromatography on a 20 g SPE cartridge using an elution gradient of ethyl acetate:heptane (0:100 to 40:60) as eluant to give the title compound (50 mg). Mp 162-166° C.

¹H NMR (CDCl₃, 400 MHz): δ 1.27 (m, 4H), 1.54 (m, 1H), 1.78 (m, 3H), 2.51 (m, 14H), 3.68 (s, 2H), 3.80 (s, 2H), 6.40 (s, 1H), 6.62 (d, 2H), 6.79 (d, 2H), 6.88 (m, 2H), 7.04 (dd, 1H), 7.19 (dd, 1H), 7.46 (d, 1H), 7.71 (dd, 1H), 8.64 (s, 1H).

LRMS (AP⁺) m/z 502 [M+H]⁺

### EXAMPLE 38

3-(2,6-Diisopropyl-phenyl)-1-(4-hydroxy-benzyl)-1-[1-(2-hydroxy-phenyl)-cyclohexylmethyl]-urea

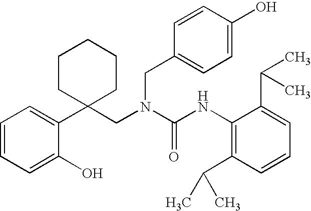

2,6-Diisopropylphenylisocyanate (150 mg, 0.74 mmol) was added to a solution of the amine from preparation 55 (140 mg, 0.45 mmol) in dichloromethane (20 ml), and the reaction stirred at room temperature for 18 hours. The mixture was concentrate under reduced pressure and the residue purified by column chromatography on silica gel using an elution gradient of dichloromethane:diethyl ether (100:0 to 90:10). The product was recrystallised from diethyl ether:heptane to afford the title compound as a white solid, 100 mg.

Mp 171-173° C.

¹H NMR (CDCl₃, 400 MHz): δ 0.93 (m, 7H), 1.13 (m, 6H), 1.39 (m, 2H), 1.62 (m, 3H), 1.78 (t, 2H), 2.61 (s, 4H), 3.84 (s, 2H), 4.47 (s, 2H), 4.81 (s, 1H), 5.64 (s, 1H), 6.80 (d, 2H), 6.82 (d, 2H), 6.96 (dd, 1H), 7.14 (m, 7H)

LRMS (AP⁺) m/z 515 [M+H]⁺

### EXAMPLE 39

3-(2,6-Diisopropyl-phenyl)-1-(4-hydroxy-benzyl)-1-(1-pyridin-2-yl-cyclohexylmethyl)-urea

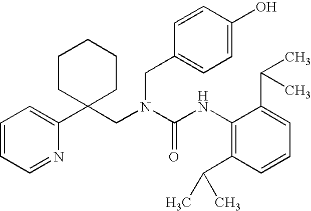

A solution of 4-hydroxybenzaldehyde (6.1 g, 50 mmol) and the amine from preparation 24 (9.5 g, 50 mmol) in toluene (75 ml) was refluxed in Dean-Stark apparatus for 2 hours. The toluene was removed and the residue taken up in abs. ethanol (80 ml), cooled to 0° C. and sodium borohydride (1.89 g, 50 mmol) added portionwise over 1 hour. The mixture was stirred for a further 3 hours (0-10° C.). The reaction mixture was diluted with ethyl acetate, washed with saturated sodium bicarbonate solution and dried (MgSO₄), removing solvent under reduced pressure at 30° C. The product was purified by chromatography (80 g Biotage® column, 10-30% ethyl acetate in heptane). This material was then taken up in ether and allowed to crystallise in the freezer over the weekend. Filtration and subsequent crops from concentrated filtrates gave a total yield of 10.5 g.

2,6-Diisopropylphenyl isocyanate (2.03 g, 10 mmol) was added to a solution of this secondary amine (2.96 g, 10 mmol) in dichloromethane (20 ml) and the reaction was stirred for 10 mins. The solvent was removed under reduced pressure and the residue was partially purified by chromatography (90 g Biotage SPE, 25% ethyl acetate in heptane). Crystallisation from methanol gave the title product, 3.54 g.

¹H NMR (DMSO-d₆, 400 MHz): δ 1.06 (m, 12H), 1.12-1.30 (m, 3H), 1.43-1.72 (m, 5H), 2.39 (m, 2H), 3.00 (m, 2H), 3.46 (s, 2H), 3.60 (s, 2H), 6.64 (d, 2H), 6.78 (d, 2H), 7.08 (d, 2H), 7.19 (dd, 1H), 7.28 (m, 1H), 7.46 (d, 1H), 7.54 (s, 1H), 7.81 (m, 1H), 8.63 (d, 1H).

LRMS (ES⁺) m/z 499 [M+H]⁺

### EXAMPLE 40

Morpholin-4-yl-acetic acid 4-[3-(2,6-diisopropyl-phenyl)-1-(1-pyridin-2-yl-cyclohexylmethyl)-ureidomethyl]-phenyl Ester

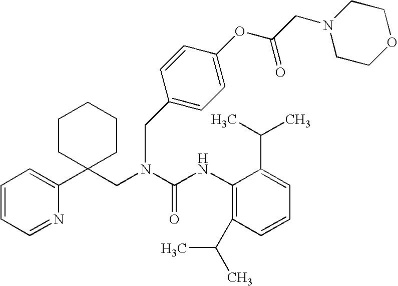

Morpholin-4-yl-acetic acid (J. Med. Chem. 36; 3;1993; 320) (116 mg, 0.8 mmol), was added to a solution of 1-(3-dimethylaminopropyl)-3-ethylcarbodiimide hydrochloride (230 mg, 1.2 mmol) and 4-(dimethylamino)pyridine (10 mg, 0.08 mmol) in dichloromethane (10 ml), and the mixture stirred for 10 minutes. The alcohol from example 39 (400 mg, 0.8 mmol) was added, and the reaction stirred at room temperature for 18 hours. The mixture was washed with water and brine, dried over magnesium sulphate and evaporated under reduced pressure. The crude product was purified by column chromatography on silica gel using an elution gradient of heptane:ethyl acetate (100:0 to 30:70) to afford the title compound, 280 mg.

¹H NMR (CDCl₃, 400 MHz) δ: 0.95-1.40 (m, 16H), 1.65-1.88 (m, 4H), 2.45-2.52 (m, 2H), 2.68 (m, 4H), 2.80 (m, 2H), 3.46 (s, 2H), 3.69 (s, 2H), 3.78 (m, 4H), 4.01 (s, 2H), 5.77 (s, 1H), 7.01 (d, 2H), 7.08 (d, 2H), 7.14-7.19 (m, 4H), 7.44 (d, 1H), 7.69 (m, 1H), 8.61 (m, 1H).

LRMS (ES⁺) m/z 627.47 [MH⁺]

### EXAMPLE 41

(4-Methyl-piperazin-4-yl)-acetic acid 4-[3-(2,6-diisopropyl-phenyl)-1-(1-pyridin-2-yl-cyclohexylmethyl)-ureidomethyl]-phenyl ester

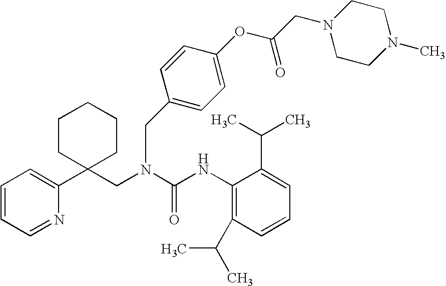

The title compound was obtained in 38% yield from the alcohol from example 39 and the acid from preparation 61, following a similar procedure to that described in example 42.

¹H NMR (CDCl₃, 400 MHz) δ: 0.99-1.39 (m, 16H), 1.61-1.78 (m, 4H), 2.30 (s, 3H), 2.47 (m, 5H), 2.70 (m, 4H), 2.80 (m, 2H), 3.44 (s, 2H), 3.68 (s, 2H), 4.00 (br s, 2H), 5.78 (br s, 1H), 7.00 (d, 2H), 7.07 (d, 2H), 7.18 (m, 4H), 7.42 (d, 1H), 7.68 (m, 1H), 8.60 (m, 1H).

LRMS (ES⁺) m/z 640.35 [M+H⁺]

### EXAMPLES 42 TO 51

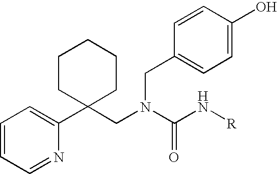

A solution of the amine from preparation 30 (0.5 ml in dichloromethane, 0.1 mmol) was added to an accurately known amount of the appropriate isocyanates (0.1 mmol). Acetonitrile was added to these mixtures to aid dissolution where necessary, and the reactions stood at room temperature for 18 hours. The reaction mixtures were purified by column chromatography using SPE (IST®) cartridges and ethyl acetate:hexane as eluants, to afford the desired compounds.

TABLE 1

| | | | MS | |
|---|---|---|---|---|
| Example | | Yield | (LRMS (ES⁺) | |
| number | R | (%) | m/z | ¹H NMR (DMSO-d₆, 400 MHz): δ |
| 42 | 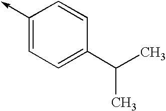 | 92 | 458 [M + H]⁺ | 1.10-1.30 (m, 9H), 1.42-1.60 (m, 5H), 2.38 (m, 2H), 2.80 (m, 1H), 3.42 (s, 2H), 3.65 (s, 2H), 6.62 (d, 2H), 6.72 (d, 2H), 7.06 (d, 2H), 7.26 (m, 3H), 7.45 (d, 1H), 7.78 (dd, 1H), 8.14 (s, 1H), 8.63 (s, 1H), 9.23 (s, 1H). |
| 43 | 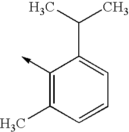 | 70 | | 1.01 (m, 6H), 1.19 (m, 3H), 1.58 (m, 5H), 2.08 (s, 3H), 2.40 (m, 2H), 2.94 (m, 1H), 3.44 (m, 2H), 3.55 (m, 2H), 6.64 (d, 2H), 6.75 (d, 2H), 7.00 (m, 1H), 7.08 (m, 2H), 7.27 (m, 1H), 7.47 (d, 1H), 7.53 (s, 1H), 7.81 (dd, 1H), 8.63 (d, 1H) 9.24 (s, 1H). |
| 44 | 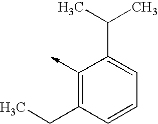 | 70 | 486 [M + H]⁺ | 1.02 (m, 9H), 1.10-1.30 (m, 3H), 1.44-1.60 (m, 3H), 1.63 (m, 2H), 2.40 (m, 4H), 2.98 (m, 1H), 3.44 (m, 2H), 3.60 (s, 2H), 6.63 (d, 2H), 6.76 (d, 2H), 7.01 (d, 1H), 7.09 (d, 1H), 7.15 (dd, 1H), 7.29 (m, 1H), 7.45 (d, 1H), 7.54 (s, 1H), 7.80 (m, 1H), 8.63 (d, 1H), 9.22 (s, 1H) |
| 45 | 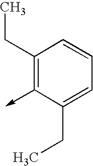 | 94 | 475 [M + H]⁺ | 1.03 (m, 6H), 1.19 (m, 3H), 1.58 (m, 5H), 2.41 (m, 6H), 3.44 (m, 2H), 3.60 (m, 2H), 6.64 (d, 2H), 6.75 (d, 2H), 7.02 (m, 1H), 7.10 (m, 2H), 7.27 (m, 1H), 7.46 (d, 1H), 7.54 (s, 1H), 7.80 (dd, 1H), 8.63 (d, 1H) 9.24 (s, 1H). |
| 46 | 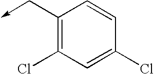 | 80 | 498, 501 [M + H]⁺ | 1.10 (m, 2H), 1.22 (m, 1H), 1.31 (m, 5H), 2.37 (m, 2H), 3.36 (s, 2H), 3.53 (s, 2H), 4.18 (s, 2H), 6.62 (m, 4H), 6.89 (m, 1H), 7.10 (d, 1H), 7.22 (m, 1H), 7.36 (s, 1H), 7.41 (d, 1H), 7.50 (d, 1H), 7.78 (m, 1H), 8.60 (d, 1H), 9.23 (s, 1H). |
| 48 | 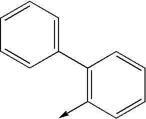 | 87 | 488, [M + H]⁺ | 1.10 (m, 3H), 1.18 (m, 2H), 1.35 (m, 1H), 1.48 (m, 2H), 2.24 (m, 2H), 3.21 (m, 2H), 3.38 (m, 2H), 6.58 (m, 4H), 7.32 (m, 12H), 7.77 (t, 1H), 8.58 (d, 1H) 9.24 (s, 1H). |
| 49 | 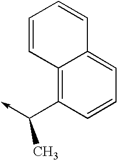 | 89 | 444 [M + H]⁺ | 1.10 (m, 2H), 1.20 (m, 1H), 1.50 (m, 8H), 2.30 (m, 2H), 3.30 (m, 2H), 3.60 (m, 2H), 5.60 (m, 1H), 6.49 (d, 1H), 6.58 (m, 2H), 6.64 (d, 2H), 7.21 (m, 1H), 7.39 (m, 3H), 7.53 (m, 2H), 7.78 (m, 2H), 7.92 (d, 1H), 8.15 (d, 1H), 8.58 (d, 1H) 9.21 (s, 1H). |
| 50 | 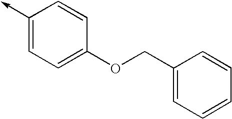 | 63 | 522 [M + H]⁺ | 1.14 (m, 2H), 1.24 (m, 1H), 1.42-1.60 (m, 5H), 2.39 (m, 2H), 3.42 (s, 2H), 3.64 (s, 2H), 5.02 (s, 2H), 6.62 (d, 2H), 6.73 (d, 2H), 6.86 (d, 2H), 7.23 (m, 3H), 7.30-7.50 (m, 6H), 7.78 (m, 1H), 8.06 (s, 1H), 8.62 (d, 1H), 9.23 (s, 1H). |
| 51 | 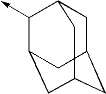 | 17 | 474 [M + H]⁺ | 1.12 (m, 2H), 1.23 (m, 1H), 1.38 (m, 2H), 1.41-1.80 (m, 16H), 2.38 (m, 2H), 3.40 (s, 2H), 3.59 (m, 1H), 3.72 (s, 2H), 5.17 (d, 1H), 6.15 (d, 1H), 6.62 (d, 2H), 6.78 (d, 2H), 7.22 (m, 1H), 7.42 (d, 1H), 7.77 (m, 1H), 8.60 (d, 1H), 9.23 (s, 1H). |

### EXAMPLE 52

1-(2,3-Dihydroxy-propyl)-3-(2,6-diisopropyl-phenyl)-1-(1-pyridin-2-yl-cyclohexylmethyl)-urea

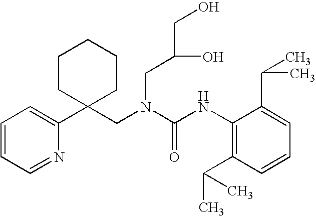

The aldehyde from preparation 28 (37.9 mg, 0.2 mmol) in 1,2-dichloroethane (3 ml) was added to (+/−)-3-amino-1,2-propanediol (22.2 mg, 0.2 mmol) in 1,2-dichloroethane (3 ml), then sodium triacetoxyborohydride (84.8 mg, 0.4 mmol) was added and the mixture was stirred for 18 hours at room temperature. Sodium hydrogen carbonate was added and the mixture was diluted with a saturated solution of sodium hydrogen carbonate (3 ml). The phases were separated using a Whatman® 12 ml PTFE membrane cartridge and the aqueous layer was washed with dichloromethane (3×2 ml). The organic phases were combined and 2,6-diisopropylphenylisocyanate (40.7 mg, 0.2 mmol) in 1,2-dichloroethane (1 ml) was added. After 18 hours the solvent was evaporated under a stream of nitrogen. The residue was purified by chromatography on a 10 g RediSep™ cartridge using ethyl acetate/heptanes to give the title compound (66 mg).

¹H NMR (400 MHz, DMSO-d₆): δ 1.13 (m, 15H), 1.51 (m, 5H), 2.38 (m, 2H), 2.64 (m, 2H), 3.09 (m, 4H), 3.50 (m, 2H), 3.61 (m, 1H), 4.15 (m, 1H), 4.48 (s, 1H), 7.09 (d, 2H), 7.19 (m, 2H), 7.41 (d, 1H), 7.61 (s, 1H), 7.78 (dd, 1H), 8.59 (d, 1H).

LRMS (APCl) m/z 468 (M+H)⁺

### EXAMPLES 53 TO 66

The compounds of the following tabulated examples of the general formula:

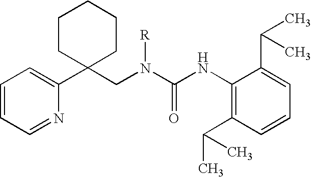

were prepared by a similar method to that of example 52 using the aldehyde from preparation 28 and the appropriate amine starting material.

| | | | MS data | |
|---|---|---|---|---|
| Example | | Yield | (LRMS (APCl) | |
| number | R | (%) | m/z) | ¹H NMR (DMSO-d₆, 400 MHz): δ |
| 53 | 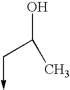 | 88 | | 0.79 (d, 3H), 1.11 (m, 15H), 1.56 (m, 5H), 2.33 (5, 2H), 2.41 (m, 1H), 2.58 (m, 1H), 3.12 (m, 2H), 3.50 (m, 1H), 3.63 (m, 1H), 3.70 (d, 1H), 4.48 (s, 1H), 7.09 (d, 2H), 7.20 (m, 2H), 7.42 (d, 1H), 7.66 (s, 1H), 7.78 (dd, 1H), 8.60 (d, 1H). |
| 54 | 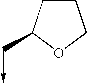 | 57 | 478 (M + H)⁺ | 1.13 (m, 16H), 1.60 (m, 8H), 2.32 (t, 2H), 2.43 (m, 1H), 2.64 (d, 1H), 3.12 (m, 2H), 3.43 (d, 1H), 3.59 (m, 2H), 3.77 (d, 2H), 7.07 (d, 2H), 7.20 (m, 2H), 7.42 (d, 1H), 7.57 (s, 1H), 7.79 (dd, 1H), 8.61 (d, 1H). |
| 55 | 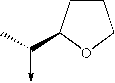 | 69 | 478 (M + H)⁺ | 1.13 (m, 16H), 1.60 (m, 8H), 2.32 (t, 2H), 2.43 (m, 1H), 2.64 (d, 1H), 3.12 (m, 2H), 3.43 (d, 1H), 3.59 (m, 2H), 3.77 (d, 2H), 7.20 (m, 2H), 7.42 (d, 1H), 7.57 (s, 1H), 7.79 (dd, 1H), 8.61 (d, 1H). |
| 56 | 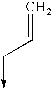 | 79 | 434 (M + H)⁺ | 1.14 (m, 15H), 1.60 (m, 5H), 2.36 (d, 2H), 3.10 (m, 4H), 3.42 (s, 2H), 4.87 (d, 1H), 5.04 (d, 1H), 5.56 (m, 1H), 7.07 (d, 2H), 7.21 (m, 2H), 7.43 (m, 2H), 7.79 (dd, 1H), 8.62 (d, 1H). |
| 57 | 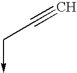 | 36 | 432 (M + H)⁺ | 1.11 (m, 15H), 1.61 (m, 5H), 2.34 (d, 2H), 3.14 (m, 4H), 3.35 (m, 1H), 3.50 (s, 1H), 7.11 (d, 2H), 7.22 (m, 2H), 7.43 (m, 2H), 7.70 (s, 1H), 7.79 (t, 1H), 8.62 (d, 1H). |
| 58 | 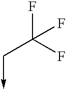 | 79 | 474 (M + H)⁺ | 1.12 (m, 15H), 1.60 (m, 5H), 2.42 (d, 2H), 3.13 (m, 4H), 3.64 (s, 2H), 7.13 (d, 2H), 7.23 (m, 1H), 7.30 (m, 1H), 7.50 (m, 1H), 7.70 (s, 1H), 7.84 (t, 1H), 7.98 (s, 1H), 8.66 (d, 1H). |
| 59 | 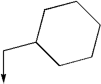 | 83 | 490 (M + H)⁺ | 0.63 (m, 2H), 1.11 (m, 19H), 1.54 (m, 11H), 2.30 (d, 3H), 3.14 (m, 2H), 3.52 (s, 2H), 7.10 (d, 2H), 7.19 (m, 1H), 7.23 (m, 1H), 7.44 (m, 2H), 7.70 (s, 1H), 7.84 (t, 1H), 8.61 (d, 1H). |
| 60 | 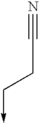 | 56 | 447 (M + H)⁺ | 1.13 (m, 15H), 1.60 (m, 5H), 2.42 (d, 2H), 2.76 (s, 2H), 3.08 (m, 2H), 3.17 (s, 2H), 3.58 (s, 2H), 7.11 (d, 2H), 7.20 (m, 1H), 7.27 (m, 1H), 7.47 (d, 1H), 7.72 (s, 1H), 7.82 (t, 1H), 7.98 (s, 1H), 8.64 (d, 1H). |
| 61 | 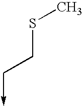 | 25 | 468 (M + H)⁺ | 1.11 (m, 15H), 1.59 (m, 5H), 1.88 (s, 3H), 2.39 (m, 4H), 2.59 (s, 2H), 3.10 (m, 2H), 3.53 (s, 2H), 7.10 (d, 2H), 7.20 (m, 1H), 7.28 (m, 1H), 7.62 (s, 1H), 7.81 (t, 1H), 8.63 (d, 1H). |
| 62 | 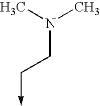 | 57 | 468 (M + H)⁺ | 1.13 (m, 15H), 1.60 (m, 5H), 2.04 (s. 6H), 2.10 (s, 2H), 2.30 (d, 2H), 2.64 (m, 2H), 3.08 (m, 2H), 3.55 (s, 2H), 7.08 (d, 2H), 7.18 (m, 1H), 7.24 (m, 1H), 7.45 (d, 1H), 7.81 (dd, 1H), 8.26 (s, 1H), 8.63 (d, 1H). |
| 63 | 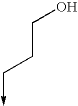 | 63 | 452 (M + H)⁺ | 1.12 (m, 16H), 1.53 (m, 7H), 2.34 (d, 2H), 2.60 (m, 2H), 3.09 (m, 2H), 3.20 (m, 2H), 3.46 (s, 2H), 7.11 (d, 2H), 7.21 (m, 2H), 7.43 (m, 2H), 7.78 (dd, 1H), 8.60 (d, 1H). |
| 64 | 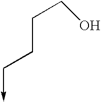 | 67 | 466 (M + H)⁺ | 1.12 (m, 16H), 1.50 (m, 9H), 2.33 (d, 2H), 2.45 (m, 2H), 3.13 (m, 2H), 3.26 (m, 2H), 3.47 (s, 2H), 7.08 (d, 2H), 7.20 (m, 2H), 7.41 (m, 1H), 7.47 (s, 1H), 7.78 (dd, 1H), 8.60 (d, 1H). |
| 65 | 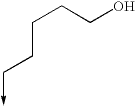 | 39 | 480 (M + H)⁺ | 1.12 (m, 16H), 1.50 (m, 11H), 2.30 (d, 2H), 2.41 (m, 2H), 3.13 (m, 2H), 3.29 (m, 2H), 3.47 (s, 2H), 7.08 (d, 2H), 7.20 (m, 2H), 7.42 (m, 1H), 7.46 (s, 1H), 7.79 (dd, 1H), 8.60 (d, 1H). |
| 66 | 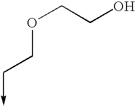 | 58 | 482 (M + H)⁺ | 1.12 (m, 16H), 1.47 (m, 5H), 2.36 (d, 2H), 2.68 (m, 2H), 3.10 (m, 2H), 3.32 (m, 4H), 3.43 (m, 2H), 3.53 (s, 2H), 7.09 (d, 2H), 7.23 (m, 2H), 7.43 (m, 1H), 7.53 (s, 1H), 7.79 (dd, 1H), 8.62 (d, 1H). |

### EXAMPLES 67 TO 126

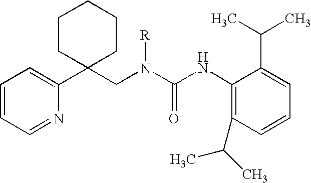

A solution of the amine from preparation 24 (1 molar equiv, 0.2M in 1,2-dichloroethane) was added to an accurately known amount of aldehyde (about 0.2 mol), and the total volume adjusted to 3 ml using 1,2-dichloroethane. Sodium triacetoxyborohydride (2 molar equiv) was added, and the reactions shaken for 18 hours. Sodium bicarbonate solution was added, the phases separated using Whatman® 6 ml PTFE membranes, and washed with dichloromethane (2 ml). 2,6-Diisopropylphenylisocyanate (1 molar equiv, 0.2M in 1,2-dichloroethane) was added to the organic phases and the reactions stirred for 10 minutes. The solvents were removed by heating at 60° C. for several hours. The residues were purified by column chromatography using an IST™ SPE silica cartridge using an elution gradient of ethyl acetate:heptane to afford the desired compounds.

| Ex | | Yield | MS | NMR |
|---|---|---|---|---|
| No | R | (%) | (ES⁺) | ¹H-NMR (400 MHz, DMSO-d₆): δ |
| 67 (a) | 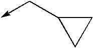 | 22 | 448 [M + H]⁺ | 0.06 (m, 2H), 0.38 (m, 2H), 0.90 (m, 12H), 1.14-1.38 (m, 3H), 1.54-1.77 (m, 5H), 2.40 (m, 3H), 3.20 (d, 2H), 3.64 (s, 2H), 7.18 (m, 2H), 7.26 (m, 2H), 7.46 (d, 1H), 7.59 (s, 1H), 7.82 (m, 1H), 8.64 (s, 1H). |
| 68 (a) | 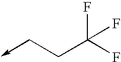 | 15 | 490 [M + H]⁺ | 1.02-1.34 (m, 15H), 1.44-1.71 (m, 5H), 2.27 (m, 2H), 2.40 (m, 2H), 2.60 (s, 2H), 3.05 (m, 2H), 3.42 (s, 2H), 7.12 (d, 2H), 7.22 (m, 2H), 7.45 (d, 1H), 7.70 (s, 1H), 7.80 (m, 1H), 7.80 (m, 1H), 8.61 (s, 1H). |
| 69 | 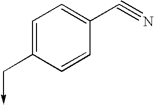 | 27 | 509 [M + H]⁺ | 1.00 (m, 12H), 1.14-1.68 (m, 8H), 2.39 (m, 2H), 2.86 (m, 2H), 3.60 (s, 2H), 3.84 (s, 2H), 7.05 (d, 2H), 7.16 (m, 3H), 7.27 (dd, 1H), 7.47 (d, 1H), 7.62 (s, 1H), 7.78 (m, 3H), 8.60 (d, 1H). |
| 70 | 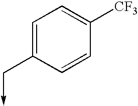 | 4 | 552 [M + H]⁺ | 1.00 (m, 12H), 1.13-1.34 (m, 3H), 1.44-1.76 (m, 5H), 2.39 (m, 2H), 2.84 (m, 2H), 3.61 (s, 2H), 3.86 (s, 2H), 7.03 (d, 2H), 7.19 (m, 3H), 7.28 (m, 1H), 7.49 (d, 1H), 7.60 (s, 1H), 7.64 (d, 2H), 7.82 (dd, 1H), 8.62 (d, 1H). |
| 71 (a) | 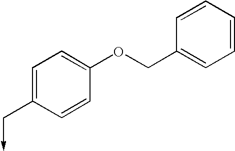 | 31 | 590 [M + H]⁺ | 1.02 (m, 12H), 1.10-1.30 (m, 3H), 1.43-1.70 (m, 5H), 2.39 (m, 2H), 2.98 (m, 2H), 3.48 (s, 2H), 3.64 (s, 2H), 6.89 (m, 4H), 7.08 (d, 2H), 7.19 (m, 1H), 7.25-7.50 (m, 8H), 7.57 (s, 1H), 7.80 (m, 1H), 8.62 (d, 1H). |
| 72 | 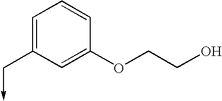 | 42 | 544 [M + H]⁺ | 0.96-1.36 (m, 15H), 1.54 (m, 3H), 1.63 (m, 2H), 2.39 (m, 2H), 2.96 (m, 2H), 3.52 (s, 2H), 3.69 (m, 4H), 3.92 (s, 2H), 4.82 (m, 1H), 6.50 (m, 2H), 6.78 (d, 1H), 7.06 (m, 2H), 7.18 (m, 2H), 7.29 (m, 1H), 7.48 (d, 1H), 7.58 (s, 1H), 7.80 (m, 1H), 8.62 (s, 1H). |
| 73 | 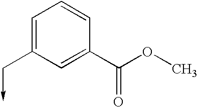 | 45 | 542 [M + H]⁺ | 0.99 (m, 12H), 1.17 (m, 3H), 1.42-1.60 (m, 3H), 1.64 (m, 2H), 2.38 (m, 2H), 2.92 (m, 2H), 3.58 (s, 2H), 3.82 (m, 5H), 7.05 (d, 2H), 7.19 (m, 2H), 7.26 (m, 1H), 7.45 (m, 2H), 7.61 (d, 2H), 7.81 (s, 2H), 8.62 (s, 1H). |
| 74 | 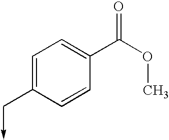 | 56 | 542 [M + H]⁺ | 1.00 (m,12H), 1.10-1.30 (m, 3H), 1.43-1.60 (m, 3H), 1.66 (m, 2H), 2.38 (m, 2H), 2.90 (m, 2H), 3.59 (s, 2H), 3.82 (m, 5H), 7.06 (m, 3H), 7.18 (m, 1H), 7.28 (m, 2H), 7.47 (d, 1H), 7.61 (s, 1H), 7.80 (dd, 1H), 7.86 (d, 2H), 8.61 (d, 1H). |
| 75 (a) | 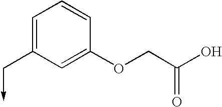 | 58 | 558 [M + H]⁺ | 1.03 (m, 12H), 1.12-1.26 (m, 3H), 1.44-1.58 (m, 3H), 1.66 (m, 2H), 2.38 (m, 2H), 2.99 (m, 2H), 3.50 (s, 2H), 3.62 (s, 2H), 4.02 (s, 2H), 6.37 (s, 1H), 6.45 (d, 1H), 6.60 (d, 1H), 7.09 (m, 3H), 7.18 (m, 1H), 7.27 (m, 1H), 7.47 (d, 1H), 7.58 (s, 1H), 7.82 (m, 1H), 8.64 (d, 1H). |
| 76 (c) | 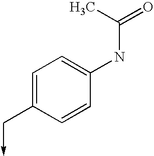 | 7 | 502 [M + H]⁺ | 1.02 (m, 12H), 1.12-1.28 (m, 3H), 1.44-1.72 (m, 5H), 2.00 (s, 3H), 2.39 (m, 2H), 2.98 (m, 2H), 3.50 (s, 2H), 3.66 (s, 2H), 6.85 (d, 2H), 7.07 (d, 2H), 7.19 (dd, 1H), 7.30 (m, 1H), 7.46 (m, 3H), 7.58 (s, 1H), 7.82 (m, 1H), 8.62 (d, 1H). |
| 77 (a) | 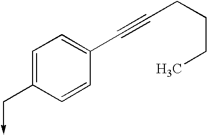 | 31 | 564 [M + H]⁺ | 0.92 (t, 3H), 1.02 (m, 12H), 1.09-1.30 (m, 3H), 1.38-1.72 (m, 9H), 2.34-2.42 (m, 4H), 2.94 (m, 2H), 3.54 (s, 2H), 3.75 (s, 2H), 6.92 (d, 2H), 7.06 (d, 2H), 7.18 (dd, 1H), 7.27 (m, 3H), 7.46 (d, 1H), 7.59 (s, 1H), 7.81 (dd, 1H), 8.61 (d, 1H). |
| 78 (a) | 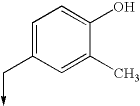 | 30 | 514 [M + H]⁺ | 1.01-1.30 (m, 15H), 1.43-1.70 (m, 5H), 2.06 (s, 3H), 2.38 (m, 2H), 3.00 (m, 2H), 3.43 (s, 2H), 3.58 (s, 2H), 6.55 (d, 1H), 6.65 (m, 2H), 7.08 (d, 2H), 7.19 (dd, 1H), 7.28 (m, 1H), 7.45 (d, 1H), 7.50 (s, 1H), 7.81 (m, 1H), 8.62 (d, 1H), 9.16 (br s, 1H). |
| 79 (a) | 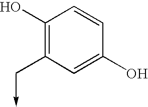 | 18 | 516 [M + H]⁺ | 0.98-1.30 (m, 15H), 1.45-1.74 (m, 5H), 2.41 (m, 2H), 2.99 (m, 2H), 3.54 (s, 2H), 3.65 (s, 2H), 6.38 (s, 1H), 6.42 (d, 1H), 6.50 (d, 1H), 7.05 (d, 2H), 7.18 (dd, 1H), 7.24 (m, 1H), 7.46 (m, 2H), 7.80 (dd, 1H), 8.58 (br s, 1H), 8.60 (s, 1H), 8.72 (br, s 1H). |
| 80 | 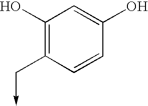 | 48 | 516 [M + H]⁺ | 1.01 (m, 12H), 1.12-1.30 (m, 3H), 1.50 (m, 3H), 1.63 (m, 2H), 2.39 (m, 2H), 2.98 (m, 2H0, 3.45 (s, 2H), 3.59 (s, 2H), 6.14 (d, 1H), 6.18 (s, 1H), 6.61 (d, 1H), 7.06 (d, 2H), 7.18 (m, 1H), 7.23 (m, 1H), 7.46 (m, 2H), 7.79 (m, 1H), 8.61 (d, 1H), 9.06 (s, 1H), 9.60 (s, 1H). |
| 81 (d) | 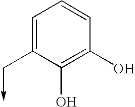 | 20 | 516 [M + H]⁺ | 0.99 (m, 12H), 1.15 (m, 3H), 1.52 (m, 3H), 1.65 (m, 2H), 2.41 (m, 2H), 2.98 (m, 2H), 3.50 (s, 2H), 3.75 (s, 2H), 6.35 (d, 1H), 6.57 (m, 1H), 6.62 (d, 1H), 7.04 (d, 2H), 7.17 (m, 1H), 7.22 (m, 1H), 7.50 (d, 2H), 7.80 (m, 1H), 8.60 (d, 1H), 9.03 (m, 1H). |
| 82 (d) | 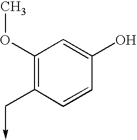 | 18 | 530 [M + H]⁺ | 1.00-1.29 (m, 15H), 1.42-1.70 (m, 5H), 2.37 (m, 2H), 3.02 (m, 2H), 3.40 (s, 2H), 3.60 (s, 3H), 3.68 (s, 2H), 6.26 (d, 1H), 6.34 (s, 1H), 6.70 (d, 1H), 7.06 (d, 2H), 7.18 (dd, 1H), 7.23 (m, 1H), 7.43 (m, 2H), 7.79 (m, 1H), 8.58 (d, 1H), 9.38 (br s, 1H). |
| 83 (d) | 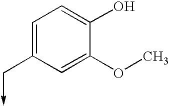 | 34 | 530 [M + H]⁺ | 1.04 (m, 12H), 1.15-1.72 (m, 8H), 2.40 (m, 2H), 2.98 (m, 2H), 3.52 (s, 2H), 3.62 (s, 2H), 3.70 (s, 3H), 6.37 (d, 1H), 6.48 (s, 1H), 6.64 (d, 1H), 7.07 (d, 2H), 7.19 (dd, 1H), 7.27 (m, 1H), 7.46 (m, 2H), 7.81 (m, 1H), 8.63 (d, 1H), 8.82 (br s, 1H). |
| 84 (a) | 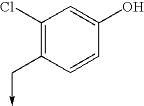 | 22 | 534, 536 [M + H]⁺ | 1.00-1.32 (m, 15H), 1.43-1.73 (m, 5H), 2.40 (m, 2H), 3.04 (m, 2H), 3.45 (s, 2H), 3.70 (s, 2H), 6.70 (d, 1H), 6.74 (s, 1H), 6.79 (d, 1H), 7.08 (d, 2H), 7.19 (dd, 1H), 7.25 (m, 1H), 7.45 (d, 1H), 7.72 (s, 1H), 7.80 (dd, 1H), 8.60 (d, 1H). |
| 85 (d) | 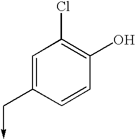 | 42 | 534, 536 [M + H]⁺ | 1.04 (m, 12H), 1.14-1.30 (m, 3H), 1.52 (m, 3H0, 1.64 (m, 2H), 2.38 (m, 2H), 2.97 (m, 2H), 3.50 (s, 2H), 3.60 (s, 2H), 6.66 (d, 1H), 6.84 (d, 1H), 6.94 (s, 1H), 7.06 (d, 2H), 7.19 (m, 1H), 7.28 (m, 1H), 7.46 (d, 1H), 7.58 (s, 1H), 7.81 (m, 1H), 8.62 (d, 1H), 10.00 (s, 1H). |
| 86 (a) | 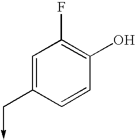 | 14 | 518 [M + H]⁺ | 0.98-1.35 (m, 15H), 1.54 (m, 3H), 1.63 (m, 2H), 2.39 (m, 2H), 2.97 (m, 2H), 3.50 (s, 2H), 3.60 (s, 2H), 6.57 (d, 1H), 6.70 (d, 1H), 6.83 (m, 1H), 7.06 (d, 2H), 7.19 (m, 1H), 7.28 (m, 1H), 7.45 (d, 1H), 7.58 (s, 1H), 7.80 (m, 1H), 8.62 (s, 1H), 9.64 (s, 1H). |
| 87 | 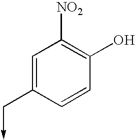 | 24 | 545 [M + H]⁺ | 1.02 (m, 12H), 1.10-1.30 (m, 3H), 1.44-1.75 (m, 5H), 2.40 (m, 2H), 2.90 (m, 2H), 3.58 (s, 2H), 3.74 (s, 2H), 7.01-7.15 (m, 4H), 7.19 (dd, 1H), 7.28 (dd, 1H), 7.48 (m, 2H), 7.60 (s, 1H), 7.80 (dd, 1H), 8.62 (d, 1H). |
| 88 | 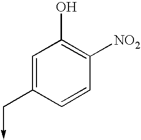 | 42 | 545 [M + H]⁺ | 0.99 (m, 12H), 1.12-1.34 (m, 3H), 1.52 (m, 3H), 1.64 (m, 2H), 2.38 (m, 2H), 2.88 (m, 2H), 3.60 (s, 2H), 3.80 (s, 2H), 6.59 (d, 1H), 6.77 (s, 1H), 7.04 (d, 2H), 7.18 (m, 1H), 7.26 (m, 1H), 7.46 (d, 1H), 7.59 (s, 1H), 7.81 (m, 2H), 8.61 (s, 1H), 10.84 (s, 1H). |
| 89 (a), (b) | 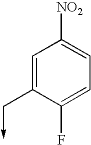 | 31 | 547 [M + H]⁺ | 1.00 (m, 12H), 1.14-132 (m, 3H), 1.45-1.76 (m, 5H), 2.42 (m, 2H), 2.97 (m, 2H), 3.64 (s, 2H), 3.85 (s, 2H), 7.07 (d, 2H), 7.19 (dd, 1H), 7.24 (m, 1H), 7.41 (dd, 1H), 7.48 (d, 1H), 7.79 (m, 2H), 7.91 (m, 1H), 8.20 (m, 1H), 8.60 (d, 1H). |
| 90 | 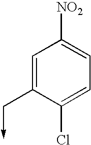 | 24 | 563, 565 [M + H]⁺ | 1.02 (m, 12H), 1.14-1.37 (m, 3H), 1.43-1.78 (m, 5H), 2.40 (m, 2H), 3.02 (m, 2H), 3.65 (s, 2H), 3.82 (s, 2H), 7.08 (d, 2H), 7.20 (dd, 1H), 7.22 (m, 1H), 7.47 (d, 1H), 7.68 (d, 1H), 7.78 (dd, 1H), 7.82 (m, 2H), 8.15 (d, 1H), 8.59 (d, 1H). |
| 91 (a) | 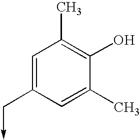 | 26 | 528, [M + H]⁺ | 1.04 (m, 12H), 1.14-1.33 (m, 3H), 1.42-1.72 (m, 5H), 2.09 (s, 6H), 2.38 (m, 2H), 3.01 (m, 2H), 3.43 (s, 2H), 3.58 (s, 2H), 6.45 (s, 2H), 7.07 (d, 2H), 7.19 (dd, 1H), 7.30 (m, 1H), 7.45 (m, 2H), 7.80 (m, 1H), 8.06 (br s, 1H), 8.62 (m, 1H). |
| 92 (d) | 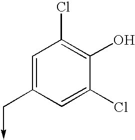 | 21 | 568, 570 [M + H]⁺ | 1.03 (m, 12H), 1.10-1.30 (m, 3H), 1.43-1.72 (m, 5H), 2.38 (m, 2H), 2.92 (m, 2H), 3.56 (s, 2H), 3.65 (s, 2H), 6.90 (s, 2H), 7.09 (d, 2H), 7.19 (m, 1H), 7.30 (m, 1H), 7.46 (d, 1H), 7.59 (s, 1H), 7.80 (m, 1H), 8.62 (d, 1H). |
| 93 (a) | 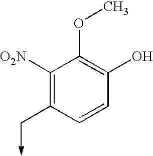 | 14 | 575 [M + H]⁺ | 1.03 (m, 12H), 1.10-1.30 (m, 3H), 1.42-1.70 (m, 5H), 2.37 (m, 2H), 2.98 (m, 2H), 3.42-3.55 (m, 4H), 3.78 (s, 3H), 6.66 (d, 1H), 7.00 (d, 1H), 7.06 (d, 2H), 7.19 (dd, 1H), 7.24 (m, 1H), 7.42 (d, 1H), 7.72 (s, 1H), 7.79 (m, 1H), 8.59 (m, H). |
| 94 (a) | 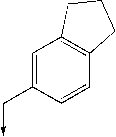 | 29 | 524 [M + H]⁺ | 1.03 (m, 12H), 1.14-1.30 (m, 3H), 1.43-1.70 (m, 5H), 1.98 (m, 2H), 2.38 (m, 2H), 2.79 (m, 4H), 3.00 (m, 2H), 3.46 (s, 2H), 3.67 (s, 2H), 6.64 (d, 1H), 6.83 (s, 1H), 7.09 (m, 3H), 7.19 (dd, 1H), 7.30 (m, 1H), 7.46 (d, 1H), 7.58 (s, 1H), 7.81 (dd, 1H), 8.62 (m, 1H). |
| 95 | 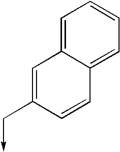 | 56 | 534 [M + H]⁺ | 0.88 (m, 6H), 1.01 (m, 6H), 1.21-1.34 (m, 3H), 1.45-1.62 (m, 3H), 1.70 (m, 2H), 2.40 (m, 2H), 2.95 (m, 2H), 3.62 (s, 2H), 3.96 (s, 2H), 7.03 (d, 2H), 7.15 (m, 2H), 7.30 (m, 1H), 7.40 (s, 1H), 7.46 (m, 3H), 7.62 (s, 1H), 7.78-7.92 (m, 4H), 8.64 (d, 1H). |
| 96 (a) | 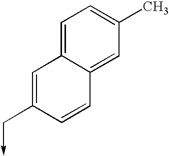 | 32 | | 0.92 (m, 6H), 1.02 (m, 6H), 1.14-1.34 (m, 3H), 1.47 (m, 1H), 1.58 (m, 2H), 1.70 (m, 2H), 2.43 (m, 5H), 2.97 (m, 2H), 3.60 (s, 2H), 3.94 (s, 2H), 7.05 (m, 3H), 7.18 (m, 1H), 7.34 (m, 3H), 7.50 (d, 1H), 7.62 (d, 2H), 7.66 (d, 1H), 7.72 (d, 1H), 7.83 (dd, 1H), 8.64 (d. 1H). |
| 97 | 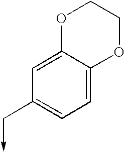 | 39 | 542 [M + H]⁺ | 1.03 (m, 12H), 1.14-1.28 (m, 3H), 1.47 (m, 1H), 1.57 (m, 2H), 1.64 (m, 2H), 2.38 (m, 2H), 2.98 (m, 2H), 3.36 (s, 2H), 3.80 (s, 2H), 4.20 (s, 4H), 6.39 (d, 1H), 6.45 (s, 1H), 6.75 (d, 1H), 7.06 (d, 2H), 7.19 (m, 1H), 7.27 (m, 1H), 7.45 (d, 1H), 7.56 (s, 1H), 7.80 (m, 1H), 8.62 (d, 1H). |
| 98 | 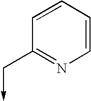 | 23 | 485 [M + H]⁺ | 1.00 (m, 12H), 1.10-1.30 (m, 3H), 1.45-1.66 (m, 5H), 2.38 (m, 2H), 2.97 (m, 2H), 3.60 (s, 2H), 3.84 (s, 2H), 7.00 (d, 1H), 7.05 (d, 2H), 7.18 (m, 1H), 7.23 (m, 2H), 7.44 (d, 1H), 7.69-7.80 (m, 3H), 8.43 (d, 1H), 8.60 (d, 1H). |
| 99 (b) | 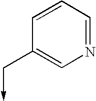 | 26 | 485 [M + H]⁺ | 1.01 (m, 12H), 1.13-1.30 (m, 3H), 1.45-1.60 (m, 3H), 1.70 (m, 2H), 2.42 (m, 2H), 2.88 (m, 2H), 3.60 (s, 2H), 3.83 (s, 2H), 7.06 (m, 3H), 7.19 (m, 2H), 7.32 (m, 1H), 7.41-7.58 (m, 3H), 7.67 (s, 1H), 7.85 (m, 1H), 8.62 (d, 1H). |
| 100 | 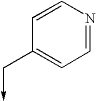 | 13 | 485 [M + H]⁺ | 0.99 (d, 6H), 1.04 (d, 6H), 1.13-1.30 (m, 3H), 1.48 (m, 1H), 1.58 (m, 2H), 1.67 (m, 2H), 2.40 (m, 2H), 2.94 (m, 2H), 3.59 (s, 2H), 3.79 (s, 2H), 6.97 (d, 2H), 7.07 (d, 2H), 7.19 (m, 1H), 7.27 (m, 1H), 7.48 (d, 1H), 7.64 (s, 1H), 7.80 (dd, 1H), 8.44 (d, 2H), 8.62 (d, 1H). |
| 101 (d) | 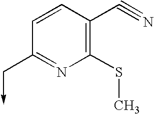 | 34 | 556 [M + H]⁺ | 0.96 (m, 6H), 0.99 (d, 6H), 1.14-1.31 (m, 3H), 1.47 (m, 1H), 1.58 (m, 2H), 1.66 (m, 2H), 2.18 (s, 3H), 2.40 (m, 2H), 3.01 (m, 2H), 3.75 (s, 2H), 3.95 (s, 2H), 6.95 (d, 1H), 7.02 (d, 2H), 7.17 (m, 1H), 7.25 (m, 1H), 7.45 (d, 1H), 7.55 (s, 1H), 7.80 (dd, 1H), 8.10 (d, 1H), 8.60 (d, 1H). |
| 102 | 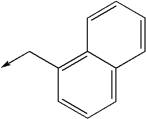 | 57 | 535 [M + H]⁺ | 0.99 (m, 6H), 1.05-1.37 (m, 9H), 1.44-1.64 (m, 3H), 1.75 (m, 2H), 2.43 (m, 2H), 3.02 (m, 2H), 3.62 (s, 2H), 4.34 (s, 2H), 7.05 (m, 3H), 7.18 (m, 1H), 7.28 (m, 1H), 7.55 (m, 3H), 7.70-7.87 (m, 3H), 8.00 (d, 1H), 8.58 (s, 1H), 8.84 (d, 1H). |
| 103 (a) | 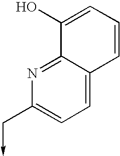 | 41 | 551 [M + H]⁺ | 0.81 (m, 6H), 0.90 (m, 6H), 1.14-1.38 (m, 3H), 1.48 (m, 1H), 1.59 (m, 2H), 1.75 (m, 2H), 2.42 (m, 2H), 2.78 (m, 2H), 3.80 (s, 2H), 4.10 (s, 2H), 7.00 (d, 2H), 7.08 (m, 1H), 7.15 (m, 1H), 7.25 (m, 2H), 7.39 (m, 2H), 7.52 (d, 1H), 7.56 (s, 1H), 7.81 (m, 1H), 8.26 (d, 1H), 8.62 (s, 1H), 9.15 (s, 1H). |
| 104 (a), (b) | 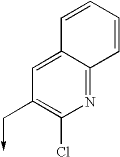 | 14 | 569, 571 [M + H]⁺ | 0.88 (m, 6H), 1.06 (d, 6H), 1.17-1.36 (m, 3H), 1.48 (m, 1H), 1.60 (m, 2H), 1.75 (m, 2H), 2.39 (m, 2H), 2.65 (s, 2H), 3.72 (s, 2H), 3.92 (s, 2H), 7.03 (d, 2H), 7.18 (m, 1H), 7.25 (m, 1H), 7.50 (d, 1H), 7.68 (m, 1H), 7.80 (m, 5H), 7.84 (s, 1H), 8.60 (d, 1H). |
| 105 | 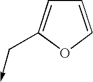 | 1 | 474 [M + H]⁺ | 1.05 (d, 6H), 1.10 (d, 6H), 1.14-1.30 (m, 3H), 1.46 (m, 1H), 1.57 (m, 2H), 1.63 (m, 2H), 2.38 (m, 2H), 3.04 (m, 2H), 3.54 (s, 2H), 3.64 (s, 2H), 6.02 (s, 1H), 6.38 (s, 1H), 7.09 (d, 2H), 7.20 (m, 1H), 7.28 (m, 1H), 7.44 (d, 1H), 7.52 (s, 1H), 7.66 (s, 1H), 7.82 (m, 1H), 8.63 (d, 1H). |
| 106 (a), (b) | 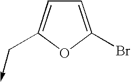 | 7 | 552, 554 [M + H]⁺ | 1.06 (d, 6H), 1.13 (d, 6H), 1.14-1.30 (m, 3H), 1.46 (m, 1H), 1.57 (m, 2H), 1.63 (m, 2H), 2.37 (m, 2H), 3.04 (m, 2H), 3.48 (s, 2H), 3.66 (s, 2H), 6.06 (s, 1H), 6.44 (s, 1H). 7.10 (d, 2H), 7.20 (m, 1H), 7.30 (m, 1H), 7.30 (m, 1H), 7.48 (d, 1H), 7.72 (s, 1H), 7.82 (m, 1H), 8.64 (d, 1H). |
| 107 | 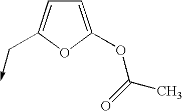 | 20 | | 1.09 (m, 12H), 1.14-1.30 (m, 3H), 1.45 (m, 1H), 1.57 (m, 2H), 1.63 (m, 2H), 2.00 (s. 3H), 2.38 (m, 2H), 3.06 (m, 2H), 3.50 (s, 2H), 3.65 (s, 2H), 5.99 (s, 1H), 6.41 (s, 1H), 7.10 (d, 2H), 7.20 (m, 1H), 7.28 (m, 1H), 7.45 (d, 1H), 7.72 (s, 1H), 7.81 (m, 1H), 8.64 (d, 1H). |
| 108 | 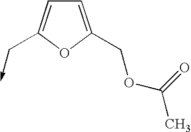 | 39 | 546 [M + H]⁺ | 1.00-1.30 (m, 15H), 1.42-1.70 (m, 5H), 2.00 (s, 3H), 2.37 (m, 2H), 3.02 (m, 2H), 3.48 (s, 2H), 3.65 (s, 2H), 4.94 (s, 2H), 5.98 (s, 1H), 6.40 (s, 1H), 7.08 (d, 2H), 7.19 (m, 1H), 7.28 (m, 1H), 7.42 (d, 1H), 7.70 (s, 1H), 7.80 (m, 1H), 8.63 (d, 1H). |
| 109 (a) | 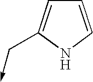 | 15 | 473 [M + H]⁺ | 0.99-1.30 (m, 15H), 1.50 (m, 3H), 1.63 (m, 2H), 2.39 (m, 2H), 2.95 (m, 2H), 3.52 (s, 2H), 3.66 (s, 2H), 5.78 (s, 1H), 5.86 (s, 1H), 6.59 (s, 1H), 7.05 (d, 2H), 7.18 (m, 1H), 7.23 (m, 1H), 7.31 (s, 1H), 7.47 (d, 1H), 7.80 (m, 1H), 8.62 (d, 1H). |
| 110 | 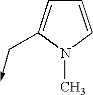 | 38 | 487 [M + H]⁺ | 1.01-1.30 (m, 15H), 1.50 (m, 3H), 1.63 (m, 2H), 2.42 (m, 2H0, 3.05 (m, 2H), 3.30 (s, 3H), 3.43 (s, 2H), 3.72 (s, 2H), 5.58 (s, 1H), 5.84 (s, 1H), 6.58 (s, 1H), 7.06 (d, 2H), 7.19 (m, 1H), 7.25 (m, 1H), 7.46 (d, 1H), 7.58 (s, 1H), 7.80 (m, 1H), 8.63 (d, 1H). |
| 111 | 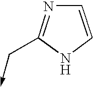 | 23 | 474 [M + H]⁺ | 0.99-1.28 (m, 15H), 1.42-1.66 (m, 5H), 2.40 (m, 2H), 2.95 (m, 2H), 3.60 (s, 2H), 3.80 (s, 2H), 6.78 (s, 1H), 6.98 (s, 1H), 7.05 (d, 2H), 7.19 (m, 1H), 7.23 (m, 1H), 7.40 (d, 1H), 7.78 (m, 1H), 8.00 (s, 1H), 8.60 (d, 1H). |
| 112 | 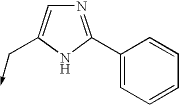 | 23 | 500 [M + H]⁺ | 1.02 (m 12H), 1.10-1.32 (m, 3H), 1.43 (m, 1H), 1.58 (m, 2H), 1.64 (m, 2H), 2.39 (m, 2H), 3.01 (m, 2H), 3.60 (s, 2H), 3.70 (s, 2H), 6.82 (s, 1H), 7.06 (d, 2H), 7.18 (m, 1H), 7.24 (m, 1H), 7.30 (m, 1H), 7.41 (m, 4H), 7.77-7.93 (m, 4H), 8.63 (d, 1H). |
| 113 | 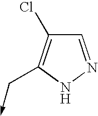 | 35 | 508, 510 [M + H]⁺ | 1.01 (m, 12H), 1.10-1.28 (m, 3H), 1.45 (m, 1H), 1.48 (m, 2H), 1.64 (m, 2H), 2.38 (m, 2H), 3.01 (m, 2H), 3.54 (s, 2H), 3.70 (s, 2H), 7.04 (d, 2H), 7.16 (m, 1H), 7.23 (m, 1H), 7.23 (m, 1H), 7.42 (m, 2H), 7.78 (m, 1H), 7.85 (s, 1H), 8.61 (d, 1H). |
| 114 | 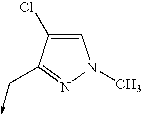 | 47 | 522, 524 [M + H]⁺ | 0.99-1.32 (m, 15H), 1.45 (m, 1H), 1.55 (m, 2H), 1.62 (m, 2H), 2.38 (m, 2H), 3.08 (m, 2H), 3.48 (s, 2H), 3.63 (s, 2H), 3.70 (s, 3H), 7.04 (d, 2H), 7.18 (m, 1H), 7.22 (m, 1H), 7.42 (d, 2H), 7.78 (m, 2H), 8.61 (s, 1H). |
| 115 | 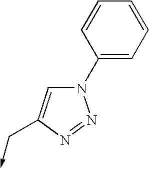 | 35 | 551 [M + H]⁺ | 0.98 (m, 12H), 1.14-1.36 (m, 3H), 1.48 (m, 1H), 1.58 (m, 2H), 1.69 (m, 2H), 2.39-2.58 (m, 2H), 2.88 (m, 2H), 3.70 (s, 2H), 3.83 (s, 2H), 7.01 (d, 2H), 7.17 (m, 1H), 7.24 (m, 1H), 7.46 (m, 2H), 7.58 (m, 3H), 7.81 (m, 3H), 8.37 (s, 1H), 8.63 (s, 1H). |
| 116 | 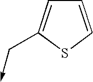 | 54 | 490 [M + H]⁺ | 1.00-1.32 (m, 15H), 1.50 (m, 3H), 1.64 (m, 2H), 2.40 (m, 2H), 3.02 (m, 2H), 3.46 (s, 2H), 3.80 (s, 2H), 6.63 (s, 1H), 6.92 (s, 1H), 7.10 (d, 2H), 7.19 (m, 1H), 7.29 (m, 1H), 7.39 (d, 1H), 7.49 (d, 1H), 7.66 (s, 1H), 7.82 (m, 1H), 8.68 (s, 1H). |
| 117 | 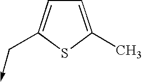 | 15 | 504 [M + H]⁺ | 1.10 (m, 12H), 1.14-1.30 (m, 3H), 1.44-1.59 (m, 3H), 1.64 (m, 2H), 2.39 (m, 5H), 3.06 (m, 2H), 3.44 (s, 2H), 3.70 (s, 2H), 6.41 (s, 1H), 6.58 (s, 1H), 7.10 (d, 2H), 7.20 (m, 1H), 7.29 (m, 1H), 7.49 (d, 1H), 7.61 (s, 1H), 7.82 (m, 1H), 8.64 (d, 1H). |
| 118 | 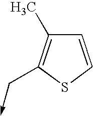 | 64 | 505 [M + H]⁺ | 1.02-1.30 (m, 15H), 1.50 (m, 3H), 1.65 (m, 2H), 1.83 (s, 3H), 2.39 (m, 2H), 3.16 (m, 2H), 3.40 (s, 2H), 3.78 (s, 2H), 6.76 (d, 1H), 7.10 (d, 2H), 7.18-7.35 (m, 3H), 7.49 (d, 1H), 7.65 (s, 1H), 7.81 (m, 1H), 8.64 (s, 1H). |
| 119 (d) | 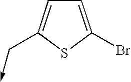 | 16 | 568, 570 [M + H]⁺ | 1.10 (m, 12H), 1.14-1.30 (m, 3H), 1.46-1.60 (m, 3H), 1.67 (m, 2H), 2.41 (m, 2H), 3.00 (m, 2H), 3.50 (s, 2H), 3.70 (s, 2H), 6.49 (s, 1H), 7.00 (d, 1H), 7.10 (d, 2H), 7.20 (m, 1H), 7.30 (m, 1H), 7.52 (d, 1H), 7.66 (s, 1H), 7.82 (m, 1H), 8.65 (d, 1H). |
| 120 (d) | 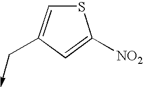 | 22 | 535 [M + H]⁺ | 1.02 (m, 12H), 1.10-1.32 (m, 3H), 1.48 (m, 1H), 1.57 (m, 2H), 1.65 (m, 2H), 2.39 (m, 2H), 2.88 (m, 2H), 3.60 (s, 2H), 3.70 (s, 2H), 7.07 (d, 2H), 7.19 (m, 1H), 7.25 (m, 1H), 7.47 (d, 1H), 7.57 (s, 1H), 7.61 (s, 1H), 7.58 (m, 2H), 8.62 (d, 1H). |
| 121 (a), (b) | 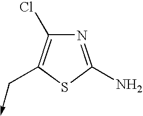 | 4 | 540, 542 [M + H]⁺ | 1.02-1.27 (m, 15H), 1.54 (m, 3H), 1.67 (m, 2H), 2.38 (m, 2H), 3.10 (m, 2H), 3.42 (s, 2H), 3.68 (s, 2H), 7.13 (d, 2H), 7.22 (m, 4H), 7.48 (d, 1H), 7.55 (s, 1H), 7.80 (m, 1H), 8.62 (d, 1H). |
| 122 | 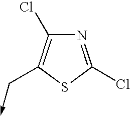 | 30 | 559, 561 [M + H]⁺ | 1.09 (d, 6H), 1.15 (d, 6H), 1.2 (m, 3H), 1.57 (m, 3H), 1.70 (m, 2H), 2.36 (m, 2H), 3.00 (m, 2H), 3.54 (s, 2H), 3.65 (s, 2H), 7.15 (d, 2H), 7.22 (d, 1H), 7.26 (m, 1H), 7.57 (d, 1H), 7.78 (s, 1H), 7.81 (m, 1H), 8.64 (d, 1H). |
| 123 (a) | 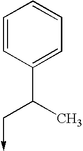 | 14 | 512 [M + H]⁺ | 0.98 (d, 3H), 1.14 (m, 15H), 1.40-1.63 (m, 5H), 2.19-2.39 (m, 4H), 2.97 (m, 1H), 3.10-3.24 (m, 2H), 3.52 (m, 2H), 6.99 (d, 2H), 7.14 (d, 2H), 7.19 (m, 5H), 7.40 (d, 1H), 7.58 (s, 1H), 7.78 (m, 1H), 8.61 (d, 1H). |
| 124 (e) | 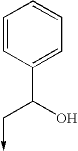 | 20 | 514 [M + H]⁺ | 1.15 (m, 15H), 1.54 (m, 5H), 2.30 (m, 2H), 2.79 (m, 1H), 3.18 (m, 2H), 3.46 (d, 2H), 4.57 (s, 2H), 7.10 (m, 4H), 7.19 (m, 3H), 7.26 (m, 2H), 7.39 (d, 1H), 7.74 (m, 2H), 8.61 (d, 1H). |
| 125 | 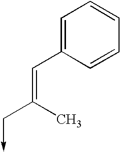 | 48 | 524 [M + H]⁺ | 1.00-1.32 (m, 15H), 1.45 (m, 1H), 1.57 (m, 5H), 1.63 (m, 2H), 2.38 (m, 2H), 2.52 (m, 2H), 3.16 (m, 2H), 3.54 (s, 2H), 5.95 (s, 1H), 7.09 (d, 2H), 7.19 (m, 4H), 7.26 (m, 1H), 7.34 (m, 2H), 7.44 (d, 1H), 7.52 (s, 1H), 7.80 (m, 1H), 8.63 (d, 1H). |
| 126 (a) | 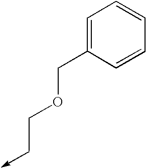 | 5 | 528 [M + H]⁺ | 1.03-1.28 (m, 15H), 1.42-1.63 (m, 5H), 2.37 (m, 2H), 2.66 (s, 2H), 3.05 (m, 2H), 3.56 (s, 2H), 4.38 (s, 2H), 7.08 (d, 2H), 7.16-7.40 (m, 7H), 7.40 (d, 1H), 7.52 (s, 1H), 7.78 (m, 1H), 8.61 (d, 1H). |

(a) the compounds were further purified using a reverse phase C18 silica gel SPE (IST®) cartridge and using an elution gradient of methanol:water.

(b) The compounds were further purified using a reverse phase C18 silica gel SPE (IST®) cartridge using acetonitrile/water with 0.1% formic acid as elution gradient.

(c) Compounds recrystallised from methanol

(d) Compounds recrystallised from diethyl ether.

(e) 3-oxo-3-phenyl-propionaldehyde (WO 9012017) was used as the starting aldehyde

### EXAMPLES 127 TO 135

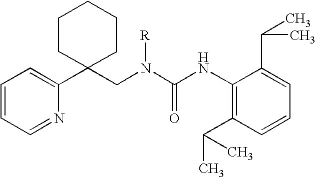

The amine from preparation 24 (0.26 mmol) in 1,2-dichloroethane (1.3 ml) was added to the appropriate aldehyde (0.26 mmol). The mixture was diluted with 1,2-dichloroethane (1.7 ml) and sodium triacetoxyborohydride (109.4 mg, 0.52 mmol) was added and the mixture was shaken for 18 hours. The reaction mixture was diluted with a saturated solution of sodium hydrogen carbonate and the phases were separated using a Whatman® 6 ml PTFE cartridge which was washed with dichloromethane (2×2 ml). 2,6-Diisopropylphenylisocyanate (52.4 mg, 0.26 mmol) in 1,2-dichloroethane (1.3 ml) was added. After 10 minutes the mixture was heated to 60° C. and the solvent was evaporated at atmospheric pressure. The residue was purified by chromatography on an 8 g Biotage® 12M silica gel column using ethyl acetate in heptanes as eluant to give the title compounds.

| Example | | Yield | LRMS (ES⁺) | |
|---|---|---|---|---|
| number | R | (%) | m/z [M + H]⁺ | ¹H NMR (DMSO-d₆, 400MHz): δ |
| 127 | 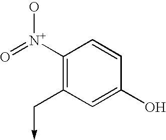 | 18 | 545 | 1.06(m, 12H), 1.16(m, 3H), 1.60(m, 5H), 2.40(m, 2H), 2.90 (m, 2H), 3.63(m, 2H), 3.80(m, 2H), 6.53(s, 2H), 6.60(s, 1H), 7.06(d, 2H), 7.16(m, 1H), 7.46(d, 1H), 7.59(s, 1H), 7.82 (dd, 1H), 7.94(d, 1H), 8.59(d, 1H). |
| 128 | 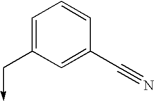 | 58 | 509 | 1.11(m, 12H), 1.12(m, 3H), 1.58(m, 5H), 2.40(m, 2H), 2.88 (m, 2H), 3.63(m, 2H), 3.80(m, 2H), 7.08(d, 2H), 7.19(m, 1H), 7.28(m, 1H), 7.37(s, 1H), 7.50(m, 2H), 7.66(s, 1H), 7.73(s, 1H), 7.81(dd, 1H), 8.59(d, 1H) |
| 129 | 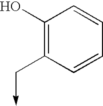 | 80 | 500 | 1.04(m, 12H), 1.19(m, 3H), 1.60(m, 5H), 2.32(m, 2H), 3.01 (m, 2H), 3.55(m, 2H), 3.75(m, 2H), 6.72(m, 2H), 6.84(m, 1H), 7.04(m, 3H), 7.18(m, 1H), 7.26(m, 1H), 7.49(d, 1H), 7.60(s, 1H), 7.80(dd, 1H), 8.61(d, 1H) |
| 130 | 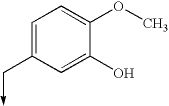 | 44 | 530 | 1.14(m, 15H), 1.60(m, 5H), 2.38(m, 2H), 3.01(m, 2H), 3.50 (m, 2H), 3.60(s, 2H), 3.77(s, 3H), 6.33(m, 1H), 6.43(s, 1H), 6.79(m, 1H), 7.06(m, 2H), 7.18(m, 1H), 7.26(m, 1H), 7.48 (m, 2H), 7.80(dd, 1H), 8.61(d, 1H), 8.78(s, 1H). |
| 131 | 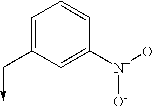 | 56 | 529 | 0.99(m, 12H), 1.19(m, 3H), 1.60(m, 5H), 2.42(m, 2H), 2.90 (m, 2H), 3.64(m, 2H), 3.94(m, 2H), 7.07(d, 2H), 7.20(m, 1H), 7.30(m, 1H), 7.41(m, 1H), 7.50(d, 1H), 7.61(dd, 1H), 7.68(s, 1H), 7.84(m, 2H), 8.10(d, 1H), 8.62(d, 1H). |
| 132 | 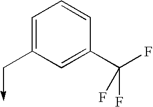 | 67 | 552 | 1.00(m, 12H), 1.20(m, 3H), 1.59(m, 5H), 2.40(m, 2H), 2.92 (m, 2H), 3.79(m, 2H), 3.91(m, 2H), 7.07(m, 2H), 7.19(m, 2H), 7.28(m, 2H), 7.53(m, 3H), 7.66(s, 1H), 7.81(dd, 1H), 8.62(d, 1H). |
| 133 | 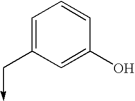 | 41 | 500 | 1.06(m, 12H), 1.21(m, 3H), 1.59(m, 5H), 2.38(m, 2H), 2.99 (m, 2H), 3.73(m, 2H), 3.88(m, 2H), 6.37(m, 2H), 6.59(d, 1H), 7.04(m, 3H), 7.17(m, 1H), 7.27(m, 1H), 7.46(d, 1H), 7.53(s, 1H), 7.81(dd, 1H), 8.63(d, 1H), 9.22(s, 1H). |
| 134 | 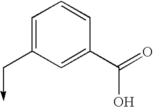 | 7 | 528 | 1.00(m, 12H), 1.22(m, 3H), 1.57(m, 5H), 2.40(m, 2H), 2.84 (m, 2H), 3.58(m, 2H), 3.83(m, 2H), 7.09(m, 4H), 7.28(m, 1H), 7.38(dd, 1H), 7.50(m, 1H), 7.59(s, 2H), 7.79(m, 2H), 8.63(d, 1H). |
| 135 | 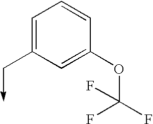 | 69 | 568 | 1.17(m, 15H), 1.57(m, 5H), 2.39(m, 2H), 2.96(m, 2H), 3.55 (m, 2H), 3.81(s, 2H), 6.93(m, 2H), 7.06(m, 2H), 7.24(m, 3H), 7.44(m, 2H), 7.68(s, 1H), 7.81(m, 1H), 8.63(m, 1H), |

### EXAMPLES 136 TO 138

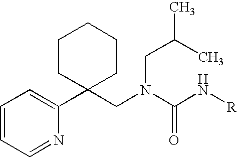

A solution of triphosgene (0.04 mmol) in dichloromethane (0.5 ml), followed by triethylamine (50 μl, 0.37 mmol) were added to a solution of the amine from preparation 59 (0.12 mmol) in dichloromethane (1.5 ml) at 0° C., and the reaction stirred for 10 minutes. A solution of the appropriate amine (RNH₂) (0.12 mmol) in dichloromethane (0.5 ml) was then added, and the mixture stirred at room temperature for 18 hours.

| Example | | LRMS (ES⁺) | Retention time |
|---|---|---|---|
| number | R | m/z [M + H]⁺ | (min) |
| 136 | 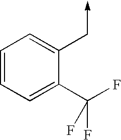 | 448.1 | 2.24 |
| 137 | 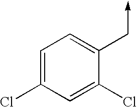 | 448.1, 450 | 2.30 |
| 138 | 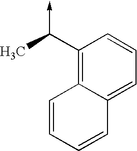 | 444.2 | 2.25 |

### EXAMPLES 139 TO 151

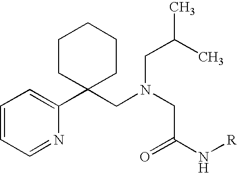

A solution of O-benzotriazol-1-yl-N,N,N′N′-tetramethyluronium hexafluorophosphate (0.13 mmol) in N′N-dimethylformamide (0.5 ml), followed by N,N-diisopropylethylamine (0.48 mol) were added to a solution of the acid from preparation 65 (0.12 mmol) in N′N-dimethylformamide (0.5 ml), and the reaction stirred at room temperature for an hour. A solution of the desired amine (RNH₂), (0.12 mmol) in N′N-dimethylformamide (0.5 ml) was added and the mixture stirred at 50° C. for 18 hours.

| Example | | LRMS (ES⁺) | Retention time |
|---|---|---|---|
| number | R | m/z [M + H]⁺ | (min) |
| 139 | 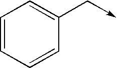 | 394.17 | 2.03 |
| 140 | 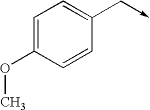 | 424.14 | 2.03 |
| 141 | 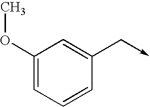 | 424.14 | 2.04 |
| 142 | 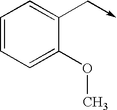 | 424.14 | 2.08 |
| 143 | 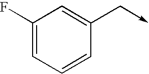 | 412.11 | 2.06 |
| 144 | 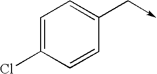 | 428.11, 430.06 | 2.15 |
| 145 | 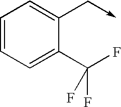 | 462.10 | 2.21 |
| 146 | 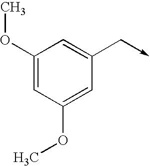 | 454.11 | 2.06 |
| 147 | 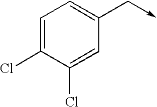 | 462.04, 464.03 | 2.26 |
| 148 | 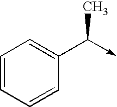 | 408.15 | 2.07 |
| 149 | 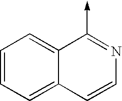 | 431.13 | 1.98 |
| 150 | 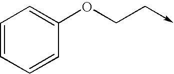 | 424.14 | 2.11 |
| 151 | 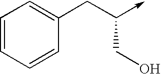 | 438.18 | 1.89 |

### EXAMPLES 152 TO 157

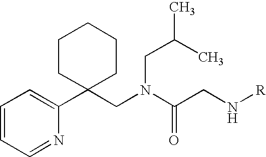

A mixture of the amine from preparation 63 (0.12 mmol) and the appropriate aldehyde (RCHO) (0.12 mmol) in methanol (1 ml) was stirred at room temperature for 18 hours. A solution of sodium borohydride (0.36 mmol) in ethanol:N′N-dimethylformamide (0.2 ml, 1:1) was added and the reaction stirred for an hour.

| | | LRMS | |
|---|---|---|---|
| | | (ES⁺) | Retention |
| Example | | m/z | time |
| number | R | [M + H]⁺ | (min) |
| 152 | 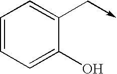 | | 1.61 |
| 153 | 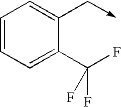 | 462.08 | 1.80 |
| 154 | 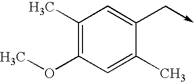 | 452.16 | 1.85 |
| 155 | 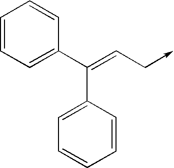 | 496.17 | 2.05 |
| 156 | 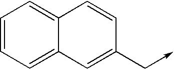 | 444.10 | 1.86 |
| 157 | 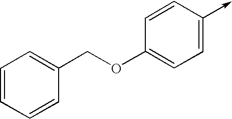 | 500.14 | 1.98 |

### EXAMPLE 158

2-Benzyl-N-(2,6-diisopropyl-phenyl]-3-(1-pyridin-2-yl-cyclohexyl)-propionamide

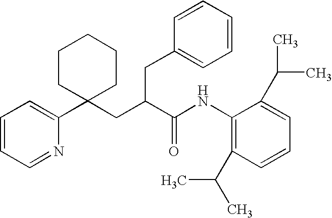

1,3-Dicyclohexylcarbodiimide (206 mg, 1 mmol) followed by diisopropyl fluorophosphate (177 mg, 1 mmol) and 2,6-diisopropylaniline (177 mg, 1 mmol) were added to a solution of the acid from preparation 71 (324 mg, 1 mmol) in ethyl acetate (10 ml), and the reaction stirred at room temperature for 18 hours. The resulting precipitate was removed by filtration, and the filtrate purified using a Biotage® column and an elution gradient of ethyl acetate:heptane (0:100 to 40:60), to afford the title compound.

¹H NMR (CDCl₃, 400 Hz): δ 0.98-1.04 (m, 6H), 1.19-1.20 (m, 6H), 1.26-1.68 (m, 9H), 2.35-2.44 (m, 6H), 2.81-2.84 (m, 2H), 6.49 (s, 1H), 6.95-8.60 (m, 12H).

LRMS (AP⁺) m/z 483.3 [M+H]⁺

### EXAMPLE 159

N-(2,6-Diisopropyl-phenyl)-2-(4-hydroxy-benzyl)-3-(1-pyridin-2-yl-cyclohexyl)-propionamide

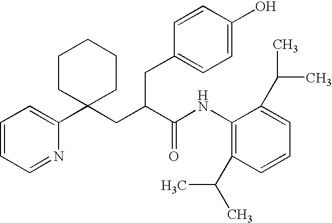

A mixture of the benzyl ether from preparation 73 (0.286 g, 0.49 mmol) and 20% Pd/C (15 mg) in methanol (15 ml) was stirred at room temperature under 1 atm of hydrogen for 18 hours. The catalyst was removed by filtration and washed with ethyl acetate (2×10 ml). The combined filtrate and washings were concentrated under reduced pressure. The residue was purified by column chromatography on silica gel using ethyl acetate:hexanes (40:60) to give a colourless oil. The oil was redissolved in ether (3 ml) and crystallization was induced by scratching the solution with a spatula. The solid material was collected and washed with 33% ether in hexanes to give the desired product as a white solid, 0.163 g.

LRMS (APCl) m/z 499.2 (M+H)⁺.

### EXAMPLE 160

3-[3-(2,6-Diisopropyl-phenyl)-1-(1-pyridin-2-yl-cyclohexylmethyl)-ureidomethyl)-benzamide

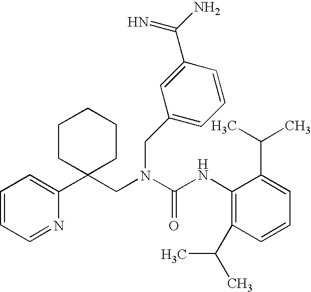

A mixture of the compound from preparation 74 (500 mg, 0.9 mmol) and 5% palladium on charcoal (100 mg) in acetic acid (20 ml) was hydrogenated at 50 psi for 2 hours. The mixture was filtered, and the filtrate evaporated under reduced pressure. The residue was purified by column chromatography on silica gel using an elution gradient of dichloromethane:methanol:acetic acid (100:0:0 to 80:20:0 to 80:20:10). The product was dissolved in ethyl acetate (100 ml), the solution washed with saturated sodium bicarbonate solution (100 ml), dried over magnesium sulphate and evaporated under reduced pressure. The product was triturated with heptane, and dried to afford the title compound, 20 mg.

¹H NMR (DMSO-d₆, 400 MHz): δ 1.02 (d, 12H), 1.15-1.35 (m, 3H), 1.48-1.62 (m, 3H), 1.72 (m, 2H), 2.45 (m, 2H), 2.85 (m, 2H), 3.65 (br s, 2H), 3.80 (br s, 2H), 7.07 (d, 2H), 7.18 (dd, 1H), 7.30 (m, 2H), 7.37 (s, 1H), 7.54 (m, 2H), 7.62 (m, 2H), 7.84 (m, 1H), 8.62 (d, 1H), 8.96 (s, 2H), 9.34 (s, 2H).

LRMS (AP⁺) m/z 526 [M+H]⁺

### EXAMPLE 161

3-[3-(2,6-Diisopropyl-phenyl)-1-(1-pyridin-2-yl-cyclohexylmethyl)-1-[3-(1-H-tetrazol-5-yl)-benzyl]-urea

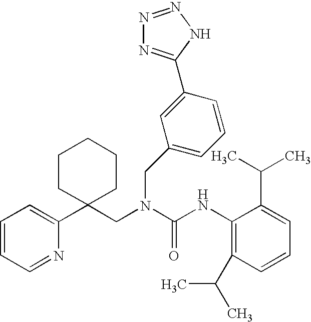

A mixture of the nitrile from example 130 (30 mg, 0.06 mmol), azidotrimethylsilane (20 mg, 0.17 mmol) and dibutyltin oxide (10 mg, 0.04 mmol) in toluene (20 ml) was heated at 95° C. for 90 minutes. The cooled mixture was quenched by the addition of hydrochloric acid (2N, 50 ml), and extracted with ethyl acetate (2×50 ml). The combined organic extracts were dried over magnesium sulphate, and evaporated under reduced pressure. The residue was purified by column chromatography on silica gel using methanol:ethyl acetate (2:98) as eluant, then recrystallised using diethyl ether:heptane to afford the title compound as a white solid, 12 mg.

¹H NMR (DMSO-d₆, 400 MHz): δ 0.90-1.34 (m, 15H), 1.45-1.62 (m, 3H), 1.70 (m, 2H), 2.38-2.50 (m, 2H), 2.97 (m, 2H), 3.60 (br s, 2H), 3.80 (br s, 2H), 6.99 (d, 1H), 7.04 (d, 2H), 7.16 (dd, 1H), 7.30 (m, 1H), 7.38 (dd, 1H), 7.49 (d, 1H), 7.62 (m, 2H), 7.84 (m, 2H), 8.64 (d 1H).

LRMS (AP⁺) m/z 552 [M+H]⁺

### EXAMPLE 162

3-(2,6-Diisopropyl-phenyl)-1-[(4-hydroxy-benzyl)-1-[1-(1-methyl-1H-imidazol-4-yl)-cyclohexylmethyl]-urea

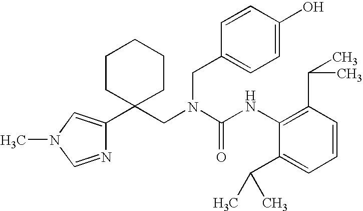

2,6-Diisopropylphenylisocyanate (0.3 g, 1.5 mmol) was added to a solution of the amine of preparation 104 (0.4 g, 1.3 mmol) in dichloromethane (30 ml) and the mixture was stirred at room temperature for 1 hour. The reaction mixture was evaporated under reduced pressure and the residue was purified by chromatography on silica gel using methanol in dichloromethane as eluant (gradient from 0:100 to 20:80). The material obtained was further purified by chromatography on silica gel using ethyl acetate in heptane (gradient from 50:50 to 100:0). The material isolated was recrystallised from dichloromethane/heptane to give the title compound as a white solid (140 mg).

M.p. 112-114° C.

¹H NMR (400 MHz, DMSO-d₆): δ 1.05 (d, 12H), 1.28 (m, 4H), 1.55 (m, 4H), 2.02 (m, 2H), 2.98 (m, 2H), 3.39 (s, 2H), 3.65 (s, 3H), 3.89 (s, 2H), 6.66 (d, 2H), 6.90 (m, 3H), 7.07 (d, 2H), 7.19 (m, 1H), 7.42 (s, 1H), 7.54 (s, 1H), 9.20 (s, 1H)

LRMS (APCl) m/z 503 [M+H]⁺

### EXAMPLES 163 TO 175

The compounds of the following tabulated examples of the general formula:

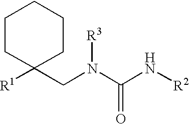

were prepared by a similar method to that of example 162 using the appropriate secondary amine and substituted phenylisocyanate.

| Example | R1 | R2 | R3 |
|---|---|---|---|
| 163 | 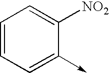 | 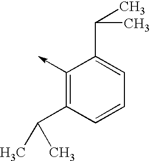 | 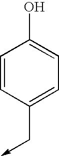 |
| 164 | 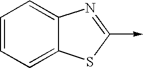 | 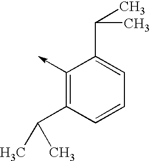 | 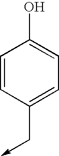 |
| 165 | 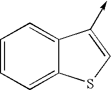 | 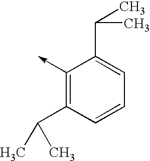 | 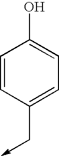 |
| 166 | 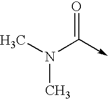 | 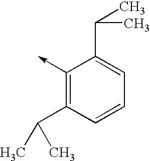 | 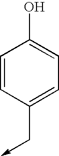 |
| 167 | 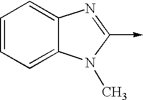 | 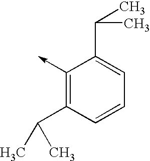 | 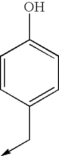 |
| 168 | 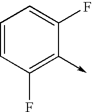 | 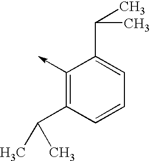 | 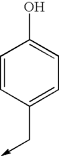 |
| 169 | 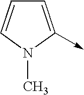 | 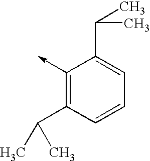 | 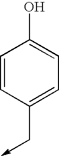 |
| 170 | 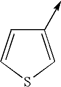 | 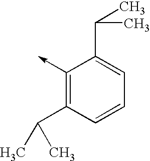 | 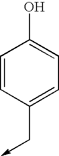 |
| 171 | 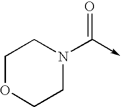 | 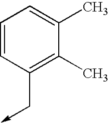 | 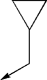 |
| 172 | 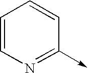 | 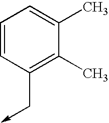 | 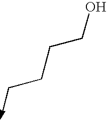 |
| 173 | 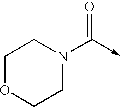 | 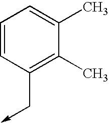 | 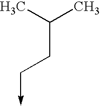 |
| 174 | 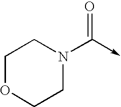 | 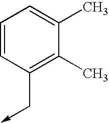 | 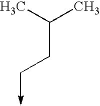 |
| 175 | 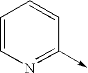 | 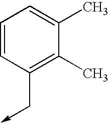 | 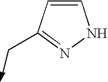 |

### EXAMPLE 163

M.p. 190-192° C.

¹H NMR (400 MHz, CDCl₃): δ 1.30 (m, 20H), 2.30 (s, 2H), 2.71 (m, 2H), 3.87 (s, 2H), 4.15 (s, 2H), 5.16 (s, 1H), 5.76 (s, 1H), 6.73 (d, 2H), 7.08 (m, 4H), 7.18 (m, 1H), 7.39 (m, 2H), 7.57 (m, 1H), 7.66 (d, 1H)

LRMS (APCl) m/z 544 [M+H]⁺

### EXAMPLE 164

M.p. 164-166° C.

¹H NMR (400 MHz, CDCl₃): δ 1.20 (m, 16H), 1.75 (m, 2H), 1.90 (m, 2H), 2.45 (m, 2H), 2.76 (m, 2H), 3.92 (m, 4H), 5.28 (s, 1H), 5.61 (s, 1H), 6.69 (d, 2H), 6.95 (d, 2H), 7.06 (d, 2H), 7.19 (m, 1H), 7.39 (m, 1H), 7.28 (m, 1H), 7.92 (d, 1H), 8.02 (d, 1H)

LRMS (APCl) m/z 556 [M+H]⁺

### EXAMPLE 165

M.p. 183-185° C.

¹H NMR (400 MHz, CDCl₃): δ 1.30 (m, 18H), 1.90 (m, 2H), 2.50 (m, 2H), 2.78 (m, 2H), 3.84 (s, 2H), 3.96 (s, 2H), 4.89 (s, 2H), 6.68 (d, 2H), 6.86 (d, 2H), 7.07 (d, 2H), 7.19 (m, 1H), 7.30 (m, 3H), 7.89 (m, 1H), 8.21 (m, 1H)

LRMS (APCl) m/z 555 [M+H]⁺

### EXAMPLE 166

M.p. 172-174° C.

¹H NMR (400 MHz, CDCl₃): δ 1.30 (m, 20H), 2.49 (m, 2H), 2.78 (m, 2H), 3.10 (s, 6H), 3.78 (s, 2H), 4.54 (s, 2H), 5.71 (s, 1H), 5.97 (s, 1H), 6.83 (d, 2H), 7.06 (d, 2H), 7.18 (m, 3H)

LCMS: m/z ES⁺ 516 [M+Na]⁺

### EXAMPLE 167

M.p. 180-182° C.

¹H NMR (400 MHz, CDCl₃): δ 0.95 (m, 12H), 1.45 (m, 8H), 2.60 (m, 2H), 2.80 (m, 2H), 3.86 (s, 2H), 4.02 (s, 3H), 4.18 (s, 2H), 5.61 (s, 1H), 6.67 (d, 2H), 6.89 (d, 2H), 7.04 (d, 2H), 7.15 (m, 1H), 7.30 (m, 2H), 7.34 (d, 1H), 7.79 (d, 1H) 8.03 (s, 1H)

LRMS (APCl) m/z 553 [M+H]⁺

### EXAMPLE 168

M.p. 181-182° C.

¹H NMR (400 MHz, CDCl₃): δ 1.30 (m, 20H), 2.79 (m, 4H), 3.78 (s, 2H), 4.14 (s, 2H), 4.97 (s, 1H), 5.56 (s, 1H), 6.75 (d, 2H), 6.85 (m, 2H), 7.06 (m, 4H), 7.21 (m, 2H)

LRMS (APCl) m/z 535 [M+H]⁺

### EXAMPLE 169

M.p. 112-115° C.

¹H NMR (400 MHz, CDCl₃): δ 1.10-170 (m, 20H), 2.35 (d, 2H), 2.81 (m, 2H), 3.59 (s, 2H), 3.75 (s, 3H), 3.96 (s, 2H), 4.86 (s, 1H), 6.07 (d, 2H), 6.53 (s, 1H), 6.76 (d, 2H), 7.07 (m, 4H), 7.19 (m, 1H)

LRMS (APCl) m/z 535 [M+H]⁺

### EXAMPLE 170

M.p. 158-160° C.

¹H NMR (400 MHz, CDCl₃): δ 1.30 (m, 20H), 2.15 (d, 2H), 2.80 (m, 2H), 3.58 (s, 2H), 3.82 (s, 2H), 4.89 (s, 1H), 5.43 (s, 1H), 6.73 (d, 2H), 7.00 (d, 2H), 7.09 (m, 4H), 7.19 (m, 1H), 7.25 (m, 1H)

LRMS (APCl) m/z 529 [M+H]⁺

### EXAMPLE 171

¹H NMR (400 MHz, CDCl₃): δ 0.14 (m, 2H), 0.53 (m, 2H), 0.96 (m, 1H), 1.35 (m, 5H), 1.62 (m, 3H), 2.28 (m, 8H), 3.08 (d, 2H), 3.59 (s, 2H), 3.68 (m, 4H), 13.74 (m, 4H), 4.41 (d, 2H), 4.81 (t, 1H), 7.10 (m, 3H)

LCMS: m/z ES⁺ 464 [M+Na]⁺

Found; C, 70.71; H, 8.83; N, 9.52 C₂₆H₃₉N₃O₃ requires; C, 70.71; H, 8.90; N, 9.52%

### EXAMPLE 172

M.p. 116-117° C.

¹H NMR (400 MHz, CDCl₃): δ 1.40 (m, 13H), 2.15 (s, 3H), 2.30 (s, 3H), 2.41 (m, 2H), 3.00 (m, 2H), 3.39 (s, 2H), 3.60 (q, 2H), 4.16 (d, 2H), 4.52 (t, 1H), 6.98 (m, 2H), 7.08 (m, 2H), 7.32 (d, 1H), 7.59 (m, 1H), 8.21 (d, 1H)

Found; C, 73.45; H, 8.91; N, 9.79; C₂₆H₃₇N₃O₂ requires; C, 73.72; H, 8.80; N, 9.92%

### EXAMPLE 173

¹H NMR (400 MHz, DMSO-d₆): δ 0.84 (d, 6H), 1.23 (m, 7H), 1.50 (m, 4H), 2.09 (d, 2H), 2.14 (s, 3H), 2.22 (s, 3H), 3.13 (m, 2H), 3.40 (s, 2H), 3.54 (m, 8H), 4.20 (d, 2H), 6.63 (t, 1H), 6.99 (m, 3H)

LCMS: m/z ES⁺ 480 [M+Na]⁺

Found; C, 70.84; H, 9.48; N, 9.14; C₂₇H₄₃N₃O₃ requires; C, 70.86; H, 9.47; N, 9.18%

### EXAMPLE 174

¹H NMR (400 MHz, DMSO-d₆): δ 0.80 (d, 6H), 1.23 (m, 5H), 1.48 (m, 3H), 1.96 (m, 1H), 2.08 (d, 2H), 2.13 (s, 3H), 2.22 (s, 3H), 3.00 (d, 2H), 3.48 (s, 2H), 3.54 (m, 8H), 4.20 (d, 2H), 6.61 (t, 1H), 6.98 (m, 3H)

LCMS: m/z ES⁺ 466 [M+Na]⁺

Found; C, 69.79; H, 9.39; N, 9.40; C₂₆H₄₁N₃O₃; 0.2H₂O requires; C, 69.93; H, 9.33; N, 9.40%

### EXAMPLE 175

¹H NMR (400 MHz, DMSO-d₆): δ 1.05-1.30 (m, 3H), 1.38-1.60 (m, 5H), 2.07 (s, 3H), 2.21 (s, 3H), 2.34 (d, 2H), 3.39 (s, 2H), 3.73 (s, 2H), 4.12 (d, 2H), 5.86 (d, 1H), 6.14 (t, 1H), 6.88 (d, 1H), 6.95 (m, 1H), 6.99 (d, 1H), 7.15 (m, 1H), 7.39 (m, 1H), 7.70 (m, 1H), 8.50 (m, 1H), 12.38 (s, 1H)

LCMS: m/z ES⁺ 432 [M+H]⁺

Found; C, 71.23; H, 7.75; N, 15.78; C₂₆H₃₃N₅O; 0.3H₂O requires; C, 71.46; H, 7.75; N, 16.03%

### EXAMPLE 176

3-(2,6-Diisopropyl-phenyl)-1-[1-(3-dimethylamino-phenyl)-cyclohexylmethyl]-1-(4-hydroxy-benzyl)-urea

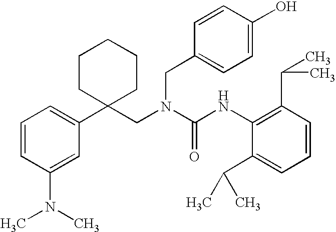

2,6-Diisopropylphenylisocyanate (0.15 g, 0.7 mmol) was added to a solution of the amine from preparation 116 (92 mg, 0.27 mmol) in dichloromethane (20 ml). The mixture was stirred at room temperature for 30 minutes and then evaporated under reduced pressure. The residue was purified by chromatography on silica gel using diethyl ether in dichloromethane as eluant (gradient from 0:100 to 10:90). The material isolated was recrystallised from diethyl ether/heptane to give the title compound as a white solid (38 mg).

M.p. 166-168° C.

¹H NMR (400 MHz, CDCl₃): δ 0.94-1.78 (m, 20H), 2.29 (d, 2H), 2.81 (m, 2H), 2.96 (s, 6H), 3.55 (s, 2H), 3.84 (s, 2H), 4.94 (s, 1H), 5.43 (s, 1H), 6.68 (m, 1H), 6.72 (d, 2H), 6.84 (m, 2H), 6.97 (d, 2H), 7.07 (d, 2H), 7.19 (m, 1H), 7.24 (m, 1H)

LRMS (APCl) m/z 542 [M+H]⁺

### EXAMPLES 177

3-(2,6-Diisopropylphenyl)-1-(4-hydroxybenzyl]-1-[1-(2-methoxyphenyl)-cyclohexylmethyl]urea

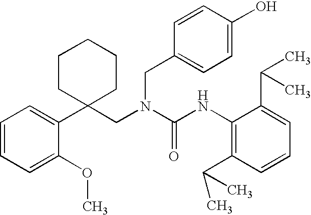

4-Hydroxybenzaldehyde (40 mg, 0.3 mol) followed by sodium triacetoxyborohydride (300 mg, 1.5 mmol) were added to a solution of the amine from preparation 22b (58 mg, 0.22 mmol) in dichloromethane (20 ml), and the reaction stirred at room temperature for 4 hours. The reaction was diluted with sodium bicarbonate solution (50 ml), and the mixture extracted with ethyl acetate (2×70 ml). The combined organic extracts were dried over magnesium sulphate and evaporated under reduced pressure.

2,6-Diisopropylphenylisocyanate (0.10 g, 0.5 mmol) was added to a solution of this amine in dichloromethane (20 ml) and the reaction was stirred at room temperature for 30 minutes. The mixture was evaporated under reduced pressure and the residue was purified by chromatography on silica gel using diethyl ether in dichloromethane as eluant (gradient from 0:100 to 10:90). The material isolated was recrystallised from diethyl ether/heptane to give the title compound as a white solid (82 mg).

M.p. 167-168° C.

¹H NMR (400 MHz, CDCl₃): δ 1.30 (m, 20H), 2.60 (s, 2H), 2.89 (m, 2H), 3.73 (m, 5H), 4.15 (s, 2H), 4.93 (s, 1H), 5.88 (s, 1H), 6.72 (d, 2H), 6.88 (d, 1H), 7.03 (m, 4H), 7.22 (m, 3H), 7.39 (d, 1H)

LRMS (APCl) m/z 529 [M+H]⁺

### EXAMPLE 178

3-(2,6-Diisopropylphenyl)-1-[1-(3-dimethylaminomethyl-phenyl]-cyclohexylmethyl]-1-(4-hydroxybenzyl)-urea

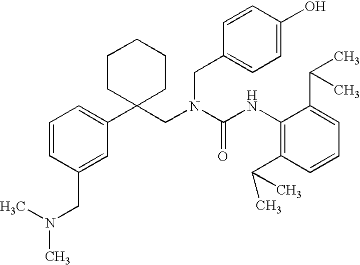

The title compound was obtained from the amine from preparation 83, 4-hydroxybenzaldehyde and 2,6-diisopropylphenylisocyanate according to the method described in example 177.

M.p. 88-90° C.

¹H NMR (400 MHz, CDCl₃): δ 1.13 (m, 20H), 2.25 (m, 8H), 2.79 (m, 2H), 3.49 (s, 2H), 3.58 (s, 2H), 3.70 (s, 2H), 5.42 (s, 1H), 6.67 (d, 2H), 6.89 (d, 2H), 7.06 (d, 2H), 7.20 (m, 3H), 7.35 (m, 2H), 7.42 (s, 1H)

LRMS (APCl) m/z 556 [M+H]⁺

### EXAMPLE 179

3-(2,6-Diisopropyl-phenyl)-1-(4-hydroxy-benzyl)-1-[1-(1H-imidazol-4-yl)-cyclohexylmethyl]-urea

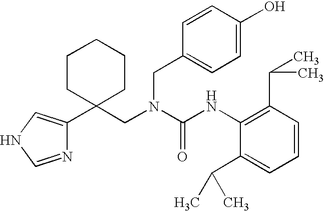

Trifluoroacetic acid (100 μl, 1.3 mmol) was added to the trityl compound of preparation 120 (50 mg, 0.07 mmol) in dichloromethane (5 ml) and the mixture was stirred at room temperature for 3 hours. The reaction mixture was partitioned between saturated sodium carbonate solution (50 ml) and ethyl acetate (100 ml). The phases were separated and the organic phase was dried over magnesium sulphate and evaporated under reduced pressure. The residue was purified by chromatography on silica gel using methanol in dichloromethane as eluant (gradient from 5:95 to 10:90). The material obtained was recrystallised from ethyl acetate/heptane to give the title compound as a white solid (6 mg).

M.p. 185-188° C.

¹H NMR (400 MHz, DMSO-d₆): δ 1.00-1.65 (m, 20H), 2.05 (m, 2H), 2.90-3.05 (m, 2H), 3.40 (m, 2H), 3.60-3.85 (m, 2H), 6.67 (m, 2H), 6.85 (m, 3H), 7.04 (m, 2H), 7.19 (m, 1H), 7.37, 7.51 (2xs, 1H), 7.62 (s, 1H), 9.20, 9.23 (2xs, 1H), 11.80, 11.85 (br s, 1H)

LRMS (APCl) m/z 489 [M+H]⁺

### EXAMPLE 180

1-Benzyl-3-(2,6-diisopropyl-phenyl)-1-[1-(1-methyl-1H-tetrazol-5-yl)-cyclohexylmethyl]-urea

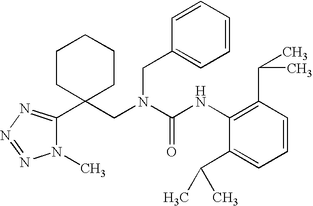

The carboxylic acid of preparation 92 (1.05 g, 5 mmol) was dissolved in ethyl acetate (100 ml) and N,N′-dicyclohexylcarbodiimide (1.03 g, 5 mmol) and pentafluorophenol (0.92 g, 5 mmol) were added. The mixture was stirred at room temperature for 4 hours and then was cooled to 4° C. for 2 hour. The reaction mixture was filtered and benzylamine (0.64 g, 6 mmol) was added to the filtrate. The mixture was stirred for 16 hours and the solvent was evaporated under reduced pressure. The residue was purified by chromatography on reverse phase silica gel using methanol in water as eluant (50:50). The residue was dissolved in tetrahydrofuran (20 ml) under a nitrogen atmosphere. Borane-methyl sulphide complex (1M in tetrahydrofuran, 3 ml, 3 mmol) was added and the mixture was heated under reflux for 16 hours. The reaction mixture was cooled to room temperature and methanol (10 ml) was added dropwise over 15 minutes. The mixture was acidified to pH 2 with 4N hydrochloric acid and then heated under reflux for 30 minutes. The reaction mixture was evaporated under reduced pressure and then co-evaporated with methanol (2×30 ml). The residue was partitioned between dichloromethane and 10% sodium hydroxide solution. The organic phase was dried over potassium carbonate and evaporated under reduced pressure. The residue was purified by chromatography on reverse phase silica gel using methanol in water as eluant (gradient from 0:100 to 100:0). The material obtained was dissolved in N,N-dimethylformamide (5 ml) and 2,6-diisopropylphenylisocyanate (18 mg, 0.09 mmol) was added. The mixture was stirred at room temperature for 1 hour and the solvent was evaporated under reduced pressure. The residue was purified by chromatography on reverse phase silica gel using methanol in water as eluant (65:35). The material isolated was recrystallised from methanol to give the title compound (13 mg).

M.p. 150-152° C.

¹H NMR (400 MHz, CDCl₃): δ 0.97 (m, 12H), 1.53 (m, 4H), 1.74 (m, 4H), 2.38 (m, 2H), 4.51 (d, 2H), 3.89 (s, 2H), 4.22 (s, 3H), 4.51 (s, 2H), 5.45 (s, 1H), 7.02 (d, 2H), 7.12 (m, 1H), 7.32 (m, 3H), 7.39 (m, 2H)

Found; C, 71.43; H, 8.23; N, 17.08; C₂₉H₄₀N₆O requires C, 71.28; H, 8.25; N, 17.19%

### EXAMPLE 181

1-Benzyl-3-(2,6-diisopropyl-phenyl)-1-(1-pyrimidin-2-yl-cyclohexylmethyl)-urea

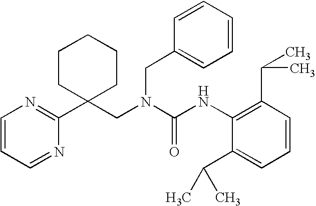

Sodium hydride (60% in mineral oil, 0.54 g, 13.4 mmol) was suspended in dimethylsulphoxide (40 ml) under an argon atmosphere. A solution of 2-pyrimidineacetonitrile (0.8 g, 6.7 mmol) and 1,5-dibromopentane (1.55 g, 6.7 mmol) in dimethylsulphoxide (20 mol) and diethyl ether (40 ml) was added and the mixture was stirred at room temperature for 16 hours. Propan-2-ol (10 ml) and water (20 ml) were added and the reaction mixture was partitioned between water and ethyl acetate. The organic phase was separated, dried over magnesium sulphate and evaporated under reduced pressure. The residue was purified by chromatography on silica gel using ethyl acetate in heptane as eluant (gradient from 0:100 to 40:60).

The material isolated was dissolved in a saturated solution of ethanolic ammonia (20 ml) and was added to Raney® Nickel (1 g) that had been washed with water to pH 7, then washed with ethanol and then suspended in ethanol (70 ml). The mixture was hydrogenated at 60 psi for 2 hours and then was filtered through Celite®. The filter cake was washed with methanol and the combined organic filtrates were evaporated under reduced pressure.

The residue was dissolved in 1,2-dichloroethane (20 ml) and sodium triacetoxyborohydride (424 mg, 2 mmol) and benzaldehyde (106 mg, 1 mmol) were added. The mixture was stirred at room temperature for 20 hours and then was partitioned between saturated sodium hydrogen carbonate solution and dichloromethane (50 ml). The aqueous phase was extracted with dichloromethane (50 ml) and the combined organic phases were dried over magnesium sulphate and evaporated under reduced pressure.

The residue was dissolved in tetrahydrofuran (20 ml) and 2,6-diisopropylphenylisocyanate (203 mg, 1 mmol) was added. The mixture was stirred at room temperature for 16 hours and then evaporated under reduced pressure. The residue was purified by chromatography on silica gel using ethyl acetate in heptane as eluant (35:65) and the material obtained was recrystalised from ethyl acetate/heptane to give the title compound (195 mg).

M.p. 112.5-113.5° C.

¹H NMR (400 MHz, CDCl₃): δ 1.10 (m, 16H), 1.72 (m, 4H), 2.61 (d, 2H), 2.79 (m, 2H), 3.85 (s, 2H), 4.12 (s, 2H), 5.70 (s, 1H), 7.08 (m, 4H), 7.25 (m, 5H), 8.73 (d, 2H)

LCMS: m/z ES⁺ 485 [M+H]⁺

Found; C, 77.05; H, 8.42; N, 11.64; C₃₁H₄₀N₄O requires C, 76.82; H, 8.32; N, 11.55%

### EXAMPLE 182

1-Benzyl-3-(2,6-diisopropyl-phenyl)-1-[1-(2-nitro-phenyl)-cyclohexylmethyl]-urea

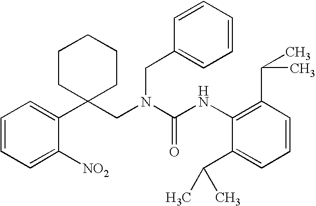

A solution of benzaldehyde (1.02 ml, 0.10 mmol) and the amine from preparation 102 (2.34 g, 10 mmol) in toluene (100 ml) was heated under reflux using a Dean and Stark apparatus, to remove residual water. The reaction mixture was evaporated under reduced pressure and the residue dissolved in ethanol (50 ml). Sodium borohydride (567 mg, 15 mmol) was added and the reaction stirred at room temperature for 3 hours. The reaction was quenched by the addition of water, the mixture concentrated under reduced pressure and the residue partitioned between dichloromethane and 15% sodium hydroxide solution. The layers were separated, the aqueous phase extracted with further dichloromethane and the combined organic solutions dried over potasium carbonate and evaporated under reduced pressure. This product was dissolved in tetrahydrofuran (30 ml) and 2,6-diisopropylphenylisocyanate (2.03 g, 10.0 mmol) was added. The mixture was stirred at room temperature for 24 hours and then was evaporated under reduced pressure. The residue was purified by chromatography on silica gel using ethyl acetate in heptane (2:98) and the product crystallised from ether to afford the title compound as a solid, 2.86 g.

¹H NMR (400 MHz, CDCl₃): δ 0.80-1.38 (m, 15H), 1.48-1.73 (m, 5H), 2.20-2.40 (s, 2H), 2.75 (s, 2H), 3.43 (s, 2H), 4.22 (s, 2H), 5.71 (m, 1H), 7.06 (d, 2H), 7.15-7.45 (m, 8H), 7.56 (m, 1H), 7.66 (m, 1H)

LRMS (APCl) m/z 528 [M+H]⁺

Found; C, 74.85; H, 7.85; N, 7.96; C₃₃H₄₁N₃O₃ requires; C, 75.11; H, 7.83; N, 7.96%

### EXAMPLE 183

3-(2,6-Diisopropyl-phenyl)-1-isobutyl-1-(3-methyl-2-phenyl-butyl)-urea

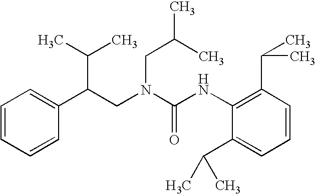

The title compound was obtained from the amino compound of preparation 117 and 2,6-diisopropylphenylisocyanate in 13% yield following the procedure described in example 162.

M.p. 67-69° C.

¹H NMR (400 MHz, CDCl₃): δ 0.73 (d, 3H), 0.88 (d, 6H), 1.03 (d, 3H), 1.20 (m, 12H), 1.93 (m, 2H), 2.55 (m, 1H), 2.80 (m, 1H), 2.95 (m, 3H), 3.51 (m, 1H), 3.95 (m, 1H), 5.39 (s, 1H), 7.11 (d, 2H), 7.24 (m, 6H)

LRMS (APCl) m/z 528 [M+H]⁺

### EXAMPLE 184

1-Cyclopropylmethyl-3-(2,3-dimethyl-benzyl)-1-(1-pyridin-2-yl-cyclohexylmethyl)-urea

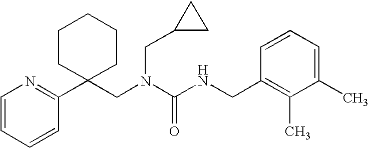

Triphosgene (100 mg, 0.4 mmol) in dichloromethane (3 ml) and triethylamine (430 μl, 3.1 mmol) were added to the amine of preparation 111 (250 mg, 1.02 mmol) in dichloromethane (10 ml) at 0° C. and the mixture was stirred at 0° C. for 10 minutes. 2,3-Dimethylbenzylamine (140 mg, 1.02 mmol) in dichloromethane (3 ml) was added and the mixture was warmed to room temperature. The reaction mixture was stirred at room temperature for 16 hours and then evaporated under reduced pressure. The residue was dissolved in ethyl acetate (20 ml) and was washed with sodium carbonate solution (2×20 ml). The organic layer was separated, dried over sodium sulphate and evaporated under reduced pressure. The residue was purified by chromatography on silica gel using methanol in dichloromethane as eluant (gradient from 0:100 to 2:98). The material isolated was further purified by chromatography on silica gel using ethyl acetate in pentane as eluant (gradient from 0:100 to 20:80). The residue was crystallised from ethyl acetate/pentane and isolated by filtration to give the title compound as a white solid (84 mg).

M.p. 102-103° C.

¹H NMR (400 MHz, CDCl₃): δ 0.08 (m, 2H), 0.20 (m, 2H), 0.88 (m, 1H), 1.26 (m, 4H), 1.59 (m, 4H), 2.19 (s, 3H), 2.30 (s, 3H), 2.40 (m, 2H), 2.80 (d, 2H), 3.52 (s, 2H), 4.20 (d, 2H), 4.47 (m, 1H), 6.99 (m, 2H), 7.10 (m, 2H), 7.32 (d, 1H), 7.58 (m, 1H), 8.31 (d, 1H)

LCMS: m/z ES⁺ 428 [M+Na]⁺

Found; C, 76.90; H, 8.67; N, 10.26; C₂₆H₃₅N₃O requires; C, 77.00; H, 8.70; N, 10.36%

### EXAMPLES 185 TO 186

The compounds of the following tabulated examples of the general formula:

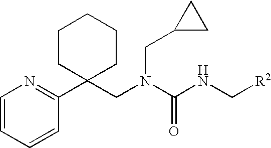

were prepared by a similar method to that of example 184 using the secondary amine of preparation 111, triphosgene and the appropriate benzylamine.

| Ex- | | |
|---|---|---|
| am- | | |
| ple | R² | Data |
| 185 | 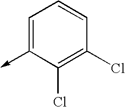 | ¹H NMR(400MHz, CDCl₃):δ0.08(m, 2H), 0.22(m, 2H), 0.90(m, 1H), 1.24(m, 4H), 1.58(m, 4H), 2.40(m, 2H), 2.80(d, 2H), 3.50(m, 2H), 4.29(d, 2H), 4.92(m, 1H), 7.01(m, 1H), 7.17(m, 2H), 7.30(m, 2H), 7.60(m, 1H), 7.50(d, 1H) |
| 186 | 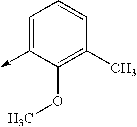 | ¹H NMR(400MHz, CDCl₃):δ0.04(m, 2H), 0.040(m, 2H), 0.84(m, 1H), 1.20 (m, 4H), 1.56(m, 4H), 2.10(s, 3H), 2.42 (d, 2H), 2.78(s, 2H), 3.48(s, 2H), 3.82(s, 3H), 4.20(d, 2H), 4.47(s, 1H), 6.78(m, 2H), 6.98(m, 1H), 7.10(m, 1H), 7.30(d, 1H), 7.58(m, 1H), 8.32(d, 1H) LRMS(APCI)m/z 422[M + H]⁺ |

### EXAMPLE 187

3-(2,6-Diisopropylphenyl)-1-(4-hydroxybenzyl)-1-(1-thiophen-2-yl-cyclohexylmethy)urea

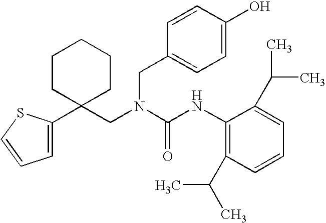

The compound from preparation 54 (130 mg, 0.66 mmol) followed by sodium triacetoxyborohydride (500 mg, 2.5 mmol) were added to a solution of 4-hydroxybenzaldehyde (80 mg, 0.66 mmol) in dichloromethane (30 ml), and the reaction stirred at room temperature for 18 hours. Saturated aqueous sodium carbonate solution (50 ml) was added, and the mixture extracted with ethyl acetate (2×60 ml). The combined organic solutions were dried (MgSO₄) and evaporated under reduced pressure. The product was dissolved in dichloromethane (20 ml), 2,6-diisopropylphenylisocyanate (150 mg, 0.74 mmol) added and the reaction stirred at room temperature for 20 minutes. The solution was evaporated under reduced pressure and the residue was purified by column chromatography on silica gel using an elution gradient of heptane:ethyl acetate (90:10 to 50:50). The product was recrystallised from dichloromethane/heptane to afford the title compound as a white solid, 220 mg.

¹H NMR (400 MHz, CDCl₃): δ 0.80-1.85 (m, 20H), 2.10-2.22 (m, 2H), 2.81 (m, 2H), 3.62 (s, 2H), 3.85 (s, 2H), 5.16 (s, 1H), 5.47 (s, 1H), 6.72-6.94 (m, 2H), 6.90-7.30 (m, 8H).

LRMS (APCl) m/z 505 [M+H]⁺

### EXAMPLE 188

3-(2,6-Diisopropylphenyl)-1-(4-hydroxybenzyl)-1-[1-(5-methoxy-pyridin-2-yl)-cyclohexylmethyl]urea

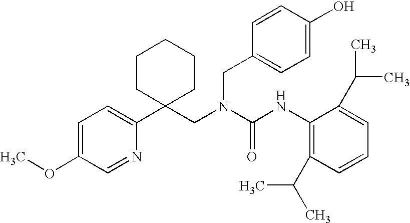

Sodium triacetoxyborohydride (211 mg, 1.0 mmol) was added to a solution of 4-hydroxybenzaldehyde (61 mg, 0.5 mmol) and 1-(5-methoxy-2-pyridinyl)cyclohexanemethanamine (WO 9807718) (109 mg, 0.5 mmol) in 1,2-dichloroethane (10 ml), and the reaction stirred at room temperature for 18 hours. Saturated aqueous sodium carbonate solution was added, and the mixture extracted with dichloromethane. The combined organic solutions were dried (MgSO₄) and evaporated under reduced pressure. The product was dissolved in dichloromethane (10 ml), 2,6-diisopropylphenylisocyanate (102 mg, 0.5 mmol) added and the reaction stirred at room temperature for 15 minutes. The solution was evaporated under reduced pressure and the residue was purified by column chromatography on silica gel using heptane:ethyl acetate (90:10) as eluant. The product was further purified by column chromatography on reverse phase silica gel, using methanol:water (80:20) as eluant to afford the title compound as a crystalline solid.

¹H NMR (400 MHz, CDCl₃): δ 0.90-1.80 (m, 20H), 2.42 (m, 2H), 2.75 (m, 2H), 3.68 (s, 2H), 3.86 (s, 3H), 3.90 (s, 2H), 5.69 (s, 1H), 5.92 (s, 1H), 6.69 (d, 2H), 6.97 (d, 2H), 7.06 (m, 2H), 7.19 (m, 2H), 7.37 (d, 1H), 8.32 (d, 1H).

LRMS (APCl) m/z 530 [M+H]⁺

Preparation 1

3-Cyano-N-ethyl-benzamide

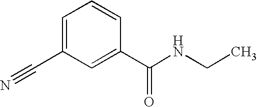

Ethylamine in tetrahydrofuran (1.0M, 27 ml, 27.0 mmol) was diluted with tetrahydrofuran (100 ml) and cooled to 0° C. 3-Cyanobenzoyl chloride (2.0 g, 12.10 mmol) in tetrahydrofuran (10 ml) was added dropwise and the mixture stirred for 30 minutes at 0° C., then warmed to room temperature. The mixture was concentrated under reduced pressure and the solid obtained was isolated by filtration, washed with water, and dried under vacuum at 40° C. for 18 hours to give the title compound (1.64 g).

LRMS (APCl) m/z 174.9 [M+H]⁺

Preparations 2-6

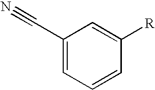

The compounds of the following tabulated preparations of the general formula shown, were prepared by a similar method to that of preparation 1 using 3-cyanobenzoylchloride and the appropriate starting amines.

| | | MS data |
|---|---|---|
| Preparation number | R | (LRMS (APCI) m/z) |
| 2 | 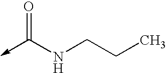 | 188.9 [M + H]⁺ |
| 3 | 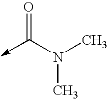 | 174.9 [M + H]⁺ |
| 4 | 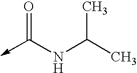 | 188.9 [M + H]⁺ |
| 5 | 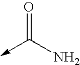 | 146.9 [M + H]⁺ |
| 6 | 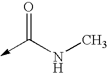 | 160.9 [M + H]⁺ |

Preparation 7

2,2,2-Trifluoroethanesulfonic Acid (4-cyanophenyl) Amide

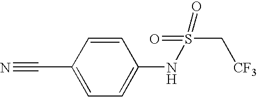

To a solution of 4-cyanoaniline (3.02 g, 25.58 mmol) in anhydrous dichloromethane (25 ml) at 0° C. was added pyridine (2.3 ml, 28.44 mmol) and then 2,2,2-trifluoroethanesulfonyl chloride in dichloromethane (10 ml) slowly. The reaction mixture was stirred at 0° C. for 15 minutes and then at room temperature for 30 minutes. Water (100 ml) and ethyl acetate (100 ml) were added. The aqueous layer was extracted with ethyl acetate (2×100 ml). The combined organic layers were dried over magnesium sulphate and evaporated under reduced pressure. The solid obtained was triturated with a dichloromethane/ether mixture (25:75) and was isolated by filtration to give the title compound (5.25 g).

Preparation 8

N-Ethyl-3-formyl-benzamide

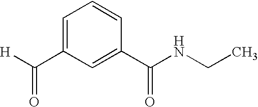

The nitrile from preparation 1 (1.64 g, 9.41 mmol) was combined with nickel aluminium alloy (9.4 g), formic acid (95%, 133 ml) and water (10 ml). The suspension was heated at reflux under nitrogen for 18 hours and the hot reaction mixture was filtered through Celite®. The filter cake was washed with formic acid, the filtrate was concentrated under reduced pressure, and the residue basified with 1N sodium hydroxide solution to pH=9. The basic solution was extracted with ethyl acetate (4×50 ml), dried over magnesium sulphate, filtered and evaporated under reduced pressure. The residue was purified by column chromatography on silica gel eluting with ethyl acetate:hexanes (40:60) to give the title compound 1.30 g.

LRMs (APCl) m/z 177.9 (M+H)⁺.

Preparation 9-13

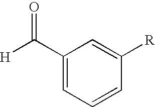

The compounds of the following tabulated preparations of the general formula shown were prepared by a similar method to that of preparation 8 using the appropriate nitrile starting material.

| Preparation | | MS data |
|---|---|---|
| number | R | (LRMS (APCI) m/z) |
| 9 | 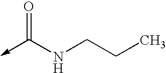 | 192.0 [M + H]⁺ |
| 10 | 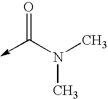 | 177.9 [M + H]⁺ |
| 11 | 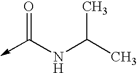 | 192.0 [M + H]⁺ |
| 12 | 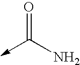 | 149.9 [M + H]⁺ |
| 13 | 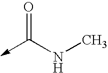 | 164.9 [M + H]⁺ |

Preparation 14

N-(4-Formyl-phenyl)-methanesulphonamide

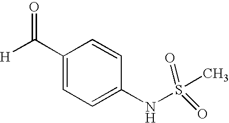

The title compound was obtained from N-(4-cyanophenyl)-methanesulfonamide, following a similar procedure to that described in preparation 8.

LRMS (APCl) m/z 374.1 [M+H]⁺

Preparation 15

2,2,2-Trifluoro-ethanesulphonic Acid N-(4-formyl-phenyl)-amide

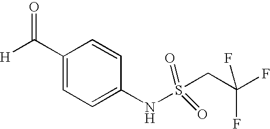

The title compound was obtained from the nitrile from preparation 7, following a similar procedure to that described in preparation 8.

LRMS (APCl) m/z 267.9 [M+H]⁺

Preparation 16

2-Oxo-2,3-dihydro-benzoxazole-5-carbaldehyde

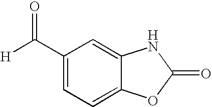

A suspension of 2,3-dihydro-2-oxo-5-benzoxazolecarbonitrile [(1.67 g, 10.45 mmol), prepared according to the procedure described in WO 9912903] and Ni—Al alloy (8.87 g) in a mixture of 88% formic acid (125 ml) and water (10 ml) was heated at 120° C. for 4 hours. The reaction mixture was filtered while hot through a pad of Celite®. The residue was washed with ethyl acetate (3×40 ml) and the filtrate and washings were combined and concentrated under reduced pressure. The residue was dissolved in ethyl acetate (200 ml) and water (100 ml). After stirring at room temperature for 10 minutes, the ethyl acetate layer was collected. The aqueous layer was extracted with ethyl acetate (2×50 mL). The organic layers were combined and dried over magnesium sulphate, filtered and concentrated under reduced pressure. The solid residue was triturated with a 1:1 mixture of ethyl acetate/ether (20 ml) to give the title compound as a pale yellow solid, 1.07 g

LRMS (APCl) m/z 163.9 [M+H]⁺.

Preparation 17

1-Hydroxymethyl-cyclohexanecarbonitrile

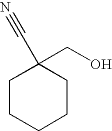

Diisopropylamine (6.8 ml, 42.3 mmol) in tetrahydrofuran (40 ml) was added to a solution of n-butyl lithium (20 ml of 2.5M solution in hexanes, 50 mmol) in tetrahydrofuran (160 ml) under an argon atmosphere at −40° C. The mixture was stirred for 25 minutes and then cooled to −76° C. Cyclohexanecarbonitrile (4.8 ml, 40 mmol) in tetrahydrofuran (100 ml) was added dropwise over 1 hour, with the reaction temperature being maintained below −74° C. The reaction mixture was stirred at −76° C. for 1 hour, after which gaseous formaldehyde prepared by pyrolysis of paraformaldehyde (2.4 g, 80 mmol) was added under argon pressure. The reaction mixture was stirred at −40° C. for 3 hour then aqueous tetrahydrofuran was added and the solvent was evaporated under reduced pressure. The residue was diluted with 2M hydrochloric acid (200 ml) and was extracted into dichloromethane (2×200 ml). The combined dichloromethane layers were dried over magnesium sulphate and evaporated under reduced pressure. The residue was purified by chromatography on silica gel using an elution gradient of methanol:dichloromethane (0:100 to 1.5:98.5) to give the title compound (4.2 g).

Preparation 18

1-Methoxymethyl-cyclohexanecarbonitrile

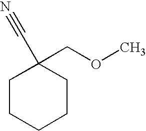

A solution of the alcohol from preparation 17 (4.2 g, 30.2 mmol) and iodomethane (7.16 ml, 115 mmol) in tetrahydrofuran (50 ml) was added over 1 hour to a suspension of sodium hydride (60% in mineral oil, 4.6 g, 115 mmol) in tetrahydrofuran (75 ml) at 0° C. The mixture was stirred for 18 hours at room temperature. Propan-2-ol was added and then water and the mixture was added to water (100 ml). The aqueous solution was extracted into dichloromethane (2×150 ml). The combined dichloromethane layers were dried over magnesium sulphate and were evaporated under reduced pressure. The residue was purified by chromatography on silica gel using an elution gradient of ethyl acetate:heptane (0:100 to 10:90) to give the title compound (5.39 g).

¹H NMR (CDCl₃, 400 MHz): δ 1.23 (m, 3H), 1.67 (m, 5H), 1.98 (m, 2H), 3.36 (s, 2H), 3.42 (s, 3H).

Preparation 19

(1-Methoxymethyl-cyclohexyl)-methylamine

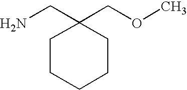

A solution of the nitrile from preparation 18 (1.1 g, 7.2 mmol) in ethanol (50 ml) was added to Raney® nickel (1.1 g) that had been washed with water (2×15 ml) and with ethanol (2×10 ml). Ethanolic ammonia (10 ml) was added and the mixture was hydrogenated at atmospheric pressure at 30° C. for 18 hours. The mixture was filtered through kieselguhr and evaporated under reduced pressure to give the title compound (1.12 g)

¹H NMR (400 MHz, CDCl₃): δ 1.20-1.60 (m, 2H), 2.62 (s, 2H), 3.23 (s, 2H), 3.32 (s, 3H).

LRMS (ES⁺) m/z 158 [M+H]⁺

Preparation 20

4-{[(1-Methoxymethyl-cyclohexylmethyl)-amino]-methyl}-phenol

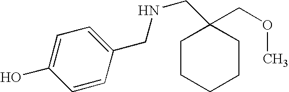

4-Hydroxybenzaldehyde (120 mg, 1 mmol) and the amine from preparation 19 (160 mg, 1 mmol) were dissolved in dichloromethane (10 ml), sodium triacetoxyborohydride (500 mg, 2.5 mmol) was added and the mixture was stirred at room temperature for 18 hours. The reaction mixture was partitioned between sodium carbonate solution (100 ml) and ethyl acetate (70 ml). The ethyl acetate layer was dried over magnesium sulphate and evaporated under reduced pressure to give the title compound (300 mg).

Preparation 21

1-(2-Methoxy-phenyl)-cyclohexanecarbonitrile

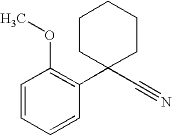

Sodium hydride (60% in oil, 4.4 g, 110 mmol) under a nitrogen atmosphere was washed with heptane (3×50 ml) and was suspended in dimethylsulphoxide (200 ml). To this suspension was added a solution of (2-methoxyphenyl)acetonitrile (7.36 g, 50 mmol) and 1,5-dibromopentane (12.64 g, 55 mmol) in diethyl ether (50 ml) over 1 hour. The mixture was stirred for 18 hours after which propan-2-ol (5 ml) and water (300 ml) were added. The aqueous solution was extracted with ethyl acetate (3×200 ml) and the combined ethyl acetate layers were dried over magnesium sulphate and evaporated under reduced pressure. The residue was purified by chromatography on silica gel using ethyl acetate:heptane (8:92) as eluant, then recrystallised from ethyl acetate/heptane to afford the title compound (5.94 g).

¹H NMR (CDCl₃, 400 MHz): δ 1.20-1.30 (m, 1H), 1.72-1.92 (m, 7H), 2.35-2.40 (m, 2H), 3.92 (s, 3H), 6.94 (m, 2H), 7.28-7.34 (m, 2H).

LCMS (ES⁺) m/z 216 [M+H]⁺

Preparation 22a

[1-(2-Hydroxy-phenyl)-cyclohexyl]-methylamine

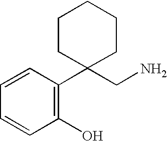

and

Preparation 22b

[1-(2-Methoxy-phenyl)-cyclohexyl]-methylamine Hydrochloride

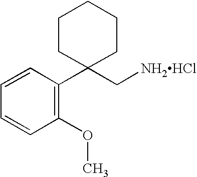

A solution of the nitrile from preparation 21 (4.31 g, 20 mmol) in ether was added over 30 minutes to a mixture of aluminium chloride (2.67 g, 20 mmol) and lithium aluminium hydride (1M solution in diethyl ether, 20 ml, 20 mmol) in diethyl ether (100 ml) at 0° C. under a nitrogen atmosphere. The mixture was heated at reflux for 2 hours and then cooled to 0° C. Water (25 ml) containing tetrahydrofuran (25 ml) was added, followed by sodium hydroxide solution (15% weight/volume, 50 ml) and the mixture was stirred for 15 minutes. The organic layer was separated, dried over potassium carbonate and evaporated under reduced pressure. The residue was purified by chromatography on silica gel using ethyl acetate:methanol:0.88 ammonia (100:0 to 95:5:0.5) to provide the title compound of preparation 22a, 1.13 g.

¹H NMR (400 MHz, CDCl₃): δ 1.50, (m, 6H), 1.74 (m, 2H), 2.19 (m, 2H), 2.99 (s, 2H), 6.79 (m, 1H), 7.15 (m, 2H), 7.19 (dd, 1H).

Further elution, provided [1-(2-methoxy-phenyl)-cyclohexyl]-methylamine, 2.8 g. A sample of this (219 mg) was dissolved in dichloromethane (1 ml) and hydrogen chloride (4M in 1,4-dioxane, 0.25 ml, 1 mmol) was added. The solvent was evaporated under reduced pressure and the residue was triturated with diethyl ether to give the title compound (218 mg).

¹H NMR (400 MHz, CDCl₃): δ 1.57, (m, 8H), 2.32 (m, 2H), 3.22 (m, 2H), 3.84 (s, 3H), 6.89 (m, 2H), 7.22 (m, 1H), 7.30 (d, 1H), 7.54 (m, 2H)

Preparation 23

1-[Pyridin-2-yl]-cyclohexanecarbonitrile

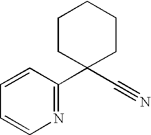

Sodium hydride (60% in oil, 7.46 g, 186 mmol) under a nitrogen atmosphere was washed with heptane (3×50 ml) and was suspended in dimethylsulphoxide (200 ml). A solution of (pyridin-2-yl)acetonitrile (10.0 g, 84.7 mmol) and 1,5-dibromopentane (21.44 g, 93.2 mmol) in diethyl ether (100 ml) was added to the mixture over an hour, maintaining the temperature below 25° C. The mixture was stirred for an hour and the diethyl ether was evaporated under reduced pressure. The residue was diluted with water and the aqueous mixture was extracted with ethyl acetate. The combined organic extracts were washed with brine, dried over magnesium sulphate and evaporated under reduced pressure. The residue was purified by chromatography using an elution gradinet of ethyl acetate:heptane (0:100 to 20:80) to afford the title compound (13.03 g).

¹H NMR (CDCl₃, 400 MHz): δ 1.36 (m, 1H), 1.84 (m, 5H), 1.99-2.19 (m, 4H), 7.22 (m, 1H), 7.60 (d, 1H), 7.72 (m, 1H), 8.60 (d, 1H).

LRMS (ES⁺) m/z 295 [M+Na]⁺

Preparation 24

(1-Pyridin-2-yl-cyclohexyl)-methylamine

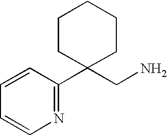

The nitrile from preparation 23 (13.03 g, 70.1 mmol), was added to washed (neutral) Raney® nickel (catalytic) and saturated ethanolic ammonia (200 ml). The mixture was hydrogenated (50 psi, 30° C.) for 48 hours. The mixture was filtered through Kieselguhr and the filter cake was washed with methanol. The filtrate was evaporated under reduced pressure to afford the title compound (12.88 g).

¹H NMR (CDCl₃, 400 MHz): δ 1.46 (m, 8H), 2.28 (m, 2H), 2.81 (s, 2H), 7.11 (s, 1H), 7.34 (m, 1H), 7.66 (s, 1H), 8.63 (s, 1H).

Preparation 25

Benzyl-(1-pyridin-2-yl-cyclohexylmethyl)-amine

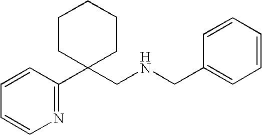

Sodium triacetoxyborohydride (23.6 g, 111.35 mmol) was added portionwise to a solution of the amine from preparation 24 (10.59 g, 55.67 mmol) and benzaldehyde (5.94 ml, 58.46 mmol) in 1,2-dichloroethane (400 ml) and the mixture was stirred for 18 hours. The reaction mixture was partitioned between brine and dichloromethane and the dichloromethane layer was evaporated under reduced pressure. The residue was purified by chromatography on silica gel using methanol:ethyl acetate (5:95) to give the title compound (12.46 g).

¹H NMR (CDCl₃, 400 MHz): δ 1.30-1.60 (m, 6H), 1.68 (m, 2H), 2.25 (m, 2H), 2.79 (s, 2H), 3.63 (s, 2H), 7.05-7.25 (m, 6H), 7.37 (d, 1H), 7.62 (m, 1H), 8.60 (d, 1H).

LRMS (ES⁺) m/z 281 [M+Na]⁺

Preparation 26

1-Aminomethyl-cyclohexanecarboxylic Acid Ethyl Ester

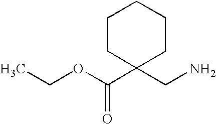

A solution of 1-cyano-cyclohexanecarboxylic acid ethyl ester (Bioorg. Med. Chem. Lett. 1999, 9(3), 369) (4.22 g, 3.3 mmol) and ethanolic ammonia (2M) was added to Raney® nickel that had been washed with water until pH neutral and then with ethanol. The mixture was hydrogenated (45 psi, 30° C.) for 16 hours and then was filtered through kieselguhr. The solvent was evaporated to give the title compound (4.33 g).

Preparation 27

1-[(4-Hydroxy-benzylamino)-methyl]-cyclohexanecarboxylic Acid Ethyl Ester

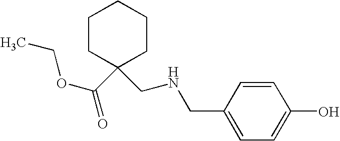

The amine from preparation 26 (250 mg, 1.4 mmol) was added to a solution of 4-hydroxybenzaldehyde (180 mg, 1.5 mmol) in dichloromethane (20 ml) and sodium triacetoxyborohydride (1 g, 5 mmol) was added. The mixture was stirred for 18 hours and then diluted with a saturated solution of sodium hydrogen carbonate (100 ml). The aqueous layer was extracted with ethyl acetate (2×100 ml) and the combined organic solutions were dried over magnesium sulphate and evaporated under reduced pressure to give the title compound (300 mg).

LRMS (APCl) m/z 292 [M+H]⁺

Preparation 28

1-Pyridin-2-yl-cyclohexanecarbaldehyde

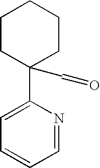

The nitrile from preparation 23 (60 g, 0.32 mol) was dissolved in a mixture of formic acid (300 ml) and water (120 ml) and was heated to 100° C. Raney® nickel (60 g) was added and the mixture was stirred at 100° C. for 20 minutes. The mixture was filtered hot and the filter cake was washed with water (200 ml), ethyl acetate (2×500 ml) and water (200 ml). The combined aqueous layers were extracted with ethyl acetate (250 ml) and the combined ethyl acetate layers were evaporated under reduced pressure. The residue was dissolved in dichloromethane (500 ml), washed with a saturated solution of sodium hydrogen carbonate (2×750 ml), dried over magnesium sulphate and evaporated under reduced pressure. The residue was purified by chromatography on silica gel using hexanes:ethyl acetate (50:50) as eluant to give the title compound (26.8 g).

Preparation 29

4-[(1-Pyridin-2-yl-cyclohexylmethylimino)-methyl]-phenol

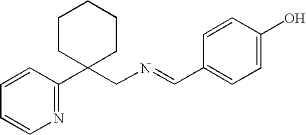

The amine from preparation 24 (9.5 g, 50 mmol) was dissolved in toluene (75 ml) containing 4-hydroxybenzaldehyde (6.1 g, 50 mmol) and heated at reflux under a Dean and Stark trap for 2 hours. The solvent was evaporated under reduced pressure. The residue was suspended in diethyl ether (15 ml) and filtered to give the title compound (13 g).

Preparation 30

4-{[(1-Pyridin-2-yl-cyclohexylmethyl)-amino]-methyl}-phenol

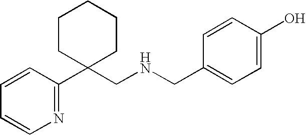

The imine from preparation 29 (13 g, 44.2 mmol) was dissolved in ethanol (100 ml) and cooled to 0° C. Sodium borohydride (1.89 g, 50 mmol) was added portionwise over 1 hour, and stirred for a further 3 hours at 0° C. The solvent was evaporated under reduced pressure maintaining the temperature below 30° C. and the residue partitioned between ethyl acetate and saturated sodium hydrogen carbonate solution. The ethyl acetate phase was dried over magnesium sulphate and evaporated under reduced pressure. The residue was dissolved in diethyl ether and cooled to −20° C. for 18 hours. The solid obtained was isolated by filtration to give the title compound (10.8 g).

¹H NMR (DMSO-d₆, 400 MHz): δ 1.24-1.56 (m, 6H), 1.63 (m, 2H), 2.05 (m, 2H), 3.10 (m, 2H), 3.98 (m, 2H), 6.78 (d, 2H), 7.24 (d, 2H), 7.38 (m, 1H), 7.58 (d, 1H), 7.90 (m, 1H), 8.56 (d, 1H), 8.67 (br s, 1H).

LRMS (ES⁺) m/z 297 [M+H]⁺

Preparation 31

(4-Methoxy-benzyl)-(1-pyridin-2-yl-cyclohexylmethyl)-amine

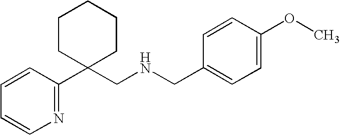

The amine from preparation 24 (6.34 g, 33.3 mmol) was dissolved in toluene (75 ml) containing 4-methoxybenzaldehyde (4.53 g, 33.3 mmol) and heated at reflux using a Dean and Stark trap for 16 hours. The solvent was evaporated under reduced pressure and the residue dissolved in ethanol (80 ml). The mixture was cooled to 0° C. and sodium borohydride (1.26 g, 33 mmol) was added portionwise over 5 minutes. The mixture was stirred for 2.5 hours at 0° C. The solvent was evaporated under reduced pressure maintaining the temperature below 30° C. The residue was dissolved in ethyl acetate and was washed twice with saturated sodium hydrogen carbonate solution, with brine, dried over magnesium sulphate and evaporated under reduced pressure. The residue was purified by chromatography on silica gel using an elution gradient of ethyl acetate:heptane (0:100 to 100:0) to give the title compound (7.08 g).

¹H NMR (CDCl₃, 400 MHz): δ 1.25-1.69 (m, 8H), 2.2-2.28 (m, 2H), 2.73 (s, 2H), 3.58 (s, 2H), 3.78 (s, 3H), 6.78 (d, 2H), 7.05 (d, 2H), 7.06-7.10 (m, 1H), 7.33-7.35 (m, 1H), 7.59-7.63 (m, 1H), 8.59-8.6 (m, 1H).

LRMS (AP⁺) m/z 311 [M+H]⁺

Preparation 32

1-Isocyanatomethyl-2,3-dimethyl-benzene

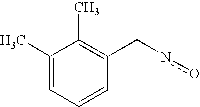

A solution of 2,3-dimethylbenzylamine (2 g, 14.79 mmol) and triethylamine (2.99 g, 29.59 mmol) in dichloromethane was added to a solution of triphosgene (1.75 g, 5.92 mmol) in dichloromethane (20 ml) at 0° C. over 30 minutes. The mixture was stirred at 0° C. for an hour and then stirred for a further 2 hours at room temperature. The mixture was filtered and the filtrate was evaporated under reduced pressure to give the title compound (2.45 g).

Preparation 33

4-Phenyl-tetrahydro-pyran-4-carbonitrile

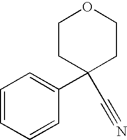

Sodium hydride (60% in oil, 0.44 g, 11 mmol) under a nitrogen atmosphere was washed with heptane (3×15 ml) and was suspended in dimethylsulphoxide (5 ml). To this suspension was added a solution of benzonitrile (0.59 g, 5 mmol) and bis(2-bromoethyl)ether (1.35 g, 5.25 mmol) in dimethylsulphoxide (5 ml). The mixture was stirred for 18 hours after which methanol (2 ml) was added and the mixture was poured onto water (200 ml). The aqueous solution was extracted with ethyl acetate (3×30 ml). The combined ethyl acetate layers were washed with citric acid solution (10% weight/volume), sodium carbonate solution (10% weight/volume), brine then dried over magnesium sulphate and evaporated under reduced pressure to give the title compound (0.80 g).

¹H NMR (CDCl₃400 MHz): δ 2.04-2.19 (m, 4H), 3.92 (m, 2H), 4.10 (m, 2H), 7.34-7.51 (m, 5H)

Preparation 34

(4-Phenyl-tetra hydro-pyran-4-yl)-methylamine

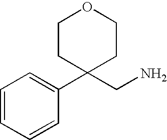

The nitrile from preparation 33 (602 mg, 70.1 mmol), was dissolved in saturated ethanolic ammonia (50 ml) and added to washed (neutral) Raney® nickel (650 mg) and hydrogenated (50 psi, 30° C.) for 24 hours. The mixture was filtered and the filtrate was evaporated under reduced pressure. The residue was purified by chromatography on silica gel using an elution gradient of methanol:dichloromethane (o:100 to 20:80) to give the title compound (502 mg).

¹H NMR (CDCl₃ 400 MHz): δ 1.85 (m, 2H), 2.46 (m, 2H), 2.81 (s, 2H), 3.55 (m, 2H), 3.81 (m, 2H), 7.32 (m, 5H).

Preparation 35

3-Methyl-2-phenylbutylamine

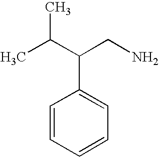

Lithium aluminium hydride (1M solution in diethyl ether, 2 ml, 2 mmol) was added over 15 minutes to a solution of 3-methyl-2-phenylbutyronitrile (250 mg, 1.6 mmol) in diethyl ether (30 ml). The mixture was stirred for 15 minutes and methanol (5 ml) was added. The solvent was evaporated under reduced pressure and the residue was partitioned between ethyl acetate (50 ml) and water (50 ml). The ethyl acetate layer was dried over magnesium sulphate and evaporated under reduced pressure to give the title compound (230 mg).

LRMS (AP⁺) m/z 164 [M+H]⁺

Preparation 36

4-[(3-Methyl-2-phenyl-butylamino)-methyl]-phenol

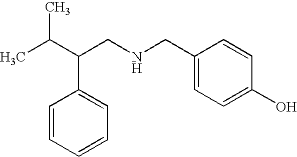

To a solution of the amine from preparation 35 (115 mg, 0.7 mmol) in dichloromethane was added 4-hydroxybenzaldehyde (100 mg, 0.8 mmol) and sodium triacetoxyborohydride (600 mg, 3 mmol). The mixture was stirred at room temperature for an hour and then was diluted with saturated sodium carbonate solution (100 ml). The aqueous mixture was extracted with ethyl acetate (2×50 ml). The combined ethyl acetate layers were dried over magnesium sulphate and evaporated under reduced pressure to give the title compound (220 mg).

LRMS (AP⁺) m/z 270 [M+H]⁺

Preparation 37

N-Ethyl-3-{[(1-pyridin-2-yl-cyclohexylmethyl)-amino]-methyl}-benzamide

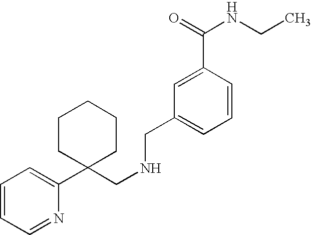

The aldehyde from preparation 8 (0.50 g, 2.82 mmol), the amine from preparation 24 (0.54 g, 2.82 mmol) and sodium triacetoxyborohydride (1.26 g, 5.93 mmol) were dissolved in tetrahydrofuran (50 ml) and the reaction was stirred under nitrogen for 18 hours, after which additional sodium triacetoxyborohydride (0.3 g, 1.40 mmol) was added. The reaction was stirred for 18 hours and saturated sodium hydrogen carbonate solution (20 ml) was added. The mixture was stirred for 30 minutes and the aqueous layer was extracted with ethyl acetate (4×20 ml), the combined ethyl acetate layers were dried over magnesium sulphate and evaporated under reduced pressure. The residue was purified by chromatography on silica gel eluting with an elution gradient of ethyl acetate:hexanes (75:25 to 100:0) to give the title compound (0.350 g).

LRMS (AP⁺) m/z 352.2 [M+H]⁺

Preparations 38-50

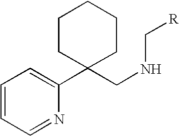

The compounds of the following tabulated preparations of the general formula shown were prepared by a similar method to that of preparation 37 using the amine from preparation 24 and the appropriate aldehyde.

| Preparation | | MS |
|---|---|---|
| number | R | (LRMS (APCI) m/z) |
| 38 | 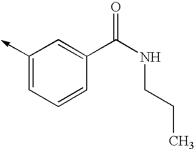 | 366 [M + H]⁺ |
| 39 | 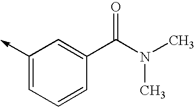 | 352 [M + H]⁺ |
| 40 | 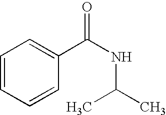 | 366 [M + H]⁺ |
| 41 | 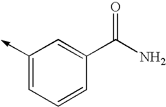 | 324 [M + H]⁺ |
| 42 | 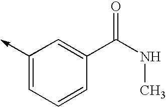 | 338 [M + H]⁺ |
| 43 | 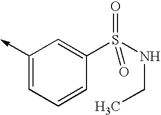 | |
| 44 | 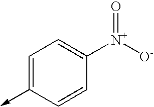 | Mp 66.2-66.8° C. |
| 45 | 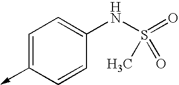 | 374 [M + H]⁺ |
| 46 | 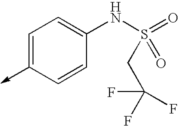 | 442 [M + H]⁺ |
| 47 (i) | 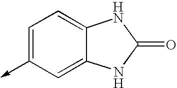 | 337 [M + H]⁺ |
| 48 (ii) | 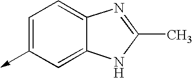 | 335 [M + H]⁺ |
| 49 (iii) | 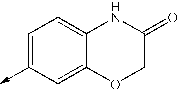 | 352 [M + H]⁺ |
| 50 | 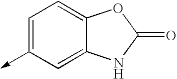 | 338 [M + H]⁺ |

(i) 2,3-dihydro-2-oxo-1H-benzimidazole-5-carboxaldehyde starting material, prepared as in WO 09941241
(ii) 2-methyl-1H-benzimidazole-5-carboxaldehyde starting material prepared as in WO 0037473
(iii) 3,4-dihydro-3-oxo-2H-1,4-benzoxazine-7-carboxaldehyde starting material, as prepared in WO 9745419.

Preparation 51

4-{2-[(1-Pyridin-2-yl-cyclohexylmethyl)-amino]-ethyl}-phenol

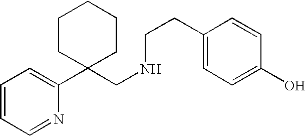

The title compound was obtained, following the procedure described in preparation 37, using the aldehyde from preparation 28 and 4-(2-aminoethyl)phenol.

LRMS (APCl) m/z 311 [M+H]⁺

Preparation 52

1-Pyridin-2-yl-cycloheptanecarbonitrile

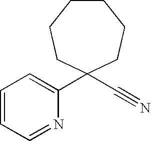

A solution of 2-pyridylacetonitrile (1.18 g, 10 mmol) and 1,6-dibromohexane (2.64 g, 10.5 mmol) in tetrahydrofuran (250 ml) was added dropwise to cooled (−78° C.) solution of lithium bis(trimethylsilyl)amide (22 ml, 1M in tetrahydrofuran, 22 mmol) in tetrahydrofuran (250 ml), over 6 hours. Once the addition was complete, the mixture was allowed to stir at room temperature for 72 hours. The reaction was quenched by the addition of saturated ammonium chloride solution (10 ml), then diluted with water (250 ml), and sodium hydroxide solution (5N, 25 ml). The phases were separated, the aqueous layer extracted with ethyl acetate (250 ml) and the combined organic layers washed with brine, dried over magnesium sulphate and evaporated under reduced pressure. The crude product was purified by column chromatography on silica gel using a Biotage® cartridge, and an elution gradient of heptane: ethyl acetate (100:0 to 90:10) to afford the title compound, 400 mg.

¹H NMR (CDCl₃, 300 MHz) δ 1.60-1.90 (m, 8H), 2.10-2.30 (m, 4H), 7.19-7.23 (m, 1H), 7.61 (d, 1H), 7.68-7.73 (m, 1H), 8.57-8.60 (m, 1H).

LRMS (AP⁺) 201.17 [M+H]⁺

Preparation 53

(1-Pyridin-2-yl-cycloheptyl)-methylamine

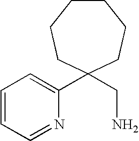

A mixture of the nitrile from preparation 52 (700 mg, 3.72 mmol) and pre-washed Raney® nickel (1 ml, 50% suspension in water, washed with water until pH neutral, then washed with ethanol (30 ml)) in ethanolic ammonia (100 ml, 2M) was hydrogenated at 50 psi and 30° C. for 24 hours. The mixture was filtered and the filtrate evaporated under reduced pressure to afford the title compound, 736 mg.

¹H NMR (CDCl₃, 300 MHz) δ: 1.45-2.00 (m, 10H), 2.20-2.30 (m, 2H), 2.90 (s, 2H), 7.11 (m, 1H), 7.33 (m, 1H), 7.64 (m, 1H), 8.60 (m, 1H).

LRMS: m/z (AP⁺) 205.17 [M+H]⁺

Preparation 54

(1-Thiophene-2-yl-cyclohexyl)-methylamine Hydrochloride

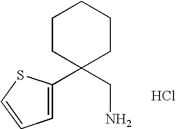

A mixture of 1-thiophen-2-yl-cyclohexanecarbonitrile (28.9 g, 163 mmol) (WO 9202481) and Raney® Nickel (10 g) in methanolic ammonia (200 ml) was hydrogenated. The mixture was filtered carefully, and the filtrate concentrated under reduced pressure. The residue was distilled using Kugelrohr apparatus, and the product converted to it's hydrochloride salt.

Mp-165-166° C.

Microanalysis found: C, 55.16; H, 7.52; N, 6.49. C₁₀H₁₅NS; HCl requires C, 55.15; H, 7.41; N, 6.43%.

Preparation 55

2-[1-(4-Hydroxybenzylamino-methyl)-cyclohexyl]-phenol

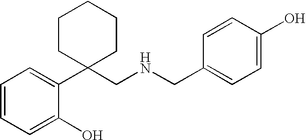

Sodium triacetoxyborohydride (500 mg, 2.5 mmol) was added to a solution of the amine from preparation 22 (100 mg, 0.5 mmol) and 4-hydroxybenzaldehyde (60 mg, 0.5 mmol) in dichloromethane (20 ml) and the reaction stirred at room temperature for 4 hours. The mixture was washed with aqueous sodium bicarbonate solution (50 ml) and the aqueous extracted with ethyl acetate (2×70 ml). The combined organic solutions were dried over magnesium sulphate, evaporated under reduced pressure and the product then dried in vacuo, to afford the title compound.

LRMS (AP⁺) m/z 312 [M+H]⁺

Preparation 56

1-(2-Amino-3-isopropyl-phenyl)-ethanone

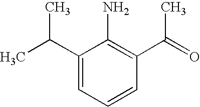

2-Isopropylaniline (4.74 g, 35 mmol) in toluene (30 ml) was added over 15 minutes to boron trichloride (1M solution in p-xylene, 40 ml, 40 mmol) at 0° C. The mixture was stirred for 5 minutes and acetonitrile (5.8 ml, 104 mmol) was added. Within 3 minutes, aluminium chloride (4.6 g, 34.6 mmol) was added and the mixture was warmed to room temperature over 20 minutes. The mixture was heated at reflux for 16 hours and then cooled to 0° C. 1N Hydrochloric acid (60 ml) was added dropwise and the mixture was heated at reflux for 1 hour. The mixture was cooled to room temperature, filtered and extracted with ethyl acetate (3×10 ml). The combined ethyl acetate layers were washed with water (×2), brine and dried over magnesium sulphate. The solvent was evaporated under reduced pressure to give the title compound (3.4 g).

LRMS (AP⁺) m/z 178 [M+H]⁺

Preparation 57

2-(2-Amino-3-isopropyl-phenyl)-propan-2-ol

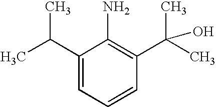

Methyl magnesium chloride (3M in tetrahydrofuran, 10 ml, 30 mmol) was added over 30 minutes to the ketone from preparation 56 (1.73 g 10 mmol) in tetrahydrofuran (20 ml) at 0° C. under a nitrogen atmosphere. The rate of addition was adjusted to maintain the reaction temperature below 5° C. The mixture was stirred at 0° C. for 1 hour, and then water was added dropwise at a rate that kept the temperature below 10° C. Ethyl acetate (40 ml) was added and the mixture was washed with a saturated solution of sodium hydrogen carbonate, dried over magnesium sulphate and evaporated under reduced pressure to give the title compound.

Preparation 58

2-Isopropenyl-6-isopropyl-phenylamine

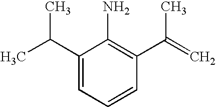

Toluene-4-sulphonic acid hydrate (1.9 g, 10 mmol) was added to the alcohol from preparation 57 (1.93 g, 10 mmol) in 1,2-dichloroethane (30 ml) and was heated at reflux. The reaction mixture was washed with 5N sodium hydroxide solution and the organic layer was dried over magnesium sulphate and evaporated under reduced pressure. The residue was purified by chromatography on a 40 g Biotage® column using heptane as eluant to give the title compound (2.1 g).

LRMS (AP⁺) m/z 176 [M+H]⁺

Preparation 59

Isobutyl-(1-pyridin-2-yl-cyclohexylmethyl)-amine

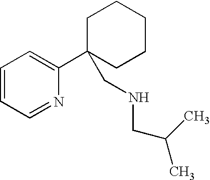

Sodium triacetoxyborohydride (42.4 g, 0.2 mol) was added to a solution of the amine from preparation 24 (19 g, 0.1 mol) and isobutyraldehyde (7.2 g, 0.1 mol) in 1,2-dichloroethane (200 ml) at 0° C. The mixture was warmed to room temperature and stirred for 18 hours. The mixture was diluted with 1,2-dichloroethane and washed with 0.5M sodium hydroxide solution and brine, then dried over magnesium sulphate and evaporated under reduced pressure. The residue was purified by chromatography on silica gel using an elution gradient of ethyl acetate:heptane (0:100 to 100:0) to give the title compound (16.66 g).

¹H NMR (CDCl₃, 400 MHz): δ 0.83 (d, 6H), 1.37-1.55 (m, 4H), 1.63 (m, 2H), 1.78 (m, 2H), 1.93 (m, 1H), 2.18 (m, 2H), 2.42 (d, 2H), 2.93 (s, 2H), 5.30-5.50 (br s; 1H), 7.17 (m, 1H), 7.39 (d, 1H), 7.68 (m, 1H), 8.54 (d, 1H).

LRMS (ES⁺) m/z 247 [M+H]⁺

Preparation 60

4-Methylpiperazine-1-acetic Acid Benzyl Ester

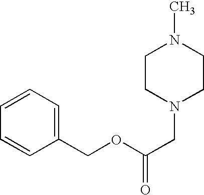

A solution of benzyl 2-bromoacetate (11.45 g, 50 mmol) in dichloromethane (20 ml) was added to an ice-cooled solution of N-methylpiperazine (5.0 g, 50 mmol) and triethylamine (6.95 ml, 50 mmol) in dichloromethane (40 ml), and the reaction then stirred at room temperature for 16 hours. The mixture was concentrated under reduced pressure and the residue dissolved in ethyl acetate, then washed with aqueous saturated sodium bicarbonate soluiton and brine, then dried over magnesium sulphate and evaporated under reduced pressure. The crude product was purified by column chromatography on silica gel using an elution gradient of heptane:ethyl acetate:methanol (20:80:0 to 0:100:0 to 0:90:10) to afford the title compound, 2.3 g.

¹H-NMR (CDCl₃, 400 MHz) δ: 2.29 (s, 3H), 2.49 (m, 4H), 2.62 (m, 4H), 3.26 (s, 2H), 5.16 (s, 2H), 7.30-7.38 (m, 5H).

LRMS (ES⁺) m/z 249.5 [M+H⁺]

Preparation 61

4-Methylpiperazine-1-acetic Acid

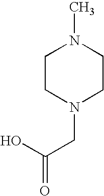

A mixture of the benzyl ester from preparation 60 (2.3 g, 9.3 mmol) and 10% palladium on charcoal (230 mg) in ethanol (50 ml), methanol (20 ml) and ethyl acetate (25 ml) was hydrogenated at 50 psi for 2 hours. The mixture was filtered through Celite®, and the filtrate evaporated under reduced pressure to afford the title compound as an off-white solid, 1.37 g.

¹H NMR (CDCl₃, 400 MHz): δ 2.16 (s, 3H), 2.33 (m, 4H), 2.62 (m, 4H), 3.12 (s, 2H), 6.90-7.30 (br s, 1H).

LRMS (ES⁺) m/z 159.4 [M+H⁺]

Preparation 62

{[Isobutyl-(1-pyridin-2-yl-cyclohexylmethyl)-carbamoyl]-methyl]-carbamic Acid tert-Butyl Ester

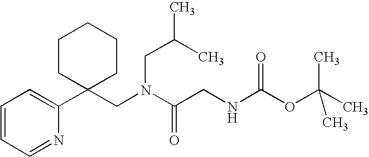

1,3-Diisopropylcarbodiimide (6.42 g, 50.9 mmol) followed by 1-hydroxybenzotriazole hydrate (690 mg, 5.09 mmol) were added to an ice-cooled solution of N-(tert-butylcarbonyl)glycine (7.85 g, 44.8 mmol) in dichloromethane (400 ml), and the mixture-stirred for 1 hour. A solution of the amine from preparation 59 (10.0 g, 40.7 mmol) in dichloromethane (100 ml) was added, and the reaction stirred at room temperature for 24 hours. The mixture was evaporated under reduced pressure and the residue dissolved in ethyl acetate (400 ml), washed with water (3×200 ml), brine (2×200 ml), then dried over magnesium sulphate and evaporated under reduced pressure. The crude product was purified by column chromatography on silica gel using hexane:ethyl acetate (66:34) as eluant to afford the title compound, 16.0 g.

¹H NMR (CDCl₃, 270 MHz) δ (rotamers): 0.63 (d, 6H), 1.23-1.43 (m, 4H), 1.44 (s, 9H), 1.56-1.68 (m, 7H), 3.62 (br s, 2H), 3.65 (d, 2H), 5.50 (br s, 1H), 7.11-7.16 (m, 1H), 7.32-7.35 (m, 1H), 7.61-7.67, 8.60-8.63 (m, 1H).

LRMS m/z 404 [M+H⁺]

Preparation 63

2-Amino-N-isobutyl-N-(1-pyridin-2-yl-cyclohexylmethyl)-acetamide

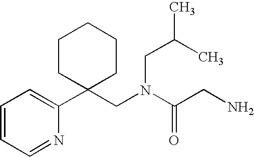

A solution of hydrochoric acid in dioxan (60 ml, 4M) was added to a solution of the protected amine from preparation 62 (16.0 g, 39.7 mmol), and the reaction stirred at room temperature for 18 hours. The resulting precipitate was removed by filtration, the solid dissolved in water (100 ml), and washed with dichloromethane (100 ml). The aqueous solution was basified to pH 11, then extracted with ethyl acetate (2×200 ml). The combined organic solutions were dried over magnesium sulphate and evaporated under reduced pressure to afford the title compound.

¹H NMR (CDCl₃, 270 MHz) δ (rotamers): 0.64, 0.73 (2xd, 6H), 2.05-2.15 (m, 2H), 2.30-2.34 (m, 2H), 3.34 (s, 2H), 3.67 (s, 2H), 7.13-7.16 (m, 1H), 7.32-7.36 (m, 1H), 7.60-7.67 (m, 1H), 8.61-8.65 (m, 1H).

LRMS m/z 304 [M+H⁺]

Preparation 64

[Isobutyl-(1-pyridin-2-yl-cyclohexylmethyl)-amino]-acetic Acid Methyl Ester

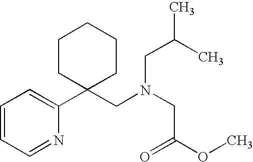

N,N-Diisopropylethylamine (5.5 g, 42 mmol) was added to a solution of the amine from preparation 59 (10 g, 40.6 mmol) and methyl chloroacetate (4.6 g, 42 mmol) in N,N-dimethylacetamide (180 ml), and the reaction heated at 75° C. for 18 hours. The cooled mixture was diluted with ethyl acetate (300 ml), washed with saturated aqueous ammonium chloride solution (100 ml), then saturated sodium bicarbonate solution (100 ml), water (100 ml) and brine (100 ml), dried over magnesium sulphate and concentrated under reduced pressure. The residue was purified by column chromatography on silica gel using ethyl acetate:hexanes (40:60) as eluant to afford the title compound, 6.8 g.

¹H NMR (CDCl₃, 270 MHz): δ 0.74 (d, 6H), 1.23-1.58 (m, 9H), 2.20 (d, 2H), 2.32-2.37 (m, 2H), 2.81 (s, 2H), 2.86 (s, 2H), 3.59 (s, 3H), 7.04-7.09 (m, 1H), 7.34 (m, 1H), 7.55-7.62 (m, 1H), 8.58-8.61 (m, 1H).

LRMS m/z 319 [M+H]⁺

Preparation 65

[Isobutyl-(1-Pyridin-2-yl-cyclohexylmethyl)-amino]-acetic Acid Citrate

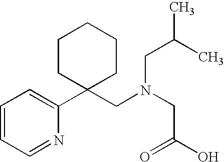

2M Sodium hydroxide solution (30 ml) was added to a solution of the ester from preparation 64 (6.6 g, 20.9 mmol) in methanol (30 ml), and the reaction stirred at room temperature for 12 hours. The mixture was concentrated under reduced pressure and acidified to pH 3 using 1M citric acid, then extracted with dichloromethane (2×100 ml). The combined organic extracts were evaporated under reduced pressure to afford the title compound, 10.1 g.

¹H NMR (CDCl₃, 270 MHz):δ 0.75 (d, 6H), 1.28-1.65 (m, 9H), 2.30-2.35 (m, 2H), 2.51 (d, 2H), 3.17 (s, 2H), 3.55 (s, 2H), 7.25-7.30 (m, 1H), 7.47 (m, 1H), 7.78-7.85 (m, 1H), 8.65 (m, 1H).

LRMS m/z 305 [M+H]⁺

Preparation 66

3-(1-Pyridin-2-yl-cyclohexyl)-acrylic Acid Ethyl Ester

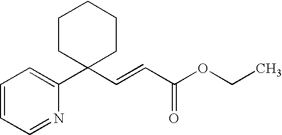

Triethyl phosphonoacetate (7.86 ml, 40 mmol) was added dropwise to a slurry of sodium hydride (2 g, 60% dispersion in mineral oil, 50 mmol) in dry ethylene glycol dimethyl ether (90 ml). Once addition was complete, the mixture was stirred for 1 hour. The aldehyde from preparation 28 (7.5 g, 40 mmol) was added dropwise, so as to maintain the temperature below 30° C., and the reaction stirred for a further 15 minutes. The mixture was suspended in water and extracted with ethyl acetate. The combined organic extracts were dried over magnesium sulphate and evaporated under reduced pressure. The residue was purified by column chromatography on silica gel using a Biotage® column, and an elution gradient of ethyl acetate:heptane (0:100 to 20:80) to afford the title compound, 9.18 g.

LRMS (AP⁺) m/z 260 [M+H]⁺

Preparation 67

3-(1-Pyridin-2-yl-cyclohexyl)-acrylic Acid tert-Butyl Ester

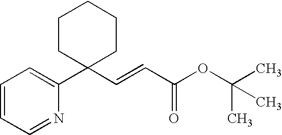

tert-Butyl dimethylphosphonoacetate was added to a suspension of sodium hydride (0.23 g, 60% dispersion in mineral oil, 58.0 mmol) in anhydrous tetrahydrofuran (25 ml) at 0° C. The mixture was stirred at 0° C. for 30 minutes, then a solution of the aldehyde from preparation 28 in anhydrous tetrahydrofuran (25 ml) was added slowly. The reaction mixture was stirred at room temperature for 18 hours. Saturated ammonium chloride solution was added to quench the reaction. The mixture was extracted with ethyl acetate (3×50 ml) and the combined organics were dried over magnesium sulphate, filtered and concentrated to give a yellow oil. The oil was purified by column chromatography on silica gel using hexanes:ethyl acetate (90:10) as eluant to give the title product as a colourless oil, 1.33 g.

LRMS (APCl) m/z 288 (M+H)⁺.

Preparation 68

3-(1-Pyridin-2-yl-cyclohexyl)-propionic Acid Ethyl Ester

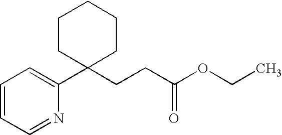

10% Palladium on charcoal, was added to a solution of the alkene from preparation 66 (9.18 g, 35 mmol) in ethanol (200 ml), and the mixture hydrogenated at 50 psi and room temperature for 12 hours. The catalyst was removed by filtration, and the filtrate evaporated under reduced pressure to afford the title compound, 8.83 g.

¹H NMR (CDCl₃, 400 MHz):δ 1.17-1.20 (t, 3H), 1.30-1.60 (m, 8H), 1.96 (m, 4H), 2.24-2.28 (m, 2H), 4.00 (q, 2H), 7.07-8.61 (m, 4H).

LRMS (AP⁺) m/z 262.2 [M+H]⁺

Preparation 69

2-Benzyl-3-(1-pyridin-2-yl-cyclohexyl)-propionic Acid Ethyl Ester

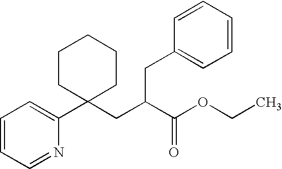

Lithium bis(trimethylsilyl)amide (7.6 ml, 1M in tetrahydrofuran, 7.6 mmol) was added slowly to a cooled (−50° C.) solution of the ester from preparation 67 (2 g, 7.6 mmol) in N,N-dimethylformamide (150 ml), and the mixture stirred at −50° C. for 1 hour. The mixture was cooled to −78° C., benzyl bromide (0.9 ml, 7.6 mmol) added, and the reaction stirred for a further hour, then allowed to warm to room temperature. Water (50 ml) was added, the mixture extracted with ethyl acetate, and the combined organic extracts dried over magnesium sulphate, and evaporated under reduced pressure. The crude product was purified by column chromatography on silica gel using an elution gradient of ethyl acetate:heptane (0:100 to 40:60). The product was further purified by column chromatography on reverse phase silica gel, to afford the title compound, 932 mg.

¹H NMR (CDCl₃, 400 MHz): δ 0.98-1.02 (t, 3H), 1.29-2.24 (m, 12H), 2.39-2.42 (m, 2H), 2.70 (m, 1H), 3.75-3.78 (m, 2H), 6.92-8.66 (m, 9H).

LRMS (AP⁺) m/z 352 [M+H]⁺

Preparation 70

2-(4-Benzyloxy-benzyl)-3-(1-pyridin-2-yl-cyclohexyl)-propionic acid tert-butyl ester

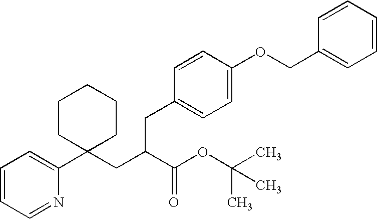

A mixture of the alkene from preparation 67 (1.33 g, 4.61 mmol) and 20% Pd/C (0.1 g) in methanol (50 ml) was shaken under 50 psi of hydrogen at room temperature for 18 hours. The catalyst was removed by filtration and the filtrate was concentrated under reduced pressure. The colourless oil was redissolved in of anhydrous tetrahydrofuran (50 ml) and the solution was concentrated under reduced pressure again to remove any trace of methanol. The residual oil was dissolved in anhydrous tetrahydrofuran (10 ml) and the solution was then added slowly to a solution of lithium diisopropylamide (445 mg, 4.16 mmol) in anhydrous tetrahydrofuran (10 ml) at −78° C. under an Argon atmosphere. The reaction mixture was stirred at −78° C. for 1 hour. A solution of 4-benzyloxybenzyl bromide (1.148 g, 4.14 mmol) in anhydrous tetrahydrofuran (10 ml) was added dropwise to the reaction mixture at −78° C. The reaction was stirred at −78° C. for 45 minutes and then quenched with saturated ammonium chloride (25 ml). After stirring at 0° C. for 15 minutes, the mixture was extracted with ethyl acetate (3×40 ml). The combined organic solutions were dried over magnesium sulphate, filtered and concentrated under reduced pressure. The yellow oil was purified by column chromatography on silica gel eluting with ethyl acetate:hexanes (15:85) to give the desired product as a colorless oil, 1.26 g.

LRMS (APCl) m/z 486.2 (M+H)⁺.

Preparation 71

Lithium 2-Benzyl-3-(1-pyridin-2-yl-cyclohexyl)-propionate

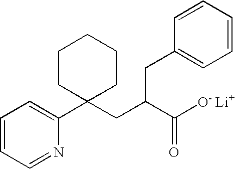

Lithium hydroxide (125.8 mg, 3 mmol) was added to a solution of the ester from preparation 68 (432 mg, 1.23 mmol) in dioxan (15 ml) and water (5 ml), and the reaction stirred at 70° C. for 72 hours. The dioxan was removed under reduced pressure, and the residual aqueous solution was extracted with dichloromethane (3×20 ml). The combined organic extracts were evaporated under reduced pressure to afford the title compound, 466 mg.

¹H NMR (CDCl₃, 400 Hz): δ 1.42-2.18 (m, 10H), 2.20-2.22 (m, 2H), 2.46-2.49 (m, 2H), 2.94 (m, 1H), 5.30 (s, 1H), 7.01-8.49 (m, 9H).

LRMS (AP⁺) m/z 324.3 [M+H]⁺

Preparation 72

2-(4-Benzyloxy-benzyl)-3-(1-pyridin-2-yl-cyclohexyl)-propionic Acid

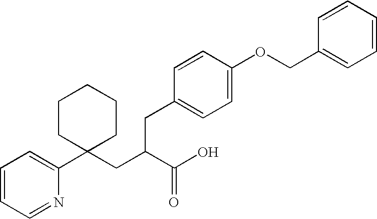

A solution of the ester from preparation 70 (1.12 g, 2.30 mmol) and trifluoroacetic acid (4 ml) in dichloromethane (15 ml) was stirred at room temperature for 18 hours. The reaction mixture was concentrated under reduced pressure and the residue was redissolved in chloroform (40 mL) and the solution was again concentrated under reduced pressure. The residue was dissolved in ethyl acetate (50 ml), then saturated ammonium chloride solution (30 ml) and saturated sodium bicarbonate solution (10 ml) were added. The mixture was extracted with ethyl acetate (2×50 ml). The combined organic layers were dried over magnesium sulphate, filtered and evaporated under reduced pressure to give the title compound, 0.99 g.

LRMS (APCl) m/z 430 (M+H)⁺.

Preparation 73

2-(4-Benzyloxy-benzyl)-N-(2,6-diisopropyl-phenyl)-3-(1-pyridin-2-yl-cyclohexyl)-propioniamide

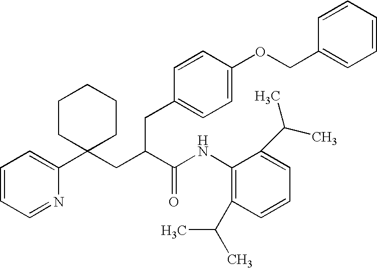

A mixture of the acid from preparation 72 (0.316 g, 0.74 mmol), 2,6-diisopropylaniline (0.15 ml, 0.80 mmol), N,N-diisopropylcarbodiimide (0.17 ml, 1.09 mmol) and a catalytic amount of 4-dimethylaminopyridine in tetrahydrofuran (5 ml) was stirred at room temperature for 18 hours and then at 60° C. for 1 hour. After cooling to room temperature, saturated sodium chloride solution was added and the mixture was extracted with ethyl acetate (2×30 ml). The combined organic layers were dried over magnesium sulphate, filtered and concentrated. The residue was purified by column chromatography on silica gel using ethyl acetate:hexanes (20:80) to give the desired product as a white solid, 0.286 g.

LRMS (APCl) m/z 589.3 (M+H)⁺.

Preparation 74

N′-(Acetyloxy)-3-[([(2,6-diisopropyl-anilino)carbonyl]{8 1-(2-pyridinyl)cyclohexyl]methyl}amino)methyl]benzenecarboximidamide

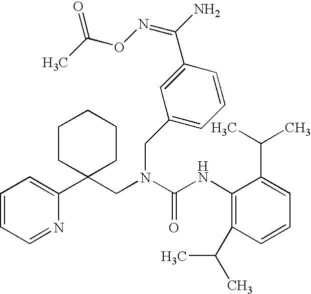

Hydroxylamine hydrochloride (600 mg, 8.6 mmol) and potassium carbonate (1 g, 7.2 mmol) were added to a solution of the nitrile from example 130 (1 g, 2.0 mmol) in methanol (30 ml) and water (5 ml), and the reaction heated under reflux for 2 hours. The mixture was cooled, diluted with water (100 ml), and the products extracted using ethyl acetate (2×100 ml). The combined organic solutions were dried over magnesium sulphate and concentrated under reduced pressure. The product was purified by column chromatography on silica gel using methanol:dichloromethane (5:95) as eluant to afford a colourless oil, 720 mg.

A solution of this compound in acetic acid (20 ml) and acetic anhydride (1 ml), 10.6 mmol) was stirred at room temperature for 20 minutes. The mixture was concentrated under reduced pressure and the residue purified by column chromatography on silica gel using an elution gradient of methanol:dichloromethane (0:100 to 5:95) to afford the title compound as a white foam, 510 mg.

LRMS (AP⁺) m/z 584 [M+H]⁺

Preparation 75

1-Benzothiazol-2-yl-cyclohexanecarbonitrile

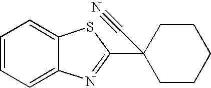

Sodium hydride (60% in mineral oil, 0.3 g, 7.5 mmol) was dissolved in N,N-dimethylformamide (30 ml) and was cooled to 0° C. A solution of 1,5-dibrompentane (0.44 ml, 3.2 mmol) and benzothiazole-2-acetonitrile (0.5 g, 2.9 mmol) in N,N-dimethylformamide (10 ml) was added dropwise over 15 minutes. The reaction was warmed to room temperature and was stirred for 40 minutes. The reaction mixture was diluted with 2N hydrochloric acid (100 ml) and the aqueous solution was extracted with ethyl acetate (3×70 ml). The combined organic extracts were dried over magnesium sulphate and evaporated under reduced pressure. The residue was purified by chromatography on silica gel using ethyl acetate in heptane as eluant (10:90) to give the title compound as a colourless oil (0.61 g).

LRMS (APCl) m/z 503 [M+H]⁺

Preparation 76

1-(1-Methyl-1H-pyrrol-2-yl)-cyclohexanecarbonitrile

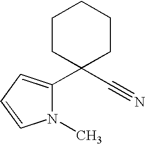

Sodium hydride (60% in mineral oil, 2.64 g, 66 mmol) was washed with heptane (3×30 ml) under a nitrogen atmosphere and suspended in dimethylsulphoxide (100 ml). A solution of (1-methyl-1H-pyrrol-2-yl)-acetonitrile (3.6 g, 30 mmol)(See reference J. Org. Chem. 1977, 42(6), 1096) and 1,5-dibromopentane (4.5 ml, 33 mmol) in diethyl ether (30 ml) was added dropwise over 1 hour maintaining the reaction temperature below 25° C. and the mixture was then stirred at room temperature for 16 hours. Propan-2-ol (5 ml) and then water (200 ml) were added and the aqueous mixture was extracted with ethyl acetate (3×100 ml). The combined organic layers were dried over magnesium sulphate and evaporated under reduced pressure. The residue was purified by chromatography on silica gel using ethyl acetate in heptane as eluant (8:92) to give the title compound as an oil (3.71 g).

¹H NMR (400 MHz, CDCl₃): δ 1.26 (m, 1H), 1.75 (m, 7H), 2.36 (m, 2H), 3.83 (s, 3H), 6.01 (s, 1H), 6.06 (s, 1H), 6.62 (s, 1H)

Preparations 77 to 79

The compounds of the following tabulated preparations of the general formula

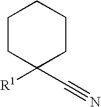

were prepared by a similar method to that from preparation 76 using the appropriate nitrile starting material.

| Preparation | R¹ | Data |
|---|---|---|
| 77 | 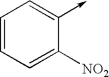 | LRMS (APCI) m/z 201 [M + H]⁺ |
| 78 | 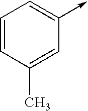 | ¹H NMR(400MHz, DMSO-d₆):δ1.30 (m, 1H), 1.72(m, 7H), 2.03(m, 2H), 2.34(s, 3H), 7.16(m, 1H), 7.32 (m, 3H) |
| 79 | 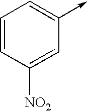 | ¹H NMR(400MHz, DMSO-d₆);δ1.33 (m, 1H), 1.78(m, 7H), 2.13(m, 2H), 7.76(m, 1H), 8.05(m, 1H), 8.23(m, 1H), 8.34(m, 1H) |

Preparation 80

1-(3H-Imidazol-4-yl)cyclohexanecarbonitrile

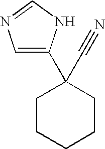

A solution of 1-(3-trityl-3H-imidazol-4-yl)-cyclohexanecarbonitrile (WO 9807718, page 98) (8.98 g, 21.5 mmol) in methanol (150 ml) and 2N hydrochloric acid (25 ml) was heated under reflux for 4 hours, and then the solution was concentrated under reduced pressure. The residue was dissolved in water, washed with ether, and the aqueous layer basified using saturated sodium bicarbonate solution. This aqueous mixture was extracted with ethyl acetate, and the combined organic extracts dried over magnesium sulphate and evaporated under reduced pressure to give the title compound as a yellow solid, 3.58 g.

Preparation 81

1-(3-Bromomethyl-phenyl)-cyclohexanecarbonitrile

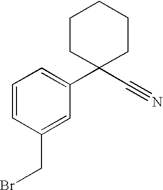

Benzoyl peroxide (75 mg, 0.3 mmol) in carbon tetrachloride (10 ml) was added to a solution of the compound of preparation 78 (2.78 g, 14 mmol) and N-bromosuccinimide (2.74 g, 15.4 mmol) in carbon tetrachloride (40 ml) under a nitrogen atmosphere. The mixture was heated under reflux for 4.5 hours, then cooled to room temperature and was stirred for a further 20 hours. The reaction mixture was filtered and the filter cake was washed with carbon tetrachloride (2×10 ml). The combined filtrates were evaporated under reduced pressure. The residue was purified by chromatography on silica gel using ethyl acetate in heptane as eluant (gradient from 0:100 to 40:60) to give the title compound (3.37 g)

¹H NMR (400 MHz, CDCl₃): δ 1.82 (m, 8H, 2.15 (m, 2H), 4.50 (s, 2H), 7.42 (m, 4H)

Preparation 82

1-(3-Dimethylaminomethyl-phenyl)-cyclohexanecarbonitrile

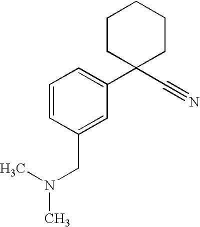

Dimethylamine (2M in tetrahydrofuran, 24 ml, 48 mmol) was added to the bromo compound of preparation 81 (3.34 g, 12 mmol) in dichloromethane (100 ml) under a nitrogen atmosphere and the mixture was stirred at room temperature for 20 hours. 1N Sodium hydroxide solution (50 ml) was added and the mixture was stirred for 10 minutes. The layers were separated and the aqueous phase was extracted with dichloromethane (25 ml), the combined organic solutions were dried over potassium carbonate and evaporated under reduced pressure. The residue was purified by chromatography on silica gel using ethyl acetate in heptane as eluant (gradient from 0:100 to 100:00) to give the title compound as a yellow oil (1.86 g).

¹H NMR (400 MHz, CDCl₃): δ 1.76 (m, 8H), 2.16 (m, 2H), 2.24 (d, 6H), 3.44 (s, 2H), 7.25 (m, 1H), 7.34 (m, 1H), 7.40 (d, 1H), 7.42 (s, 1H)

Preparation 83

[1-(3-Dimethylaminomethyl-phenyl)-cyclohexyl]-methylamine Dihydrochloride

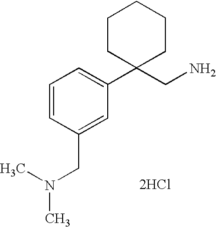

The nitrile from preparation 82 (1.76 g, 7.3 mmol) was dissolved in methanolic ammonia (50 ml) and was added to Raney® Nickel (1.76 g) that had been washed with water (×2) and then suspended in ethanol (20 ml). The reaction mixture was hydrogenated at 30° C. for 16 hours and then filtered through kieselguhr. The solvent was evaporated under reduced pressure and the material obtained was dissolved in dichloromethane and hydrogen chloride (4M in 1,4-dioxane) was added. The solvent was evaporated under reduced pressure to give the title compound as a white solid.

LRMS (APCl) m/z 247 [M+H]⁺

Preparation 84

1-(1H-Benzimidazol-2-yl)-cyclohexanecarbonitrile

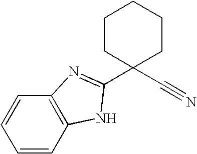

Sodium hydride (60% in mineral oil, 0.75 g, 4.8 mmol) was added to benzimidazole-2-acetonitrile (0.7 g, 4.4 mmol) in N,N-dimethylformamide (30 ml) at 0° C. and the mixture was stirred for 20 minutes. 1,5-Dibromopentane (0.65 ml, 4.8 mmol) was added and the mixture was stirred at room temperature for 19 hours. Water (100 ml) was added and the aqueous mixture extracted with ethyl acetate (2×100 ml). The combined organic solutions were dried over magnesium sulphate and evaporated under reduced pressure. The residue was purified by chromatography on silica gel using ethyl acetate in heptane as eluant (gradient from 0:100 to 50:50). The material isolated was triturated with heptane to give the title compound as a white solid (0.7 g).

LRMS (APCl) m/z 226 [M+H]⁺

Preparation 85

1-(1-Methyl-1H-benzimidazol-2-yl)-cyclohexanecarbonitrile

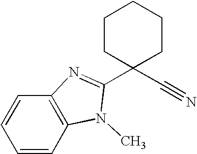

Sodium hydride (60% in mineral oil, 0.1 g, 2.5 mmol) was added to the benzimidazole of preparation 84 (0.35 g, 1.56 mmol) in N,N-dimethylformamide (20 ml) and the mixture was stirred for 10 minutes. Iodomethane (0.3 g, 2.1 mmol) was added and the mixture was stirred for 1 hour and then water (150 ml) was added. The aqueous mixture was extracted with ethyl acetate (2×100 ml) and the combined organic solutions were dried over magnesium sulphate and evaporated under reduced pressure. The residue was purified by chromatography on silica gel using ethyl acetate in heptane as eluant (gradient from 0:100 to 40:60) to give the title compound as a yellow oil (0.32 g).

LRMS (APCl) m/z 240 [M+H]⁺

Preparation 86

1-(3-Amino-phenyl)-cyclohexanecarbonitrile

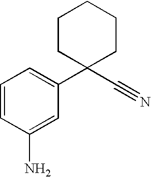

10% Palladium on active carbon (100 mg) was added to a suspension of the nitro compound of preparation 79 (5 g, 22 mmol) in methanol (50 ml) and the mixture was hydrogenated at 30° C. for 4 hours. The reaction mixture was filtered and the solvent was evaporated under reduced pressure to give the title compound as a yellow oil (4.16 g).

¹H NMR (400 MHz, DMSO-d₆): δ 1.28 (m, 1H), 1.69 (m, 7H), 1.98 (m, 2H), 5.18 (m, 2H), 6.48 (m, 1H), 6.60 (m, 1H), 6.71 (m, 1H), 7.03 (m, 1H)

Preparation 87

1-(3-Dimethylamino-phenyl)-cyclohexanecarbonitrile

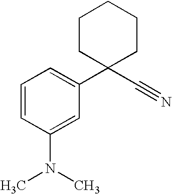

2,6-Lutidine (4.45 g, 42 mmol) and iodomethane (2.58 ml, 42 mmol) were added to the aniline from preparation 86 (4.16 g, 21 mmol) and the mixture was stirred at room temperature for 3 hours. Additional iodomethane (2.58 ml, 42 mmol) was added and the mixture was stirred for a further 72 hours. A further quantity of iodomethane (2.58 ml, 42 mmol) was added and the mixture was stirred for a further 16 hours. The solvent was evaporated under reduced pressure and the residue was purified by chromatography on silica gel using ethyl acetate in heptane as eluant (gradient from 0:100 to 8:92) to give the title compound as an orange oil (1.86 g).

¹H NMR (400 MHz, DMSO-d₆): δ 1.30 (m, 1H), 1.72 (m, 7H), 2.04 (m, 2H), 2.91 (s, 6H), 6.68 (m, 1H), 6.78 (m, 2H), 7.20 (m, 1H)

Preparation 88

[3-(1-Aminomethyl-cyclohexyl)-phenyl]-dimethyl-amine

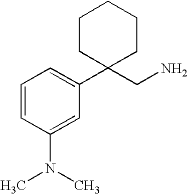

The nitrile from preparation 87 (1.85 g, 8.1 mmol) was dissolved in ethanolic ammonia (40 ml) and was added to Raney® Nickel (1.85 g) suspended in ethanol (20 ml). The reaction mixture was hydrogenated at 30° C. for 16 hours and then filtered. The solvent was evaporated under reduced pressure to give the title compound as a yellow oil (1.78 g).

¹H NMR (400 MHz, CDCl₃): δ 1.20 (s, 2H), 1.43 (m, 8H), 2.12 (m, 2H), 2.69 (s, 2H), 2.95 (s, 6H), 6.61 (m, 1H), 6.72 (m, 2H), 7.21 (m, 1H)

Preparation 89

(1-Benzothiazol-2-yl-cyclohexyl)-methylamine

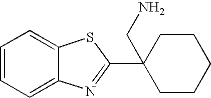

Lithium aluminium hydride (1M solution in diethyl ether, 2.5 ml, 2.5 mmol) was added to a solution of the nitrile of preparation 75 (0.6 g, 2.5 mmol) in diethyl ether (40 ml) and the mixture was stirred for 30 minutes. Saturated sodium carbonate solution (50 ml) was added and the organic layer was separated. The aqueous layer was extracted with ethyl acetate (100 ml) and the combined organic solutions were dried over magnesium sulphate and evaporated under reduced pressure The residue was purified by chromatography on silica gel using methanol in dichloromethane as eluant (gradient from 0:100 to 5:95) to give the title compound as a yellow oil (0.42 g).

LRMS (APCl) m/z 247 [M+H]⁺

Preparation 90

(1-Benzo[b]thiophen-3-yl-cyclohexyl)-methylamine

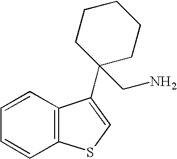

Sodium hydride (60% in mineral oil, 0.3 g, 7.5 mmol) was dissolved in N,N-dimethylformamide (30 ml) and was cooled to 0° C. A solution of 1,5-dibrompentane (0.44 ml, 3.2 mmol) and benzo[b]thiophen-3-yl-acetonitrile (0.5 g, 2.9 mmol) in N,N-dimethylformamide (10 ml) was added dropwise over 15 minutes. The reaction mixture was stirred at 0° C. for 30 minutes, then warmed to room temperature and was stirred for 2 hours. The reaction mixture was diluted with 2N hydrochloric acid (100 ml) and the aqueous solution was extracted with ethyl acetate (3×70 ml). The combined organic extracts were dried over magnesium sulphate and evaporated under reduced pressure. The residue was purified by chromatography on silica gel using ethyl acetate in heptane as eluant (gradient from 0:100 to 30:70) to give a yellow oil.

The material obtained was dissolved in tetrahydrofuran (30 ml) and lithium aluminium hydride (1M solution in diethyl ether, 3 ml, 3 mmol) was added. The reaction mixture was stirred at room temperature for 1 hour and then water (50 ml) was added. The phases were separated and the aqueous phase was extracted with ethyl acetate (50 ml). The combined organic phases were dried over magnesium sulphate and evaporated under reduced pressure. The residue was purified by chromatography on silica gel using methanol in ethyl acetate as eluant (0:100 to 10:90) to give the title compound as a yellow oil (0.48 g).

LRMS (APCl) m/z 503 [M+H]⁺

Preparation 91

Ethyl 1-(1-methyl-1H-tetrazol-5-yl)-cyclohexanecarboxylate

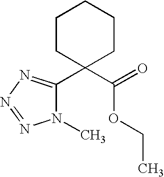

Sodium carbonate was added to a mixture of 2-ethoxycarbonyl-but-2-enedioic acid diethyl ester (21.6 g, 100 mmol) and methyl hydroxylamine hydrochloride (8.4 g, 55 mmol) and the mixture was stirred at room temperature for 16 hours. The reaction mixture was filtered and the filtrate was heated at 130° C. using a distillation head for removal of ethanol, for 1 hour. The reaction mixture was cooled to room temperature and the residue was recrystallised from toluene.

The material obtained was dissolved in water (70 ml) and sodium azide (6.46 g, 99 mmol) was added. The reaction mixture was stirred at room temperature for 3 days and then was extracted into dichloromethane (3×100 ml). The combined organic layers were dried over magnesium sulphate and evaporated under reduced pressure.

The material isolated was dissolved in dimethylsulphoxide (50 ml) and diethyl ether (50 ml) containing 1,5-dibromopentane (4 g, 17.5 mmol). The solution was added dropwise to a suspension of sodium hydride (60% in mineral oil, 1.4 g, 35 mmol, freshly washed with heptane) in dimethylsulphoxide under a nitrogen atmosphere at a rate that maintained the reaction temperature below 30° C. The reaction mixture was stirred at room temperature for 24 hours and then was added to water (2000 ml). The aqueous solution was extracted with diethyl ether (3×200 ml) and the combined organic layers were dried over magnesium sulphate and evaporated under reduced pressure. The residue was purified by chromatography on silica gel using ethyl acetate in heptane as eluant, to provide the title compound, 2.56 g.

¹H NMR (400 MHz, CDCl₃): δ 1.22 (t, 3H), 1.48-2.70 (m, 6H), 2.20-2.34 (m, 4H), 4.00 (s, 3H), 4.18 (q, 2H).

Preparation 92

1-(1-Methyl-1H-tetrazol-5-yl)-cyclohexanecarboxylic acid

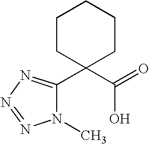

The ester from preparation 92 (2.02 g, 85 mmol) was dissolved in 1,4-dioxane (50 ml) and water (25 ml) and lithium hydroxide hydrate (0.71 g, 17 mmol) was added. The mixture was stirred at room temperature for 16 hours and 1N hydrochloric acid (17 ml, 17 mmol) was added. The mixture was concentrated under reduced pressure and the solid formed was isolated by filtration to give the title compound (1.71 g).

¹H NMR (400 MHz, DMSO-d₆): δ 1.46 (m, 6H), 2.04 (m, 4H), 4.00 (s, 3H), 13.43 (s, 1H)

Preparation 93

1-(Benzyloxycarbonylamino-methyl)-cyclohexanecarboxylic acid ethyl ester

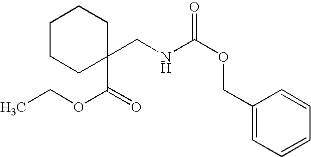

Benzyl chloroformate (1.2 g, 7.1 mmol) and potassium carbonate (1 g, 7.2 mmol) were added to a solution of the amine of preparation 26 (1 g, 6.4 mmol) in 1,4-dioxane (30 ml) containing water (5 ml). The mixture was stirred for 30 minutes and then was diluted with ethyl acetate. The reaction mixture was washed with 2N hydrochloric acid (2×100 ml), dried over magnesium sulphate and evaporated under reduced pressure. The residue was purified by chromatography on silica gel using ethyl acetate in heptane as eluant (30:70) to give the title compound as a colourless oil (1.7 g).

LRMS (APCl) m/z 320 [M+H]⁺

Preparation 94

1-(tert-Butoxycarbonylamino-methyl)-cyclohexanecarboxylic acid ethyl ester

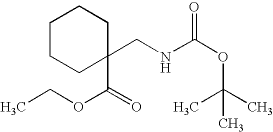

A solution of di-tert-butyl dicarbonate (13.08 g, 60 mmol) in dioxan (20 ml) was added to a solution of the amine from preparation 26 (10.0 g, 54.05 mmol) and sodium bicarbonate (11.35 g) in dioxan (80 ml) and water (50 ml), and the reaction stirred at room temperature for 5 hours. The mixture was diluted with ethyl acetate, washed with water, 10% hydrochloric acid, then brine, dried over magnesium sulphate and evaporated under reduced pressure to give the title compound as a brown oil, 10.7 g.

¹H NMR (400 MHz, CDCl₃): δ 1.28 (t, 3H), 1.29-1.70 (m, 8H), 1.43 (s, 9H), 2.00 (m, 2H), 3.27 (d, 2H), 4.16 (q, 2H), 4.76 (s, 1H).

Preparation 95

1-(Benzyloxycarbonylamino-methyl)-cyclohexanecarboxylic acid

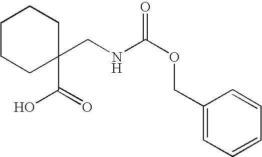

The ester of preparation 93 (1.7 g, 5.3 mmol) was dissolved in tetrahydrofuran (40 ml), methanol (20 ml) and water (10 ml) and lithium hydroxide hydrate (0.5 g, 12 mmol) was added. The mixture was stirred for 12 hours and then evaporated under reduced pressure. The residue was partitioned between ethyl acetate (150 ml) and 2N hydrochloric acid (100 ml). The organic layer was separated, dried over magnesium sulphate and evaporated under reduced pressure to give the title compound as a colourless oil (1.4 g).

LRMS (APCl) m/z 292 [M+H]⁺

Preparation 96

1-(tert-Butoxycarbonylamino-methyl)-cyclohexanecarboxylic acid

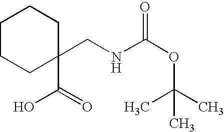

A mixture of the ester from preparation 94 (15.4 g, 54 mmol) and lithium hydroxide (4.54 g, 108 mmol) in tetrahydrofuran (40 ml) and water (10 ml) was stirred at room temperature for 20 hours, then under reflux for a further 3 days. The mixture was concentrated under reduced pressure and the residue acidifed using 2N hydrochloric acid to pH 2, and extracted with ethyl acetate. The combined organic extracts were washed with brine, dried over magnesium sulphate and evaporated under reduced pressure. The product was recrystallised from isopropyl alcohol to afford the title compound as a white powder, 7.1 g.

¹H NMR (400 MHz, CDCl₃): δ 1.05-1.30 (m, 5H), 1.36 (s, 9H), 1.50 (m, 3H), 1.84 (m, 2H), 3.02 (d, 2H), 6.68 (s, 1H), 12.10 (s, 1H).

Preparation 97

(1-Dimethylcarbamoyl-cyclohexylmethyl)-carbamic acid benzyl ester

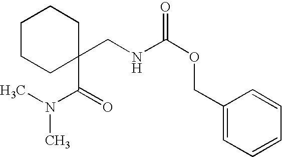

The carboxylic acid from preparation 95 (1.4 g, 4.8 mmol) was dissolved in N,N-dimethylformamide (30 ml) and O-benzotriazol-1-yl-N,N,N′,N′,-tetramethyluronium hexafluorophosphate (2 g, 5.3 mmol), triethylamine (1 ml, 7 mmol) and dimethylamine (2M in tetrahydrofuran, 3 ml, 6 mmol) were added. The reaction mixture was stirred at room temperature for 1 hour and then partitioned between ethyl acetate (50 ml) and 2N hydrochloric acid (100 ml). The organic layer was separated, dried over magnesium sulphate and evaporated under reduced pressure. The residue was purified by chromatography on silica gel using methanol in dichloromethane as eluant (5:95) to give the title compound (1.45 g).

LRMS (APCl) m/z 319 [M+H]⁺

Preparation 98

1-[(4-Hydroxy-benzylamino)-methyl]-cyclohexanecarboxylic acid dimethyl amide

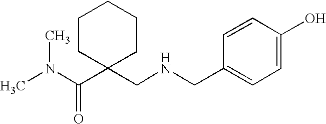

The amide from preparation 97 was dissolved in methanol (40 ml) and ammonium formate (0.5 g, 7.9 mmol) and 5% palladium on active carbon (50 mg) were added. The mixture was heated under reflux for 30 minutes and then cooled to room temperature. The mixture was filtered and evaporated under reduced pressure. The material isolated was dissolved in dichloromethane (50 ml) and 4-hydroxy benzaldehyde (0.26 g, 2.1 mmol) and sodium triacetoxyborohydride (1.5 g, 7.5 mmol) were added. The mixture was stirred at room temperature for 30 minutes and then sodium carbonate solution (100 ml) was added. The aqueous mixture was extracted with ethyl acetate (2×100 ml) and the combined organic layers were dried over magnesium sulphate and evaporated under reduced pressure. The residue was purified by chromatography on silica gel using ethyl acetate in heptane (30:70) then changing to methanol in dichloromethane (10:90) as eluant to give the title compound (0.41 g).

LRMS (APCl) m/z 319 [M+H]⁺

Preparation 99

[1-(Morpholine-4-carbonyl)-cyclohexylmethyl]-carbamic acid tert-butyl ester

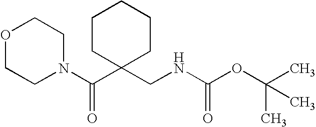

O-benzotriazol-1-yl-N,N,N′,N′,-tetramethyluronium hexafluorophosphate (7.94 g, 31 mmol) in N,N-dimethylformamide (10 ml) and N,N-diisopropylethylamine (10.7 ml, 62 mmol) were added to the carboxylic acid of preparation 96 (7 g, 30.8 mmol) in N,N-dimethylformamide (15 ml) and the mixture was stirred at room temperature for 1 hour. Morpholine (2.68 ml, 62 mmol) in N,N-dimethylformamide (10 ml) was added and the mixture was stirred for 18 hours at 50° C. The reaction mixture was cooled to room temperature and evaporated under reduced pressure. The residue was dissolved in ethyl acetate and was washed with sodium carbonate solution (3×30 ml), 1M hydrochloric acid (3×30 ml) and brine (3×30 ml). The organic layer was separated and dried over sodium sulphate then evaporated under reduced pressure. The oil obtained crystallised on standing and was recrystallised from ethyl acetate/hexane to give the title compound (4 g).

LCMS: m/z ES⁺ 349 [M+Na]⁺

Preparation 100

(1-Aminomethyl-cyclohexyl)-morpholin-4-yl-methanone Hydrochloride

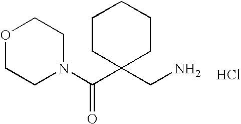

Hydrogen chloride (4M in diethyl ether, 30 ml) was added to the protected amine from preparation 99 (6.3 g, 19 mmol) and the mixture was stirred at room temperature for 16 hours. The solvent was evaporated under reduced pressure and the residue was re-suspended in diethyl ether. The solid formed was isolated by filtration to give the title compound as a white solid (6.0 g).

LCMS: m/z ES⁺ 227 [M+H]⁺

Preparation 101

[1-(1-Methyl-1H-pyrrol-2-yl)-cyclohexyl]-methylamine

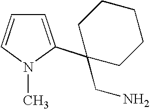

Borane-methyl sulphide complex (2M in toluene, 6.5 ml, 13 mmol) was added to the nitrile of preparation 76 (2.26 g, 12 mmol) in toluene (100 ml) under reflux. The reaction mixture was heated under reflux under a nitrogen atmosphere for 2 hours and then was cooled to room temperature. Methanol (100 ml) was added dropwise and the pH of the reaction mixture was adjusted to 2 by addition of 4M hydrochloric acid. The mixture was heated under reflux for 1 hour, then was cooled to room temperature and evaporated under reduced pressure. Methanol (100 ml) was added and the mixture was evaporated under reduced pressure. The residue was partitioned between 15% sodium hydroxide solution and dichloromethane and the aqueous solution was extracted with dichloromethane (×2). The combined organic layers were dried over potassium carbonate solution and evaporated under reduced pressure. The residue was purified by chromatography on silica gel using methanol and ammonium hydroxide in ethyl acetate as eluant (10:1:90) to give the title compound (1.6 g).

LRMS (APCl) m/z 193 [M+H]⁺

Preparation 102

[1-(2-Nitro-phenyl)-cyclohexyl]-methylamine Hydrochloride

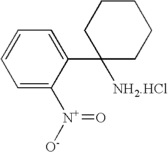

Borane-methyl sulphide complex (2M in toluene, 11 ml, 22 mmol) was added dropwise over 30 minutes to the nitrile compound of preparation 77 (4.6 g, 20 mmol) under a nitrogen atmosphere. The mixture was heated under reflux for 2 hours and then was cooled to room temperature. Methanol (150 ml) was added dropwise and the reaction mixture was adjusted to pH 2 by addition of 4M hydrochloric acid. The mixture was heated under reflux for 1 hour and then was cooled to room temperature and evaporated under reduced pressure. Methanol (100 ml) was added and the mixture was evaporated under reduced pressure. The residue was partitioned between 15% sodium hydroxide solution and dichloromethane and the aqueous solution was extracted with dichloromethane (2×100 ml). The combined organic layers were dried over potassium carbonate solution and evaporated under reduced pressure. The residue was purified by chromatography on silica gel using methanol and ammonium hydroxide in ethyl acetate as eluant (10:1:90) to give an oil (2.8 g).

This product (0.23 g) was dissolved in dichloromethane and hydrogen chloride (4M in 1,4-dioxane) was added. The solvent was evaporated under reduced pressure and the residue was triturated with diethyl ether to give the title compound (0.24 g).

LRMS (APCl) m/z 235 [M+H]⁺

Preparation 103

[1-(2,6-Difluoro-phenyl)-cyclohexyl]-methylamine hydrochloride

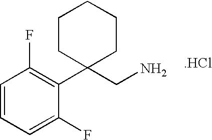

A solution of 2,6-difluorophenylacetonitrile (2.30 g, 15 mmol) and 1,5 dibromopentane (3.79 g, 16.5 mmol) in ether (30 ml), was added dropwise to a suspension of sodium hydride (1.32 g, 60% dispersion in mineral oil, 33 mmol), previously washed with heptane, in DMSO (50 ml) in a cooling bath, so as to maintain the temperature at 25° C. Once addition was complete, the reaction was stirred vigorously for 18 hours. The reaction was quenched by the addition of isopropanol (5 ml), then carefully diluted with water (150 ml). The mixture was extracted with ethyl acetate (3×100 ml), and the combined organic extracts were washed with brine, dried over magnesium sulphate and evaporated under reduced pressure. The residue was purified by column chromatography on silica gel using ethyl acetate in heptane (8:92), to give a solid, 2.58 g.

Borane-methyl sulphide complex (2M in toluene, 5.5 ml, 11 mmol) was added dropwise over 30 minutes to a solution of this solid (2.2 g, 10 mmol) in toluene (50 ml) under a nitrogen atmosphere. The mixture was heated under reflux for 4 hours and then was cooled to room temperature. Methanol (50 ml) was added dropwise and the reaction mixture was adjusted to pH 2 by addition of 4M hydrochloric acid in dioxan. The mixture was heated under reflux for 30 minutes and then was cooled to room temperature and evaporated under reduced pressure. Methanol (50 ml) was added and the mixture was evaporated under reduced pressure. The residue was partitioned between 15% sodium hydroxide solution and dichloromethane the layers separated and the aqueous phase extracted with dichloromethane. The combined organic solutions were dried over potassium carbonate and evaporated under reduced pressure. The crude product was purified by column chromatography on silica gel using ethyl acetate as eluant to afford the title compound, 1.35 g.

LRMS (APCl) m/z 226 [M+H]⁺

Preparation 104

4-({[1-(1-Methyl-1H-imidazol-4-yl)-cyclohexylmethyl]-amino}-methyl)-phenol

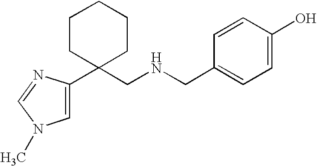

The nitrile from preparation 80 (1 g, 5.7 mmol) in tetrahydrofuran (50 ml) was added to sodium hydride (60% in mineral oil, 0.25 g, 6.3 mmol) and then iodomethane (1 g, 7.2 mmol) was added. The reaction mixture was stirred at room temperature for 1 hour and saturated sodium carbonate solution (100 ml) was added. The aqueous solution was extracted with ethyl acetate (2×100 ml) and the combined organic layers were dried over magnesium sulphate and evaporated under reduced pressure. The residue was purified by chromatography on silica gel using methanol in dichloromethane as eluant (5:95).

The material obtained was dissolved in tetrahydrofuran (50 ml) and lithium aluminium hydride (1M in tetrahydrofuran, 5 ml, 5 mmol) was added. The mixture was stirred at room temperature for 30 minutes and then methanol (10 ml) was added. The solvent was evaporated under reduced pressure and the residue was dissolved in dichloromethane (100 ml). 4-Hydroxybenzaldehyde (0.3 g, 2.5 mmol) and sodium triacetoxyborohydride (1 g, 4.7 mmol) were added. The reaction mixture was stirred at room temperature for 2 hours and then heated to 80° C. for 1 hour. The mixture was cooled to room temperature and water (200 ml) was added. The aqueous solution was extracted with ethyl acetate (2×100 ml) and the combined organic solutions were dried over magnesium sulphate and evaporated under reduced pressure to give the title compound (0.42 g).

LRMS (APCl) m/z 300 [M+H]⁺

Preparation 105

4-({[1-(1-Methyl-1H-benzimidazol-2-yl)-cyclohexylmethyl]-amino}-methyl)phenol

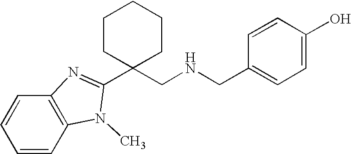

The nitrile from preparation 85 (0.32 g, 1.3 mmol) was dissolved in tetrahydrofuran (30 ml) and lithium aluminium hydride (1M solution in diethyl ether, 1.5 ml, 1.5 mmol) was added. The reaction mixture was stirred at room temperature for 1 hour and then water (50 ml) was added. The phases were separated and the aqueous phase was extracted with ethyl acetate (2×100 ml). The combined organic solutions were dried over magnesium sulphate and evaporated under reduced pressure.

The residue was dissolved in dichloromethane (60 ml) and 4-hydroxybenzaldehyde (0.2 g, 1.6 mmol) and sodium triacetoxyborohydride (1.5 g, 7.5 mmol) were added. The reaction mixture was stirred at room temperature for 2 hours and then water (200 ml) was added. The phases were separated and the aqueous solution was extracted with ethyl acetate (2×150 ml). The combined organic solutions were dried over magnesium sulphate and evaporated under reduced pressure. The residue was purified by chromatography on silica gel using methanol in dichloromethane as eluant (gradient from 0:100 to 10:90) to give the title compound as a yellow oil (0.27 g).

LRMS (APCl) m/z 350 [M+H]⁺

Preparation 106

4-({[1-(1-Methyl-1H-pyrrol-2-yl)-cyclohexylmethyl]-amino}-methyl)-phenol

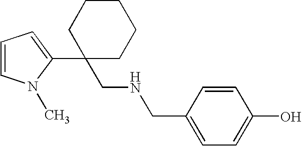

4-Hydroxybenzaldehyde (40 mg, 0.3 mmol) was added to the amine of preparation 101 (65 mg, 0.3 mmol) in dichloromethane (20 ml) and then sodium triacetoxyborohydride (300 mg, 1.5 mmol) was added. The reaction mixture was stirred at room temperature for 4 hours and then sodium carbonate solution (50 ml) was added. The mixture was extracted with ethyl acetate (2×100 ml) and the combined organic solutions were dried over magnesium sulphate and evaporated under reduced pressure. The residue was purified by chromatography on silica gel using ethyl acetate in dichloromethane as eluant (gradient from 0:100 to 90:10) to give the title compound as a yellow oil (82 mg).

LRMS (APCl) m/z 299 [M+H]⁺

Preparations 107 to 115:

The compounds of the following tabulated preparations of the general formula:

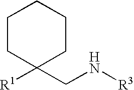

were prepared by a similar method to that of preparation 107 using the appropriate amine and aldehyde.

| Prep. | | | |
|---|---|---|---|
| no | R1 | R3 | Data |
| 107 | 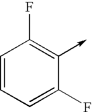 | 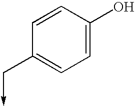 | LRMS (APCI) m/z 332 [M + H]⁺ |
| 108 | 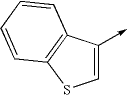 | 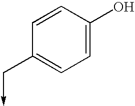 | LRMS (APCI) m/z 352 [M + H]⁺ |
| 109 | 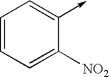 | 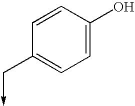 | LRMS (APCI) m/z 341 [M + H]⁺ |
| 110 | 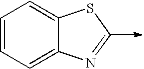 | 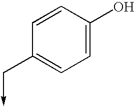 | LRMS (APCI) m/z 353 [M + H]⁺ |
| 111 | 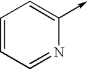 | 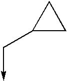 | LRMS (APCI) m/z 245 [M + H]⁺ |
| 112 | 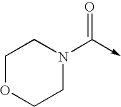 | 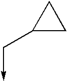 | LRMS (APCI) m/z 281 [M + H]⁺ |
| 113 | 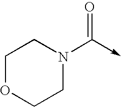 | 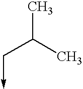 | LRMS (APCI) m/z 283 [M + H]⁺ |
| 114 | 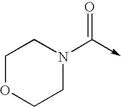 | 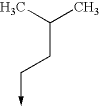 | LRMS (APCI) m/z 297 [M + H]⁺ |
| 115 | 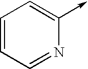 | 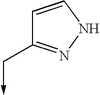 | LRMS (APCI) m/z 271 [M + H]⁺ |

A. The reaction mixture was worked up by addition of 0.5M sodium hydroxide solution instead of sodium carbonate solution.

Preparation 116

4-({[1-(3-Dimethylamino-phenyl)-cyclohexylmethyl]-amino}-methyl)-phenol

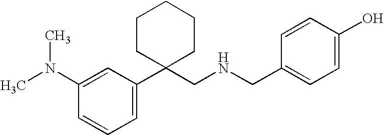

4-Hydroxybenzaldehyde (40 mg, 0.3 mmol) was added to the amine of preparation 88 (80 mg, 0.3 mmol) in dichloromethane (2 ml) and then sodium triacetoxyborohydride (300 mg, 1.5 mmol) was added. The reaction mixture was stirred at room temperature for 3 hours and then sodium carbonate solution (50 ml) was added. The mixture was extracted with ethyl acetate (2×60 ml) and the combined organic solutions were dried over magnesium sulphate and evaporated under reduced pressure. The residue was dried under vacuum at 60° C. to give the title compound as a yellow oil (92 mg).

LRMS (APCl) m/z 339 [M+H]⁺

Preparation 117

Isobutyl-(3-methyl-2-phenyl-butyl)amine

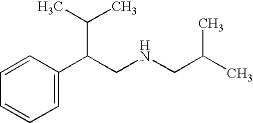

Sodium triacetoxyborohydride (600 mg, 3 mmol) followed by isobutyraldehyde (55 mg, 0.76 mmol) were added to a solution of the amine from preparation 35 (115 mg, 0.7 mmol) in dichloromethane (30 ml) and the reaction stirred at room temperature for 30 minutes. The reaction was quenched by the addition of saturated sodium carbonate solution (50 ml), and the mixture extracted with ethyl acetate (2×60 ml). The combined organic extracts were dried over magnesium sulphate and evaporated under reduced pressure to afford the title compound, 48 mg.

LRMS (APCl) m/z 220 [M+H]⁺

Preparation 118

4-Hydroxy-N-(1-pyridin-2-yl-cyclohexylmethyl)-butyramide

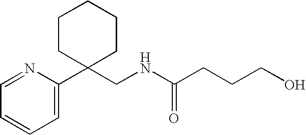

The amine from preparation 24 (2 g, 10.4 mmol) and γ-butyrolactone (0.8 ml, 10.4 mmol) were stirred at 150° C. for 4 hours. The reaction mixture was cooled to room temperature and purified by chromatography on silica gel using methanol and ammonium hydroxide in dichloromethane as eluant (gradient from 0:0.25:99.75 to 2:0.25:97.75) to give the title compound as golden oil that crystallised on standing (2.2 g).

LCMS: m/z ES⁺ 299 [M+H]⁺

Preparation 119

4-[(1-Pyridin-2-yl-cyclohexylmethyl)-amino]-butan-1-ol

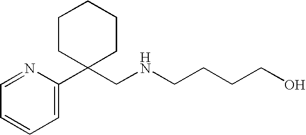

The amide from preparation 118 (1 g, 3.6 mmol) in tetrahydrofuran (15 ml) was added dropwise to a solution of lithium aluminium hydride (1M in tetrahydrofuran, 7.2 ml, 7.2 mmol) in tetrahydrofuran (10 ml) and the mixture was heated under reflux for 1 hour. The reaction mixture was cooled to room temperature and additional lithium aluminium hydride (1M in tetrahydrofuran, 3.6 ml, 3.6 mmol) was added and the reaction mixture was heated under reflux for a further 2 hours. The reaction mixture was cooled to room temperature and water (1 ml) in tetrahydrofuran (5 ml) was added dropwise. 2M Sodium hydroxide (1 ml) and then water (1 ml) were added and the mixture was stirred for 2 hours. The mixture was filtered through Hyflo Super Cel® and the filtrate was evaporated under reduced pressure. The residue was suspended in water and was extracted into ethyl acetate (3×20 ml). The combined organic solutions were dried over sodium sulphate and evaporated under reduced pressure. The residue was purified by chromatography on silica gel using methanol and ammonium hydroxide in dichloromethane as eluant (gradient from 0:0.2:99.8 to 4:0.2:95.8) to give the title compound as a colourless oil (0.44 g).

LCMS: m/z ES⁺ 263 [M+H]⁺

Preparation 120

3-(2,6-Diisopropyl-phenyl)-1-(4-hydroxy-benzyl)-1-[1-(3-trityl-3H-imidazol-4-yl)-cyclohexylmethyl]-urea

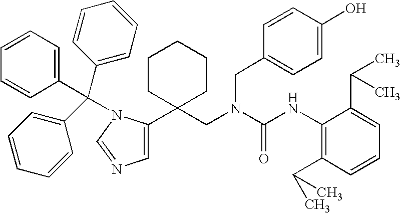

C-[1-(3-Trityl-3H-imidazol-4-yl)-cyclohexyl]-methylamine (WO 09807718, intermediate XXV) (200 mg, 0.48 mmol) was dissolved in dichloromethane (10 ml) and 4-hydroxybenzaldehyde (60 mg, 0.5 mmol) and then sodium triacetoxyborohydride (0.5 g, 2.5 mmol) were added. The reaction mixture was stirred at room temperature for 16 hours and then saturated sodium carbonate solution (50 ml) was added. The phases were separated and the aqueous solution was extracted with ethyl acetate (2×60 ml). The combined organic solutions were dried over magnesium sulphate and evaporated under reduced pressure. The material obtained was dissolved in dichloromethane (30 ml) and 2,6-diisopropylphenylisocyanate (0.15 g, 0.74 mmol) was added. The mixture was stirred at room temperature for 1 hour and then was evaporated under reduced pressure. The residue was purified by chromatography on silica gel using diethyl ether in dichloromethane as eluant (gradient from 0:100 to 10:90). The material isolated was recrystallised from dichloromethane/heptane to give the title compound as a white solid (0.16 g).

LRMS (APCl) m/z 731 [M+H]⁺

Preparation 121

2-Methoxy-3-methylbenzamide

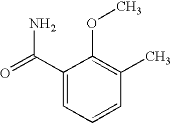

2-Methoxy-3-methylbenzoic acid (1.51 g, 9.1 mmol) was added to 1-hydroxybenzotriazole hydrate (1.22 g, 9.1 mmol) and 1-(3-dimethylaminopropyl-3-ethylcarbodiimide hydrochloride (1.4 g, 9.1 mmol) in N,N-dimethylformamide (15 ml) and dichloromethane (15 ml) and the mixture was stirred at room temperature for 20 minutes. The mixture was cooled to 0° C. and ammonia (2M solution in ethanol, 9.1 ml, 18 mmol) was added. The reaction mixture was stirred at 0° C. for 2 hours, then filtered. The filtrate was concentrated under reduced pressure and then diluted with dichloromethane. The organic solution was washed with water and brine, dried over magnesium sulphate and evaporated under reduced pressure to give the title compound as a colourless oil that crystallised on standing (1.3 g).

LCMS: m/z ES⁺ 188 [M+Na]⁺

Preparation 122

2-Methoxy-3-methyl-benzylamine

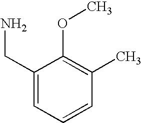

Borane-methyl sulphide complex (2M in tetrahydrofuran, 5.9 ml, 11.8 mmol) was added dropwise over 20 minutes to the amide from preparation 121 (1.3 g, 7.9 mmol) in tetrahydrofuran (20 ml) at 50° C. under a nitrogen atmosphere. The reaction mixture was heated under reflux for 5 hours and then cooled to room temperature. The reaction mixture was acidified by addition of 2M hydrochloric acid and the aqueous solution was washed with dichloromethane and then basified with 2.5M sodium hydroxide solution. The aqueous solution was extracted into dichloromethane (50 ml) and the phases were separated. The organic phase was washed with brine, dried over magnesium sulphate and evaporated under reduced pressure to give the title compound as a clear oil (357 mg).

LCMS: m/z ES⁺ 152 [M+H]⁺

Preparation 123

4-({[1-Thiophen-3-yl-cyclohexylmethyl]-amino}-methyl)phenol

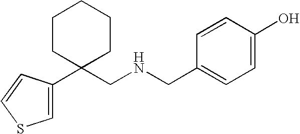

The title compound was obtained from the compound 1-thien-3-ylcarbonitrile (Eur. J. Med. Chem. 1980; 15(3) 223) and 4-hydroxybenzaldehyde following the procedure described in preparation 105.

1. A compound of formula (I): 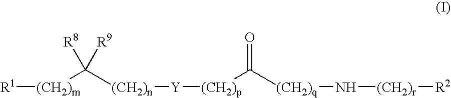
wherein
R¹ is selected from
a) aryl
b) aromatic heterocycle
c) CO₂R⁵
d) CONR⁵R⁶
e) NR⁵R⁷
f) OR⁵
g) C₁₋₆ alkyl
h) C₁₋₆ cycloalkyl and
i) —C(O)—N-morpholine
Wherein group (a) may be optionally substituted by 1-3 groups each independently selected from NR⁵R⁵, N(R⁵)C(O)R⁵, NO₂, halo, OR⁵, R⁵ and R⁴NR⁵R⁵; and group (b) may be optionally substituted by 1-3 groups each independently selected from halo, R⁵ and OR⁵;
m is 0-2;
n is 0-2;
p is 0-2;
q is 0-2;
r is 0-4
Y is NR³ or CHR³;
R² is selected from
a) C₃₋₇ cycloalkyl,
b) aromatic heterocycle, optionally fused with a phenyl group,
c) aryl, wherein said aryl group may optionally be fused with a heterocycle or a C₃₋₇ cycloalkyl group, wherein said fused cycloalkyl moiety may also incorporate a C═O group,
d) Oaryl,
e) C₁₋₆ alkyl,
f) Adamantyl, and
g) C₁₋₆ alkenyl, optionally substituted by 1 or 2 phenyl,
wherein groups (a), (b), (c), (d) and (e) may be optionally substituted by 1-3 substituents selected from: R⁵, C₁₋₆ alkenyl, aryl, OR⁴, OR⁵, OH, CF₃, halo, SO₂R⁵, NO₂, SR⁵, CN, OCF₃, CO₂R⁵, C(O)R⁵, Oaryl, OR aryl, R⁴OR⁵, C(NH)NR⁵R⁵, OC(O)C₁₋₆alkyl and NR⁵R⁷;
R³ is selected from
a) C₁₋₆ alkyl,
b) C₁₋₆ alkenyl,
c) C₁₋₆ alkynyl,
d) Aromatic heterocycle, optionally fused with phenyl,
e) Phenyl, optionally fused with phenyl, heterocycle or aromatic heterocycle,
said groups (a), (b), (c), (d) and (e) optionally substituted by 1-3 groups each independently selected from halo, CN, SR⁵, heterocycle, aromatic heterocycle, R⁵, OR⁷, C(O)NR⁵R⁷, SO₂NR⁵R⁷, NHSO₂R⁵, OH, CF₃, OR⁵, OR⁵OR⁵, NR⁵R⁷, CO₂H, CO₂R⁵, OC(O)R⁵, C₃₋₇ cycloalkyl group (wherein said cycloalkyl group may optionally be substituted by C₁₋₆ alkyl), CH₂OC(O)CH₃ and phenyl, wherein said phenyl may optionally be fused with a heterocycle, aromatic heterocycle, phenyl or C₃₋₇ cycloalkyl, said phenyl, fused phenyl, or aromatic heterocycle optionally substituted by 1-3 groups each independently selected from: phenyl, R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, HNC(O)R⁵, C₁₋₆ alkenyl, OCF₃, NO₂, halo, HNSO₂R⁵, SO₂NR⁵R⁵, C(O)NR⁵R⁵, C(NH)NR⁵R⁵, OR⁵, OC(O)R⁴-heterocycle, NR⁵R⁷, SR⁵ and tetrazole;
R⁴ is C₁₋₆ alkyl;
R⁵ is independently selected from H and C₁₋₆ alkyl, said alkyl groups optionally substituted by 1-3 groups each independently selected from halo or OH;
R⁶ is independently selected from H, heterocycle, OC₁₋₆ alkyl, C₁₋₆ alkyl, said alkyl groups optionally substituted by 1-3 groups each independently selected from halo or OH;
or R⁵ and R⁶ can be taken together with the N atom to which they are attached to form a 5, 6 or 7-membered ring optionally containing a further hetero moiety selected from O, NH, or S;
R⁷ is selected from H and C₁₋₆ alkyl said alkyl groups optionally substituted by an aryl group; and
R⁸ and R⁹ are both independently selected from H or C₁₋₆ alkyl; or R⁸ and R⁹ may combine to form a 3-7 membered cycloalkyl group. Optionally said cycloalkyl group may incorporate an atom or group selected from NR⁴, NH, O or S;
With the proviso that when R is an aryl or aromatic heterocycle group, R² is a phenyl, pyridyl or pyrimidinyl group, said groups optionally substituted, R⁸ and R⁹ combine to form a 3-7 membered cycloalkyl group, Y is NR³ and m, p, q and r are 0; then R³ cannot be C₄₋₆ alkyl or C₁ alkyl substituted by phenyl, said phenyl group optionally substituted by 1-3 groups each independently selected from halo, OC₁₋₆ alkyl and NR⁵R⁷;
and the pharmaceutically acceptable salts, prodrugs, solvates thereof.

2. A compound, salt, prodrug, or solvate according to claim 1 wherein R¹ is selected from:
a) aryl
b) aromatic heterocycle
c) CO₂R⁵,
d) OR⁵,
wherein group a) may be optionally substituted by 1-3 groups each independently selected from NR⁵R⁵, N(R⁵)C(O)R⁵, NO₂, halo, OR⁵, R⁵ and R⁴NR⁵R⁵,
and wherein group b) may be optionally substituted by 1-3 groups each independently selected from halo, R⁵ and OR⁵.

3. A compound, salt, prodrug, or solvate according to claims 1 or 2 wherein R¹ is selected from:
a) pyridyl
b) thienyl
c)phenyl
d) pyrrolyl
e) imidazolyl and
f) CO₂R⁵
wherein groups a), b), d) and e) may be optionally substituted by 1 or 2 groups each independently selected from methoxy, methyl and halo,
and wherein group c) may be optionally substituted by 1 or 2 groups each independently selected from methoxy, methyl, halo and nitro.

4. A compound, salt, prodrug, or solvate according to any one of claims 1 to 3 wherein R¹ is selected from pyridyl, methoxy-pyridyl, thienyl, phenyl, difluorophenyl, methyl-pyrrolyl, CO₂Et, methyl-imidazolyl, nitrophenyl and methoxy-phenyl.

5. A compound, salt, prodrug, or solvate according to any one of claims 1 to 4 wherein R¹ is selected from 2-pyridyl, 5-methoxy-pyridin-2-yl, 2-thienyl, 3-thienyl, 2,6-difluorophenyl, N-methyl-pyrrol-2-yl, CO₂Et, 1-methyl-imidazol-4-yl, 2-nitro-phenyl and 2-methoxy-phenyl.

6. A compound, salt, prodrug, or solvate according to claim 1 wherein m, n, p, q and r are independently selected from 0-1.

7. A compound, salt, prodrug, or solvate according to claim 6 wherein m is 0, n is 1, p is 0, q is 0 and r is 0.

8. A compound, salt, prodrug, or solvate according to claim 1 wherein Y is NR³.

9. A compound, salt, prodrug, or solvate according to claim 1 wherein R² is selected from:
a) C₃₋₇ cycloalkyl;
b) aromatic heterocycle, optionally fused with a phenyl group;
c) aryl, wherein said aryl group may optionally be fused with a heterocycle or a C₃₋₇ cycloalkyl group, wherein said fused cycloalkyl moiety may also incorporate a C═O group;
d) OPh;
e) —CH₂OHCH₂Ph;
f) adamantyl; and
g) —CH═CHPh;
wherein groups (a), (b), (c) and (d) may be optionally substituted by 1-3 substituents selected from: C₁₋₆ alkyl, C₁₋₆ alkenyl, phenyl, OR⁴, OR⁵, OH, CF₃, halo, SO₂R⁵, NO₂, SR⁵, CN, OCF₃, CO₂R⁵, C(O)R⁵, Oaryl, OR⁴aryl, R⁴OR⁵, C(NH)NR⁵R⁵, OC(O)C₁₋₆ alkyl and NR⁵R⁷.

10. A compound, salt, prodrug, or solvate according to claim 9 wherein R² is a phenyl or naphthalene group, optionally substituted by 1-3 substituents selected from C₁₋₃ alkyl, CF₃, halo, OR⁵ and NR⁵R⁷.

11. A compound, salt, prodrug, or solvate according to claim 10 wherein R² is phenyl substituted by 2 substituents selected from C₁₋₃ alkyl, halo and NR⁵R⁷.

12. A compound, salt, prodrug, or solvate according to claim 11 wherein R² is phenyl substituted by 2 substituents independently selected from Me, chloro, isopropyl and NMe₂.

13. A compound, salt, prodrug, or solvate according to claim 12 wherein R² is 2,6-diisopropyl-phenyl.

14. A compound, salt, prodrug, or solvate according to claim 1 wherein R³ is selected from:
a) C₁₋₆ alkyl;
b) C₁₋₆ alkenyl;
c) C₁₋₆alkynyl;
d) Aromatic heterocycle, optionally fused with phenyl; said aromatic heterocycle or fused heterocycle being optionally substituted by 1-3 substituents each independently selected from: halo, OC(O)CH₃ and —CH₂OC(O)CH₃; and
e) Phenyl, optionally fused with a heterocycle or aromatic heterocycle, said phenyl or fused phenyl optionally substituted by 1-3 substituents each independently selected from: C₁₋₆ alkyl, C(O)NR⁵R⁷, SO₂NR⁵R⁷ and NHSO₂R⁵;
said groups (a), (b) and (c) optionally substituted by 1-3 groups each independently selected from halo, CN, SR⁵, heterocycle, aromatic heterocycle, OR⁷, OH, CF₃, OR⁵, OR⁵OR⁵, NR⁵R⁷, CO₂H, CO₂R⁵, C₃₋₇ cycloalkyl group (wherein said cycloalkyl group may optionally be substituted by C₁₋₆ alkyl) and phenyl, wherein said phenyl may optionally be fused with a heterocycle, phenyl or C₃₋₇ cycloalkyl, said phenyl, fused phenyl, or aromatic heterocycle optionally substituted by 1-3 groups each independently selected from: phenyl, R⁴, CN, OH, OR⁴Ph, OR⁴CO₂R⁵, C₁₋₆ alkynyl, R⁴OC(O)R⁵, R⁴SR⁵, OC(O)R⁵, CF₃, OR⁷, OR⁴OR⁵, CO₂R⁵, OR⁴, CO₂R⁵, NHC(O)R⁵, C₁₋₆alkenyl, OCF₃, NO₂, halo, NHSO₂R⁵, SO₂NR⁵R⁵, C(O)NR⁵R⁵, C(NH)NR⁵R⁵, OR⁵, OC(O)R⁴-heterocycle, NR⁵R⁷, SR⁵ and tetrazole.

15. A compound, salt, prodrug, or solvate according to claim 14 wherein R³ is C₁₋₃ alkyl, optionally substituted by 1-2 groups each independently selected from OH, OR⁵, NR⁵R⁷, C₃₋₇ cycloalkyl, CO₂R⁵ and phenyl, wherein said phenyl may optionally be fused with a heterocycle, said phenyl or fused phenyl optionally substituted by 1-3 groups each independently selected from halo, NO₂, NHSO₂R⁵, SO₂NR⁵R⁵, C(O)NR⁵R⁵, C(NH)NR⁵R⁵, OR⁵ and NR⁵R⁷, or R³ is C₁₋₆ alkyl.

16. A compound, salt, prodrug, or solvate according to claim 15 wherein R³ is C alkyl, substituted by:
a) phenyl, optionally substituted by 1-3 groups each independently selected from: OH and C(O)NR⁵R⁵, or
b) C₃₋₇ cycloalkyl
or R³ is C₁₋₆ alkyl.

17. A compound, salt, prodrug, or solvate according to claim 16 wherein R³ is 4-hydroxy-benzyl, cyclopropyl-methyl, isopropyl-methyl or —CH₂Ph-(3-C(O)—NHEt).

18. A compound, salt, prodrug, or solvate according to claim 1 wherein R¹; m; n; p; q; r; Y; R²; R³; R⁴; R⁵; R⁶; R⁷; R⁸ and R⁹ are independently as defined in the Examples herein.

19. A compound, salt, prodrug, or solvate according to claim 1 wherein the compound is selected from:
Example 4
3-(2,3-Dimethylbenzyl)-1-isobutyl-1-(1-pyridin-2-yl-cyclohexylmethyl)urea
Example 7
Ethyl 1-[3-(2,6-diisopropylphenyl)-1-(4-hydroxybenzyl)-ureidomethyl]-cyclohexanecarboxylate
Example 16
3-[3-(2,6-Diisopropyl-phenyl)-1-(1-pyridin-2-yl-cyclohexylmethyl)-ureidomethyl]-N-ethyl-benzamide.
Example 39
3-(2,6-Diisopropyl-phenyl)-1-(4-hydroxy-benzyl)-1-(1-pyridin-2-yl-cyclohexylmethyl)-urea
Example 67
1-Cyclopropylmethyl-3-(2,6-diisopropylphenyl)-1-(1-pyridin-2-yl-cyclohexylmethyl)-urea
Example 162
3-(2,6-Diisopropyl-phenyl)-1-(4-hydroxy-benzyl)-1-[1-(1-methyl-1H-imidazol-4-yl)-cyclohexylmethyl]-urea
Example 163
3-(2,6-Diisopropylphenyl)-1-(4-hydroxybenzyl)-1-[1-(2-nitrophenyl)-cyclohexylmethyl)-urea
Example 168
1-[1-(2,6-Difluorophenyl)-cyclohexylmethyl]-3-(2,6-diisopropylphenyl)-1-(4-hydroxybenzyl)-urea
Example 169
3-(2,6-Diisopropylphenyl)-1-(4-hydroxybenzyl)-1-[1-(1-methyl-1H-pyrrol-3-yl)-cyclohexylmethyl)-urea
Example 170
3-(2,6-Diisopropylphenyl)-1-(4-hydroxybenzyl)-1-(1-thiophen-3-yl-cyclohexylmethyl)-urea
Example 177
3-(2,6-Diisopropylphenyl)-1-(4-hydroxybenzyl)-1-[1-(2-methoxyphenyl)-cyclohexylmethyl]urea
Example 187
3-(2,6-Diisopropylphenyl)-1-(4-hydroxybenzyl)-1-(1-thiophen-2-yl-cyclohexylmethy)urea,
Example 188
3-(2,6-Diisopropylphenyl)-1-(4-hydroxybenzyl)-1-[1-(5-methoxy-pyridin-2-yl)-cyclohexylmethy]urea
and the salts, prodrugs, or solvates thereof.

20. A compound according to any one of claims 1 to 19, without proviso, including the salts, solvates and prodrugs thereof, for use in medicine.

21. A compound according to any one of claims 1 to 19, without proviso, including the salts, solvates and prodrugs thereof, for use in the treatment of anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction.

22. The use of a compound according to any one of claims 1 to 19, without proviso, including the salts, solvates and prodrugs thereof, for the manufacture of a medicament for the treatment of anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction.

23. The use according to claim 22, for the treatment of male erectile dysfunction and female sexual dysfunction.

24. The use according to claim 23, wherein the female sexual dysfunction is female sexual arousal dysfunction.

25. A pharmaceutical composition comprising a compound of formula (I) without proviso, salts thereof, solvates thereof, and/or prodrugs thereof, according to any one of claims 1 to 19 and a pharmaceutically acceptable diluent, carrier or adjuvant.

26. A method of treating anxiety, panic attacks, social phobia, depression, psychoses, sleeping disorders, memory impairment, pulmonary hypertension, lung repair, lung development disorders, cancer treatment, prostate cancer, pancreatic cancer, hepatic porphyria, gastrointestinal secretory disturbances, gastrointestinal disorders, emesis, anorexia, pain, seasonal affective disorders (SAD), feeding disorders and sexual dysfunction, particularly male sexual dysfunction, male erectile dysfunction and female sexual dysfunction comprising administering a therapeutically-effective amount of a compound according to any one of claims 1 to 19.
